(19) United States
(12) Patent Application Publication
(10) Pub. No.: US 2026/0256523 A1
(43) Pub. Date: Sep. 3, 2026

(54) CALIBRATION AND REGISTRATION OF PRE-OPERATIVE AND INTRAOPERATIVE IMAGES

(71) Applicant: AUGMEDICS LTD., Yokneam Illit (IL)

(72) Inventors: Silvina Rybnikov, Zichron Ya'akov (IL); Gal Bar-Zohar, Yokneam Hamoshava (IL); Shay Ari, Pardes Hana Karkur (IL); Nissan Elimelech, Be'erotaim (IL); Lilach Meidan, Zichron Yaacov (IL); Nitzan Krasney, Haifa (IL); Ofer Dgani, Yokneam Illit (IL); Monica Marie Kuhnert, Chicago, IL (US); Stuart Wolf, Yokneam (IL); Tomer Gera, Kfar-Tavor (IL); Dekel Matalon, Yokneam Illit (IL)

(21) Appl. No.: 18/995,731

(22) PCT Filed: Jul. 17, 2023
(86) PCT No.: PCT/IB2023/057292

§ 371 Date: Jan. 16, 2025

Related U.S. Application Data
(60) Provisional application No. 63/389,955, filed on Jul. 18, 2022.
(60) Provisional application No. 63/389,958, filed on Jul. 18, 2022.
(60) Provisional application No. 63/428,740, filed on Nov. 30, 2022.
(60) Provisional application No. 63/438,258, filed on Jan. 11, 2023.

(51) Int. Cl. A61B 34/20; A61B 6/50; A61B 90/00; A61B 90/50; G06T 3/14; G06T 19/00; G06V 10/26; G06V 10/82; G06V 20/70

(52) U.S. Cl. CPC A61B 34/20; G06T 3/14; G06T 19/006; G06V 10/26; G06V 10/82; G06V 20/70; A61B 6/505; A61B 2034/2065; A61B 2090/365; A61B 2090/3762; A61B 2090/502; G06T 2210/41; G06V 2201/033

(57) ABSTRACT

The disclosure generally relates to systems, devices and methods to facilitate image-guided medical treatment and/or diagnostic procedures (e.g., surgery or other intervention among other considered medical usages), and to the generation of current and/or accurate anatomical images for facilitating image-guided medical treatment and/or diagnostic procedures (e.g., surgery or other intervention) and calibration and registration of imaging modalities (e.g., tomographic, volume-imaging and/or fluoroscopic modalities) used in such medical treatment and/or diagnostic procedures.

### CROSS-REFERENCE TO RELATED APPLICATIONS

This application claims priority to U.S. Provisional Application No. 63/438,258, filed Jan. 11, 2023, titled “GENERATION AND DISPLAY OF MEDICAL IMAGE DATA IN IMAGE-GUIDED SURGERY”; to U.S. Provisional Application No. 63/428,740, filed Nov. 30, 2022, titled “REGISTRATION OF TOMOGRAPHIC AND FLUOROSCOPIC IMAGES”; to U.S. Provisional Application No. 63/389,958, filed Jul. 18, 2022, titled “REGISTRATION OF TOMOGRAPHIC AND FLUOROSCOPIC IMAGES”; and to U.S. Provisional Application No. 63/389,955, filed Jul. 18, 2022, titled “FLUOROSCOPE CALIBRATION,” the disclosures of each of which are incorporated herein by reference in their entirety for all purposes.

### FIELD

The disclosure generally relates to systems, devices and methods to facilitate image-guided medical treatment and/or diagnostic procedures (e.g., surgery or other intervention among other considered medical usages), and to the generation of current and/or accurate anatomical images for facilitating image-guided medical treatment and/or diagnostic procedures (e.g., surgery or other intervention) and calibration and registration of imaging modalities (e.g., tomographic, volume-imaging and/or fluoroscopic modalities) used in such medical treatment and/or diagnostic procedures.

### BACKGROUND

Image guided surgery employs tracked surgical tools or instruments and images of the patient anatomy in order to guide the procedure. In such procedures, a proper and current imaging or visualization of regions of interest of the patient anatomy is of high importance.

Near-eye display devices and systems, such as head-mounted displays including special-purpose eyewear (e.g., glasses), are used in augmented reality systems.

See-through displays (e.g., displays including at least a portion which is see-through) are used in augmented reality systems, for example for performing image-guided and/or computer-assisted surgery. Typically, but not necessarily, such see-through displays are near-eye displays (e.g., integrated in a Head Mounted Device (HMD)). In this way, a computer-generated image may be presented to a healthcare professional who is performing the procedure, such that the image is aligned with an anatomical portion of a patient who is undergoing the procedure. Systems of this sort for image-guided surgery are described, for example, in U.S. Pat. Nos. 9,928,629, 10,835,296, 10,939,977, PCT International Publication WO 2019/211741, U.S. Patent Application Publication 2020/0163723, and PCT International Publication WO 2022/053923. The disclosures of all these patents and publications are incorporated herein by reference.

### SUMMARY

In accordance with several embodiments, systems, devices and methods are described that provide increased availability and opportunity for medical professionals to perform image-guided medical procedure navigation (e.g., medical treatment, diagnostic, and/or other intervention procedures). For example, the systems, devices and methods disclosed herein may advantageously facilitate augmented-reality assisted medical procedure navigation based on pre-operative three-dimensional (3D) imaging (e.g., pre-operative tomographic imaging, such as computed tomography (CT) scans, or magnetic resonance imaging (MRI)) of at least a portion of patient anatomy (e.g., portion of a spine or other bone, joint or soft tissue) when certain types of 3D volume imaging equipment (e.g., expensive and bulky O-arm or other CT or MR imaging equipment) is not readily available for intraoperative imaging. Some types of intraoperative 3D imaging equipment (e.g., O-arm CT machines or MRI machines) may only be available in a limited number of locations or in certain locations, such as hospital suites or operating rooms or in diagnostic centers. The systems, devices and methods disclosed herein may advantageously expand the availability of augmented-reality assisted medical or image-guided medical procedures because they involve use of more readily-available intraoperative 2D imaging equipment (such as C-arm and fluoroscopy imaging equipment) that may be used in outpatient procedure rooms, ambulatory surgical centers, or other locations. Medical professionals may, for example, navigate on a pre-operative CT scan by registering using intra-operative X-ray (e.g., fluoroscopy) and matching or calibrating the X-ray (e.g., fluoroscopy) to the pre-operative CT scan (e.g., CT/Fluoro calibration). The registration may allow the system to know which 3D voxel in a CT scan, for example, corresponds to a 2D pixel in an X-ray, or fluoroscopic, image.

The systems, devices and methods described herein may provide a similar level of accuracy and precision for image-guided or augmented reality-assisted navigation during medical procedures as those involving the more expensive and bulky intra-operative imaging equipment. The accuracy and precision may result from registration and calibration involving pre-operative images of a patient obtained prior to the procedure and intra-operative images obtained during the procedure. The calibration and registration may involve tracking markers that can be imaged by a tracking system of a wearable device (e.g., head-mounted display and tracking device, such as glasses, googles, a visor or other head-mounted or non-head-mounted device) that may be worn or donned by a surgeon or other medical professional performing the medical procedure.

The systems, devices, and methods described herein may provide for an improved real-time, image-guided display that can improve precise and accurate augmented reality-assisted navigation during a medical procedure, reduce the need for more invasive surgical procedures, have a short learning curve, save on time and resources, and mitigate risks in regard to the safety of the surgeon and other personnel in the procedure room as the system may advantageously result in less radiation exposure.

The medical procedures may include spinal surgery procedures, other orthopedic procedures (such as procedures involving the hip, knee, ankle, elbow, shoulder, foot, arm, leg), cranial procedures, dental or oral surgery procedures, ear-nose-throat (ENT) procedures, or other procedures. The systems and methods described herein may be used in connection with surgical procedures, such as spinal surgery, joint surgery (e.g., shoulder, knee, hip, ankle, other joints), orthopedic surgery, heart surgery, bariatric surgery, facial bone surgery, dental surgery, cranial surgery, or neurosurgery. The surgical procedures may be performed during open surgery or minimally-invasive surgery (e.g., surgery during which small incisions are made that are self-sealing or sealed with surgical adhesive or minor suturing or stitching). However, the systems and methods described may be used in connection with other medical procedures (including therapeutic and diagnostic procedures) and with other instruments and devices or other non-medical display environments. The methods described herein further include the performance of the medical procedures (including but not limited to performing a surgical intervention such as treating a spine, shoulder, hip, knee, ankle, other joint, jaw, cranium, etc.).

In accordance with several embodiments, the systems, devices, and methods described herein may facilitate acquiring and processing three-dimensional (3D) images (e.g., computed tomography (CT) scan, magnetic resonance imaging (MRI), ultrasound, etc.) of a patient's spine or other anatomical region prior to a surgical or other medical treatment or diagnostic procedure (e.g., hours or days or weeks or months prior), and using them in combination with two-dimensional (2D) fluoroscopic images of the patient taken during the procedure (e.g., minutes prior to actual performance of a medical intervention or during the actual performance of the medical intervention). The software in the system may include algorithms that segment the pre-operative 3D scans (e.g., CT scans or MRI scans) of an anatomical region (e.g., at least a portion of a spine) into individual anatomical components (e.g., individual vertebrae, sacrum, and ilium for the spine and pelvic region). X-ray calibration may be performed by algorithms of the software to calculate the X-ray detector parameters, distortion and X-ray source and/or detector position in a patient marker coordinate system. Registration may then be performed with the 2D intra-operative images to create a transformation for each individual anatomical component (e.g., each vertebra). Registration may involve finding a position and orientation of each individual anatomical component (e.g., each vertebra and sacrum and ilium) in the patient marker coordinate system by comparing digitally reconstructed radiographs (DRRs) with the X-rays, where the DRRs are calculated or generated from the pre-operative 3D scans using the parameters determined during X-ray calibration. Thus, the systems and methods may allow for the reconciliation of multiple coordinate systems through the programmed online X-ray calibration and registration algorithms to determine the 3D images relative to a patient marker imaged by an imaging device of an augmented reality display device to be worn by a surgeon or other medical professional, from which a 3D image volume of the anatomical region can be created or generated and a reconstructed 3D model of the anatomical region can be displayed on the augmented reality display device (e.g., wearable display device) to allow the operator (e.g., wearer) to accurately navigate one or more tools to perform a medical procedure (e.g., treatment and/or diagnostic procedure).

In accordance with several embodiments, the system can include an X-ray calibration jig that can couple to a fluoroscope (e.g., detector side of a C-arm machine). The system additionally may include a head-mounted display and/or other non-head-mounted display that allows the surgeon or other professionals to view the overlaid 3D volume images of at least a portion of the patient's anatomy (e.g., spine) relative to the patient's body, and can include a plurality of fiducial markers, which aid in the determination of the C-arm position and orientation relative to the patient and facilitates the calibration and registration processes.

Embodiments of the disclosure that are described hereinbelow provide improved methods for registration and display of images, as well as apparatus and software implementing such methods. Embodiments of the disclosure described hereinbelow additionally provide improved methods for calibration in image-guided medical treatment and/or diagnostic procedures (e.g., surgery or other intervention).

In accordance with several implementations, a method for facilitating augmented-reality assisted navigation based on pre-operative 3D imaging and intraoperative 2D imaging of at least a portion of an anatomy (e.g., spine or other bony portion) of a patient includes receiving a 3D tomographic image (e.g., CT image) of at least the portion of the anatomy (e.g., spine or other bony portion) of the patient. The portion of the anatomy may include a portion of the spine including multiple vertebrae and/or other bony structures. The method further includes segmenting the 3D tomographic image into a plurality of 3D segments, each including a respective one of the multiple vertebrae and/or other bony structures. The method also includes receiving two or more 2D fluoroscopic images of at least the portion of the anatomy (e.g., spine) of the patient. The method also includes registering each of the 3D segments with a respective vertebra or other bony structure in the two or more 2D fluoroscopic images. The method further includes generating a 3D image volume of at least the portion of the anatomy (e.g., spine) based on the registering. The method also includes presenting the 3D image volume of at least the portion of the anatomy (e.g., spine) on an augmented-reality display (e.g., see-through stereoscopic display of a wearable device, such as a head-mounted unit, glasses, visor, etc.).

The segmenting may be performed entirely automatically by one or more processors (e.g., via application of one or more trained neural networks). The processor(s) may be located on a wearable device worn by a surgeon or other clinical professional and/or on a separate workstation or portable computer. At least a portion of the segmenting may be performed manually by a user, which may be the surgeon or another clinical professional. In some implementations, the segmenting includes labeling of the multiple vertebrae and/or other bony portions (e.g., sacrum, ilium, or other bones).

In some implementations, receiving the two or more 2D fluoroscopic images includes receiving a first 2D fluoroscopic image captured from a first angle or viewpoint (e.g., anterior-posterior angle) of a C-arm fluoroscope and receiving a second 2D fluoroscopic image captured from a second angle or viewpoint (e.g., lateral or oblique lateral) of the C-arm fluoroscope different from the first angle or viewpoint.

In some implementations, presenting the 3D image volume includes overlaying an augmented reality image of the registered 3D segments on a back of the patient or other portion of the patient corresponding to the anatomical portion.

In some implementations, the method includes calibrating a frame of reference of the 2D fluoroscopic images relative to the spine of the patient prior to the registering. Overlaying the augmented reality image may include applying the calibrated frame of reference in overlaying the vertebrae or other bony portions in the registered 3D segments on the spine or other anatomical portion of the patient.

In some implementations, receiving the two or more 2D fluoroscopic images includes receiving two 2D fluoroscopic images captured from different, respective angles relative to the patient.

In some implementations, registering each of the 3D segments includes aligning the 3D segments with both of the two 2D fluoroscopic images.

In some implementations, calibrating the frame of reference of the 2D fluoroscopic images includes performing distortion correction of the two or more 2D fluoroscopic images.

In some implementations, performing distortion correction includes performing one or more spline interpolation techniques.

In some implementations, registering each of the 3D segments comprises adjusting a respective location and orientation of each 3D segment to match the respective vertebra in the two or more 2D fluoroscopic images.

In some implementations, adjusting the respective location and orientation includes processing each 3D segment to generate digitally reconstructed radiographs (DRRs) and finding an optimal match between the DRRs and the respective vertebra or other bony portions in the one or more 2D fluoroscopic images.

In some implementations, registering each of the 3D segments includes performing initial guess estimation.

In some implementations, the 3D image volume includes a reconstructed 3D model of the multiple vertebrae or other bony portions.

In accordance with several implementations, a method for facilitating augmented-reality assisted navigation based on pre-operative 3D imaging and intraoperative 2D imaging of at least a portion of an anatomy (e.g., spine) of a patient. The method includes receiving a pre-operative 3D image (e.g., CT image, MR image, 3D ultrasound image) of at least the portion of the anatomy (e.g., spine) of the patient (e.g., including multiple bony structures). The method also includes segmenting the 3D image into a plurality of 3D segments, each including a respective one of the multiple bony structures. The method further includes receiving two or more 2D intraoperative images of at least the portion of the anatomy (e.g., spine) of the patient. The method also includes registering each of the 3D segments with a respective vertebra or other bony portion in the two or more 2D intraoperative images. The method further includes generating a 3D image volume of the multiple bony structures based on the registering.

In accordance with several implementations, a method for image processing including receiving a 3D tomographic image of a patient including at least a portion of the spine made up of multiple vertebrae. The method further includes segmenting the 3D tomographic image into a plurality of 3D segments, each containing a respective one of the vertebrae. The method also includes capturing two or more 2D fluoroscopic images of at least the portion of the spine of the patient. The method further includes registering each of the 3D segments with a respective vertebra in the one or more 2D fluoroscopic image and presenting an image of at least the portion of the spine comprising the registered 3D segments on a display.

In some implementations, presenting the image comprises overlaying an augmented reality image of the registered 3D segments on the back of the patient. In some implementations, the method further includes calibrating a frame of reference of the 2D fluoroscopic images relative to the spine of the patient, wherein overlaying the augmented reality image comprises applying the calibrated frame of reference in overlaying the vertebrae in the registered 3D segments on the spine of the patient.

In some implementations, capturing the one or more 2D fluoroscopic images includes capturing two 2D fluoroscopic images from different, respective angles relative to the patient. Registering each of the 3D segments may include aligning the 3D segments with both of the 2D fluoroscopic images.

In some implementations, registering each of the 3D segments includes adjusting a respective location and orientation of each 3D segment to match the respective vertebra in the one or more 2D fluoroscopic images.

In some implementations, adjusting the respective location and orientation includes processing each 3D segment to generate digitally reconstructed radiographs (DRRs), and finding an optimal match between the DRRs and the respective vertebra in the one or more 2D fluoroscopic images.

In accordance with several implementations, a computer-implemented method for image processing includes receiving a 3D CT image of a patient including at least a portion of a spine, wherein the spine is made up of multiple vertebrae, a sacrum, and an ilium. The method also includes segmenting the 3D CT image into a plurality of 3D segments, each of the plurality of 3D segments containing a respective one of the vertebrae or the sacrum or the ilium using one or more neural networks. The method further includes capturing two or more 2D fluoroscopic images of the at least portion of the spine of the patient. The method also includes registering each of the 3D segments with a respective vertebra or ilium or sacrum in the two or more 2D fluoroscopic images. The method further includes generating a 3D image of at least portion of the spine including the registered 3D segments for presentation on a display.

In accordance with several implementations, a method for image processing includes receiving a 3D medical image of a patient including at least a portion of the spine made up of multiple vertebrae. The method also includes segmenting the 3D medical image into a plurality of 3D segments, each 3D segment containing a respective one of the multiple vertebrae. The method further includes capturing a plurality of 2D medical images of the at least portion of the spine of the patient including multiple vertebrae. The method also includes registering each of the plurality of 3D segments with a respective vertebra in the plurality of 2D medical images. The method further includes generating a 3D image of the spine comprising the registered 3D segments for output on a display.

In some implementations, the segmenting includes applying a neural network to the CT image to segment one or more areas of interest in the at least portion of the spine of the patient and applying one or more additional neural networks to the one or more areas of interest in the CT image, correspondingly, to segment at least each vertebra of the multiple vertebrae.

In some implementations, the method includes resampling the CT image to a first resolution, coarser than the CT image resolution and resampling the CT image to a second resolution, finer than the first resolution. The neural network may be applied to the CT image resampled to the first resolution and the one or more additional neural networks may be applied to the one or more areas of interest in the CT image, correspondingly, resampled to the second resolution.

In accordance with several implementations, an imaging system adapted to facilitate navigational guidance during spine surgery or other medical intervention includes or consists essentially of an X-ray calibration jig including an X-ray calibration pattern. The X-ray calibration jig is configured to be mounted, attached, coupled, or otherwise fixed to a fluoroscope (e.g., a detector portion of a C-arm fluoroscopy machine) used in an operating room of a hospital, patient care facility, ambulatory surgical center, or outpatient procedure room. The system also includes or consists essentially of a patient marker configured to be attached to a body of a patient at or adjacent a target region where spinal surgery or other medical intervention is to be performed. The system further includes or consists essentially of a registration target (e.g., registration marker), which is configured to be attached (e.g., rigidly) to the X-ray calibration jig or to the patient marker. The system also includes or consists essentially of a registration optical target (e.g., marker) having a predefined spatial relation to the registration target. The system further includes or consists essentially of at least one processor configured to execute computer-readable program instructions stored in memory, that, upon execution, cause the at least one processor to receive at least one X-ray image captured in the operating room by a fluoroscope of at least a portion of a spine of the patient, wherein the at least one X-ray image includes the X-ray calibration pattern and the registration target; receive an optical image of both the patient marker and the registration optical target; and process the X-ray image and the optical image so as to calibrate and register a frame of reference of the fluoroscope with at least the portion of the spine of the patient.

In accordance with several implementations, an imaging system includes or consists essentially of an X-ray calibration jig comprising an X-ray calibration pattern. The X-ray calibration jig is configured to be attached, mounted, coupled or otherwise fixed to a fluoroscope. The system also includes or consists generally a patient marker that is configured to be coupled, adhered or fixed to a body of a patient. The system further includes or consists essentially of a registration target, which is configured to be attached to the X-ray calibration jig or to the patient marker. The system also includes or consists essentially of a registration optical target having a pre-defined spatial relation to the registration target. The system further includes or consists essentially of a processor, which upon execution of program instructions stored on a computer-readable medium: receives a plurality of intraoperative medical images, wherein the plurality of intraoperative medical images includes i) an X-ray image captured by the fluoroscope that includes the X-ray calibration pattern and the registration target, and ii) an optical image of both the patient marker and the registration optical target; and calibrates and registers a frame of reference of the fluoroscope with the body of the patient based, at least in part, on the X-ray image and the optical image.

In accordance with several implementations, an imaging apparatus includes or consists essentially of an X-ray calibration jig comprising an X-ray calibration pattern that is configured to be attached, mounted, coupled or fixed to a fluoroscope (e.g., detector portion of a C-arm fluoroscope). The system includes or consists essentially of a registration target (e.g., registration marker) configured to be attached or coupled (e.g., rigidly attached) to the X-ray calibration jig or to the patient. The system also includes or consists essentially of at least one processor, which, upon execution of stored program instructions, is configured to receive one or more images captured in the procedural room, comprising at least one X-ray image captured by the fluoroscope, including the X-ray calibration pattern and the registration target, and a spatial relation of the registration target to a patient marker, wherein the patient marker is configured to be fixed to a body of a patient undergoing surgery or other medical intervention in a procedural room (e.g., an operating room). The at least one processor is configured to process the X-ray image and the optical image so as to calibrate and register a frame of reference of the fluoroscope with the body of the patient.

In some implementations, the apparatus further includes an Augmented-Reality (AR) display. The at least one processor is configured to apply the calibrated and registered frame of reference in presenting an image of anatomical structures in the body of the patient on the AR display.

In some implementations, the processor is configured to compute a first transformation between the frame of reference of the fluoroscope and the registration target and to compute a second transformation between the registration target and the body of the patient, and to combine the first and second transformations in order to register the frame of reference of the fluoroscope with the body of the patient.

In some implementations, the plurality of the images includes first and second X-ray images captured by the fluoroscope at different, first and second angles relative to the body. In some implementations, the at least one processor is configured to process both the first and second X-ray images so as to calibrate and register the frame of reference of the fluoroscope with the body of the patient.

In some implementations, the registration target is configured to be fixed to a bone of the patient in a pre-defined spatial relation to the patient marker.

In some implementations, the system includes a registration optical target having a pre-defined spatial relation to the registration target, wherein the one or more images captured in the procedural room (e.g., operating room) include an optical image of both the registration optical target and a patient marker wherein the patient marker is configured to be fixed to a body of a patient undergoing surgery in the operating room.

In some implementations, the method includes a registration marker. The registration marker includes the registration target and the registration optical target. The registration marker may be configured to be fixed to a surface of the body of the patient.

In some implementations, the X-ray calibration jig includes the registration target and the registration optical target is fixed to the X-ray calibration jig. In some implementations, the registration target is configured to be fixed in its location during acquisition of the X-ray image and then removed during the surgery.

In some implementations, the registration target (e.g., registration marker) includes a radiopaque pattern.

In some implementations, the radiopaque pattern includes radiopaque elements disposed in multiple different planes.

In some implementations, the fluoroscope includes an X-ray source and an X-ray detector. In some implementations, the X-ray calibration jig includes at least one ring, which contains the X-ray calibration pattern and is configured to be fitted across the X-ray detector.

In some implementations, the at least one ring comprises or consists essentially of first and second rings, which are mutually parallel and are spaced apart along an optical axis of the fluoroscope and which contain respective sub-patterns of radiopaque elements. In some implementations, the processor is configured to compare the sub-patterns in the X-ray image in order to calibrate the frame of reference of the fluoroscope.

In some implementations, the X-ray calibration jig includes multiple pads, which are disposed around a circumference of the at least one ring and are configured to lock against a peripheral surface of the X-ray detector.

In some implementations, the pads are configured to shift in a radial direction so as to engage and lock against the peripheral surface of the X-ray detector.

In some implementations, the X-ray calibration jig includes a self-centering mechanism that is configured to shift the pads together so as to center the at least one ring relative to the peripheral surface of the X-ray detector.

In some implementations, the X-ray calibration jig includes a safety strap that is configured to secure the at least one ring to the X-ray detector.

In some implementations, the X-ray calibration jig includes a flexible band that is configured to clamp around a peripheral surface of the X-ray detector.

In accordance with several implementations, a method for image-guided surgery or other medical intervention includes receiving a 3D MR image of a body of a patient including a target region that includes one or more bones on which surgery is to be performed. The method further includes processing the MR image to produce a segmented 3D image comprising bone segments and soft tissue in proximity to the bone segments. The method also includes registering the segmented 3D image with the body of the patient by aligning the bone segments in the segmented 3D image with the one or more bones in the target region of the body. The method further includes presenting the registered segmented 3D image on a display.

In some implementations, presenting the registered segmented 3D image includes overlaying an augmented reality image containing the bone segments and soft tissue on the target region of the body.

In some implementations, the one or more bones include vertebrae.

In some implementations, the one or more bones include hip bones, knee bones, ankle bones, cranial bones, arm bones, leg bones, or facial bones, and/or other bones.

In some implementations, processing the MR image includes segmenting the MR image so as to identify both the bone segments and the soft tissue in the MR image.

In some implementations, processing the MR image includes receiving and segmenting a CT image to identify the bone segments, segmenting the MR image to identify the soft tissue, and registering the MR image with the CT image to produce the segmented 3D image.

In some implementations, registering the segmented 3D image with the body of the patient includes capturing two or more fluoroscopic images of the target region, calibrating a frame of reference of the 2D fluoroscopic images relative to the body of the patient, and registering the bone segments in the segmented 3D image with the one or more bones in the 2D fluoroscopic images.

In some implementations, the display is an augmented reality display on a head-mounted unit or other wearable device. The head-mounted unit includes a pair of augmented reality glasses, a visor, a headset, or the like.

In accordance with several implementations, a method for image-guided surgery includes or consists essentially of receiving a 3D anatomical image of a target region of a body of a patient on which surgery is to be performed that includes one or more bones, processing the 3D anatomical image to produce a segmented 3D image comprising bone segments and soft tissue in proximity to the bone segments, and registering the segmented 3D image with the body of the patient by aligning the bone segments in the segmented 3D image with the one or more bones in the target region of the body.

In accordance with several implementations, a method for display based on registering 3D magnetic resonance images and 2D fluoroscopic images includes or consists essentially of receiving a 3D MR image of a body of a patient including a target region of a spine that includes one or more vertebrae on which surgery or other medical intervention is to be performed. The method also includes or consists essentially of processing the MR image to produce a segmented 3D image comprising vertebral bone segments and soft tissue in proximity to the vertebral bone segments. The method further includes or consists essentially of receiving two 2D fluoroscopic images of the target region. The method also includes or consists essentially of receiving an initial input associating a vertebral segment among the segmented 3D image with the same vertebral segment in the two 2D fluoroscopic images. The method further includes or consists essentially of estimating an orientation of the spine, associating all vertebrae in the 2D fluoroscopic images with corresponding vertebral bone segments in the segmented 3D image based on the initial input and the estimated orientation; and generating digital reconstructed radiograms from each segmented vertebral bone segment at multiple orientations. The method further includes or consists essentially of determining optimal orientations and locations of the digital reconstructed radiograms to match vertebrae in the 2D fluoroscopic images. The method also includes or consists essentially of reconstructing a spine model using the segmented 3D image at the determined optimal orientations and locations. The method further includes or consists essentially of generating the spine model for display.

In accordance with several implementations, a method for image-guided surgery of a patient includes receiving a first preoperative 3D image of soft tissues of a target region in a body of the patient on which surgery or other medical intervention is to be performed, the first image including one or more bones. The method further includes receiving a second 3D image of the one or more bones of the target region registered intraoperatively with the patient's anatomy. The method also includes aligning the first 3D image with the second 3D image by aligning the one or more bones in the first and second images. The method further includes generating a third 3D image comprising the one or more bones and soft tissue in proximity to the one or more bones. The third 3D image is registered with the patient's anatomy. The method also includes displaying the third 3D image.

In some implementations, the second 3D image is a CT image.

In some implementations, the second 3D image is an intraoperative image registered with the patient's anatomy.

In some implementations, the second 3D image is a preoperative image registered with one or more intraoperative 2D fluoroscopic images of the one or more bones.

In some implementations, the second 3D image is a segmented image including a segmentation of the one or more bones.

In some implementations, the registration of the second 3D image with the one or more 2D fluoroscopic images comprises aligning the one or more segmented bones in the second 3D image with the one or more bones in the 2D fluoroscopic images.

In some implementations, the second 3D image is the first 3D image registered with one or more intraoperative 2D fluoroscopic images of the one or more bones.

In some implementations, the second 3D image is a segmented image comprising a segmentation of the one or more bones.

Also described and contemplated herein is the use of any of the apparatus, systems, or methods for the treatment of a spine through a surgical intervention.

Also described and contemplated herein is the use of any of the apparatus, systems, or methods for the treatment of an orthopedic joint through a surgical intervention, including, optionally, a shoulder, a knee, an ankle, a hip, or other joint.

Also described and contemplated herein is the use of any of the apparatus, systems, or methods for the treatment of a cranium through a surgical intervention.

Also described and contemplated herein is the use of any of the apparatus, systems, or methods for the treatment of a jaw through a surgical intervention.

Also described and contemplated herein is the use of any of the apparatus, systems, or methods for diagnosis of a spinal abnormality or degeneration or deformity.

Also described and contemplated herein is the use of any of the apparatus, systems, or methods for diagnosis of a spinal injury.

Also described and contemplated herein is the use of any of the apparatus, systems, or methods for diagnosis of joint damage.

Also described and contemplated herein is the use of any of the apparatus, systems, or methods for diagnosis of an orthopedic injury.

In accordance with several embodiments, any of the methods described herein may include diagnosing and/or treating a medical condition, the medical condition comprising one or more of the following: back pain, spinal deformity, spinal stenosis, disc herniation, joint inflammation, joint damage, ligament or tendon ruptures or tears.

In accordance with several embodiments, a method of presenting one or more images on a wearable display is described and/or illustrated herein during medical procedures, such as orthopedic procedures, spinal surgical procedures, joint repair procedures, joint replacement procedures, facial bone repair or reconstruction procedures, ENT procedures, cranial procedures or neurosurgical procedures.

For purposes of summarizing the disclosure, certain aspects, advantages, and novel features of embodiments of the disclosure have been described herein. It is to be understood that not necessarily all such advantages may be achieved in accordance with any particular embodiment of the disclosure disclosed herein. Thus, the embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein. The methods summarized above and set forth in further detail below describe certain actions taken by a practitioner; however, it should be understood that they can also include the instruction of those actions by another party. Thus, actions such as “capturing one or more 2D fluoroscopic images” include “instructing the capturing of one or more 2D fluoroscopic images.”

The disclosure will be more fully understood from the following detailed description of the embodiments thereof, taken together with the drawings.

### BRIEF DESCRIPTION OF THE DRAWINGS

FIG. 1 is a schematic pictorial illustration of a system for image-guided surgery, in accordance with an embodiment of the disclosure.

FIG. 2A is a schematic pictorial illustration of a head-mounted unit for use in the system of FIG. 1.

FIG. 2B is a schematic pictorial illustration of another head-mounted unit for use in the system of FIG. 1.

FIG. 3A is a flowchart of an augmented-reality assisted navigation workflow involving intraoperative imaging only.

FIG. 3B is a flowchart of an augmented-reality assisted navigation workflow that involves both pre-operative and intraoperative imaging.

FIGS. 4A-4B are perspective views of an X-ray calibration jig, with FIG. 4B including a registration marker attached and FIG. 4A without the registration marker being attached.

FIG. 5 illustrates examples of bead plates of the X-ray calibration jig of FIGS. 4A and 4B.

FIG. 6 is a perspective view of another example configuration of an X-ray calibration jig with a registration marker attached.

FIG. 7 illustrates another example configuration of an X-ray calibration jig.

FIG. 8 illustrates an example configuration of the registration marker shown in FIG. 4B.

FIG. 9 illustrates an example configuration of a lower component of the registration marker shown in FIG. 6.

FIGS. 10A-10B are schematic rear views of a sliding mechanism of a mounting assembly of an X-ray calibration jig.

FIG. 11 is a schematic pictorial illustration of an X-ray calibration jig mounted to a fluoroscope.

FIGS. 12A-12C are schematic pictorial illustrations of an X-ray calibration jig with a mechanism for securing the jig to a detector portion of an X-ray machine or fluoroscope.

FIGS. 13-20 are schematic pictorial illustrations of example attachment mechanisms for attaching an X-ray calibration jig to an X-ray machine or fluoroscope.

FIGS. 21A-21B are schematic pictorial illustrations of a registration target attached by a pin to the back of a patient.

FIG. 22 is a schematic sectional illustration of a registration target attached to a pin.

FIG. 23 is a schematic sectional illustration of a registration target attached to a clamp configured for attachment to a spine of a patient.

FIG. 24 is a schematic pictorial illustration of a registration target.

FIG. 25 is a schematic pictorial illustration of a multimodal registration target attached to the back of a patient.

FIG. 26 is a schematic pictorial illustration of a multimodal registration target attached to the back of a patient.

FIG. 27 is a schematic pictorial illustration of another example configuration of a multimodal registration target.

FIG. 28 is a flow chart that schematically illustrates a method for display based on registering three-dimensional (3D) and two-dimensional (2D) images.

FIG. 29 is a schematic pictorial illustration of a CT-Fluoro calibration process.

FIG. 30 is a flow chart that schematically illustrates an example calibration method.

FIGS. 31A-31B are a flow chart and corresponding schematic pictorial illustrations of the flow chart steps illustrating bead detection.

FIGS. 32A-32H are a flow chart and corresponding schematic pictorial illustrations of the flow chart steps illustrating grid association.

FIGS. 33A-33F are a flow chart and corresponding schematic pictorial illustrations of the flow chart steps illustrating marker association.

FIG. 34 is a flow chart that schematically illustrates a method for reconstructing and displaying an augmented reality image of the spine.

FIG. 35 is a schematic representation of a segmented 3D image of a vertebra.

FIGS. 36A-36C are screen shots of an example implementation of a Graphical User Interface (GUI) display for segmenting a CT image of a spine.

FIG. 37 is a schematic pictorial illustration of a method for registering 2D and 3D anatomical images.

FIGS. 38A-38C are screen shots of an example implementation of a GUI display for registering fluoroscopic images with the segmented CT image of FIGS. 36A-36C.

FIGS. 39A-39B are screen shots of an example implementation of a GUI display showing different views of a registered vertebra (L3) in the segmented CT image overlaid on the fluoroscopic image (or vice versa) of FIGS. 38A-38C.

FIGS. 40A-40B are screen shots of an example GUI display showing different views of a registered vertebra (L4) in the segmented CT image overlaid on the fluoroscopic image (or vice versa).

FIG. 41 is a flow chart that schematically illustrates an example method for generation and display of a three-dimensional (3D) model based on registering 3D images (e.g., MR images) and two-dimensional (2D) images (e.g., fluoroscopic images).

FIG. 42 is a schematic representation of a segmented 3D image for display in image-guided surgery.

FIGS. 43A-43C are flow charts that schematically illustrate modalities for image registration, fusion, and display.

FIGS. 44A-44C are schematic representations of images of bead plates illustrating the distortion that occurs in X-ray images.

FIG. 45 is a flow chart that schematically illustrates an example method for refining image data as part of a distortion correction process.

FIG. 46 is a flow chart that schematically illustrates an example method for interpolating data as part of a distortion correction process.

### DETAILED DESCRIPTION

Embodiments of the disclosure that are described hereinbelow provide apparatus, methods and software for image calibration, registration, and display, particularly for facilitating image-guided, augmented reality-assisted navigation during medical treatment and/or diagnostic procedures (e.g., open surgery or minimally invasive surgery, such as laparoscopic surgery or endoscopic surgery).

In some systems for image-guided surgery or other medical intervention, anatomical images of structures inside the patient's body are overlaid on the surgeon's actual view of the patient's body, generating an augmented reality view that can be used to facilitate navigation by a viewer of the augmented reality view (e.g., a wearer of a head-mounted AR display device). Display of 3D anatomical images in this manner, such as computed tomographic (CT) or magnetic resonance (MR) images, can be especially useful in enabling the surgeon to visualize structures that are hidden from actual view by overlying layers of tissue or bone. During orthopedic surgery, for example, the augmented reality (AR) display may show 3D images of bone segments overlaid on the locations of the corresponding bones in a target region of the patient's body. For example, during spinal surgery, 3D images of the vertebrae may be overlaid on the skin of the patient's back for minimally invasive surgery or overlaid on the actual vertebrae for open surgery.

In image-guided surgery or other medical intervention, it is important that the anatomical images displayed to the surgeon to provide guidance and/or facilitate navigation (e.g., of medical tools and instruments) within the patient body, correspond to the current anatomy of the patient (e.g., pose and/or structure). In addition, for this sort of AR to be clinically useful, it can be important that the overlaid 3D images be properly registered with the actual anatomical structures in the body. When the 3D images are acquired during the operation, for example using an intraoperative medical imaging scanner, such as a CT or MRI scanner, proper registration will be maintained as long as the patient is stationary. In most surgeries or other medical interventions, however, the 3D images are acquired before the surgery or other medical intervention, in a different room, and, for example, the patient's pose on the operating table is often different from that in the pre-operative image (e.g., tomographic image, ultrasound image, or MR image). Three-dimensional images acquired preoperatively typically will not have a reference (e.g., a fiducial marker) which will allow the registration of the preoperative 3D images with the patient anatomy at the time of the operation. In addition, there may be a change in the relative location of individual anatomical components (e.g., vertebrae in the patient's spine) between the time the 3D images were acquired and the time of the operation or other medical intervention. Such a change may be due to a change in the patient's pose, an insertion of an implant, or any other reason. In the case of spinal surgery, the surgeon typically uses a fluoroscope in the operating room to acquire 2D images during surgery and uses these 2D images for guidance during the surgery, while viewing pre-acquired medical images (e.g., tomographic images) of the spine offline.

Embodiments of the disclosure that are described herein provide methods, systems and computer software products that can be used to register a pre-acquired 3D medical image (e.g., tomographic image or MR image) with intraoperative 2D fluoroscopic or X-ray images. In the disclosed methods, systems and computer software products, the 3D image is segmented into multiple 3D segments, for example, each containing a respective one of the vertebrae for spinal implementations. For spinal implementations, each of these 3D segments is registered with a respective vertebra in the fluoroscopic images. Specifically, the respective location and orientation of each 3D segment may be adjusted to match the respective vertebra in the fluoroscopic images and thus to account for changes in the relative location of vertebrae (e.g., due to a change in the patient's pose on the operating table relative to the pose in the 3D image). In the disclosed embodiments, two fluoroscopic images, captured from different angles or viewpoints, are used together in this registration process; but alternatively, a larger number of fluoroscopic images (e.g., three, four or more than four images) or viewpoints may be used. Similar techniques may be used for other types of surgery or medical interventions. For example, in some implementations, the 3D image may be segmented into other bony portions or components.

In the context of spine surgery, when the registration process is complete, an image of the spine comprising the registered 3D segments is presented on a display, for example by overlaying an AR image of the registered 3D segments on the back of the patient to generate an AR view. In some embodiments, to ensure proper registration between the AR image and the patient's body, the frame of reference of the 2D fluoroscopic images is calibrated relative to the patient's body (e.g., a portion of a back of the patient corresponding to a target treatment area of the spine), for example using calibration markers as described hereinbelow. This calibrated frame of reference may then be applied to a generated 3D image volume or model of the spine so that the vertebrae in the registered 3D segments are aligned properly with the spine of the patient. Similar techniques may be employed for non-spinal implementations and the calibration and registration and display may be tailored to the specific anatomy relevant to a particular medical intervention (e.g., other orthopedic surgery or intervention, cranial surgery or other intervention, ENT surgery or other intervention, oral surgery or other intervention).

In accordance with several embodiments, it may be desired to combine image information from multiple different imaging modalities and present the combined image information to the surgeon or other clinical professional, for example on an augmented-reality display of a wearable device, such as a head-mounted unit or eyewear (e.g., goggles, visor, or glasses) and/or on a non-wearable device, such as a tablet, portal monitor, or workstation display. Each imaging modality and device may have its own frame of reference, which is separate and independent from the other modalities and devices, and is typically subject to distortions of different types. These imaging modalities may include, for example, optical cameras that are used to capture visible and/or infrared images of the patient's body; a fluoroscope, which captures 2D X-ray images of the patient's body in the operating room or diagnostic room; and medical imaging scanners (e.g., tomographic scanners, such as CT scanners and MRI scanners, which may be used to capture preoperative or intraoperative 3D scans of the body. Ultrasound scanners or other 3D or 2D imaging modalities may also be used. In some aspects, the imaging modalities must be capable of imaging bone tissue.

In accordance with several embodiments, to combine image information from such different sources in a way that can give useful guidance to the surgeon or other clinical professional, it is desirable that all the imaging frames of references be calibrated and registered with the frame of reference of the patient's body (and thus all the imaging frames are registered with one another). For certain embodiments of AR displays, in which image information from these sources is visually overlaid on the body itself, precise calibration and registration can be critical to facilitate accurate and precise navigation by the surgeon or other clinical professional relying on the AR display.

Embodiments of the disclosure that are described herein provide apparatus and methods that address various benefits or advantages, particularly for operating rooms in which fluoroscopic X-ray and optical imaging are used together in image-guided surgery. For these purposes, an X-ray calibration jig (e.g., a ring adapter), comprising an X-ray calibration pattern, may be fixed (e.g., attached, mounted, or otherwise coupled) to a fluoroscope (e.g., a detector portion of a C arm fluoroscope) that is used in the operating room. A patient marker may be fixed to the body of the patient who is undergoing surgery, and a registration target (e.g., registration marker) may be rigidly attached either to the X-ray calibration jig or to the patient or to another location, such as the operating table. The registration target may be used to register the X-ray frame of reference of the fluoroscope with an optical frame of reference. One or more registration targets may be utilized, typically, one or two targets, each or some located at a different location (e.g., rigidly attached to the X-ray calibration jig or attached to the patient or elsewhere). The registration target may be in the form of a registration marker. Each registration target or marker may comprise an optical pattern and/or a radiopaque pattern, all depending on system configuration, as described below.

A processor (e.g., one or more processing devices or units) may be configured to receive images captured in the operating room, including one, two, or more X-ray images captured by the fluoroscope (which contain the X-ray calibration pattern) and an optical image of the patient marker. In some embodiments, at least one of the images (e.g., either an X-ray image or an optical image or both) contains the registration target (e.g., one or more registration markers). In a disclosed embodiment, the processor receives and uses two or more X-ray images captured by the fluoroscope at different angles or viewpoints (e.g., anterior-posterior and lateral) relative to the body. In some embodiments, the processor processes the X-ray image or images together with the optical image so as to calibrate and register the frame of reference of the fluoroscope with the body of the patient. For this purpose, the processor typically computes a first transformation between the frame of reference of the fluoroscope and the registration target (e.g., marker) and a second transformation between the registration target (e.g., marker) and the body of the patient, and then combines these two transformations in order to register the frame of reference of the fluoroscope with the body of the patient.

In some embodiments, the processor applies the calibrated and registered frame of reference of the fluoroscope in presenting an image of anatomical structures (e.g., individualized vertebrae, a portion of a spine (lumbar, sacral, lumbosacral, cervical, thoracic), a whole spine, pelvic bones, leg bones, arm bones, hip bones, knee joints, ankle or foot bones, hand bones, brain tissue, cranial bones, oral and maxillofacial bones, bone joints such as sacroiliac joints, organs or other soft tissue, etc.) in the body of the patient on a display, such as an AR display. Additionally or alternatively, other sorts of information may be integrated into the AR image. For example, the display may incorporate information from a pre-acquired 3D tomographic image, such as a CT image or an MRI image, or other medical image. For this purpose, it is desirable that the tomographic or other medical image also be registered with the body of the patient. This sort of registration may be accomplished, for example, by registering the pre-acquired 3D tomographic or other medical image with intraoperative 2D fluoroscopic images, as described below.

The terms “image” and “images,” as will be used hereinafter, may include two-dimensional images and/or three-dimensional images, including computer-generated two-dimensional or three-dimensional renderings or models.

Several embodiments are particularly advantageous because they include one, several or all of the following benefits: (i) improved visualization of structures within the body during surgery; (ii) enhanced accuracy in planning and carrying out surgical procedures; (iii) increased availability of AR-assisted navigation without sacrificing accuracy or precision; (iv) enrichment of the surgeon's understanding of 3D features of the patient's anatomy; (v) reduced patient radiation compared to 3D-CT intraoperative imaging; and/or (vi) reduced overall procedure time due to typical availability of C-arm machines compared to O-arm or other CT machines.

### Fluoroscope Calibration

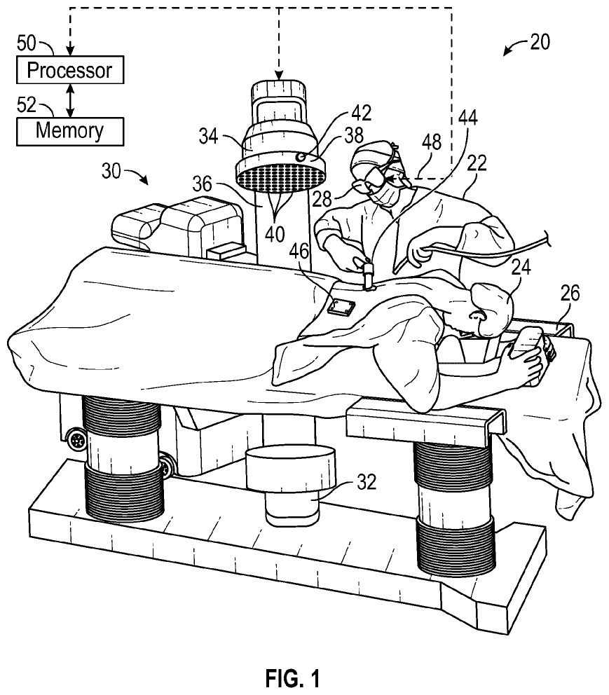

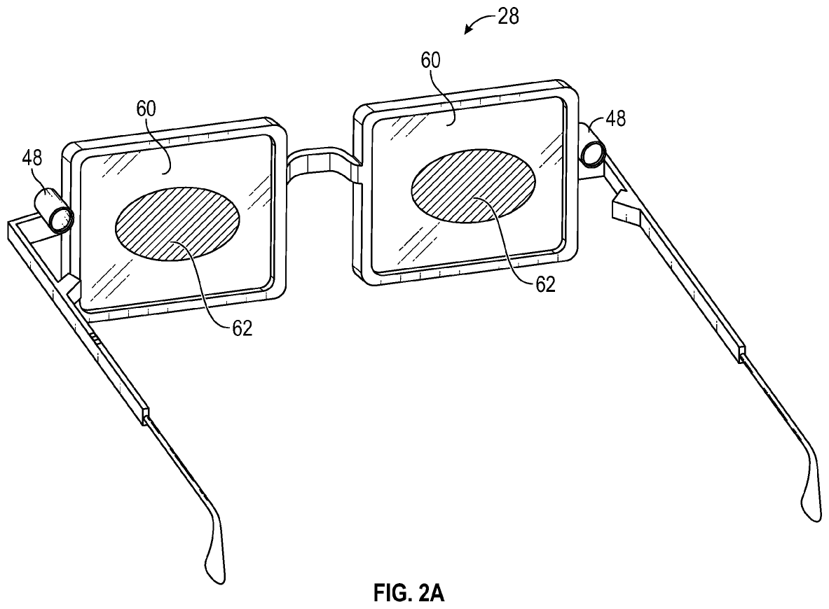

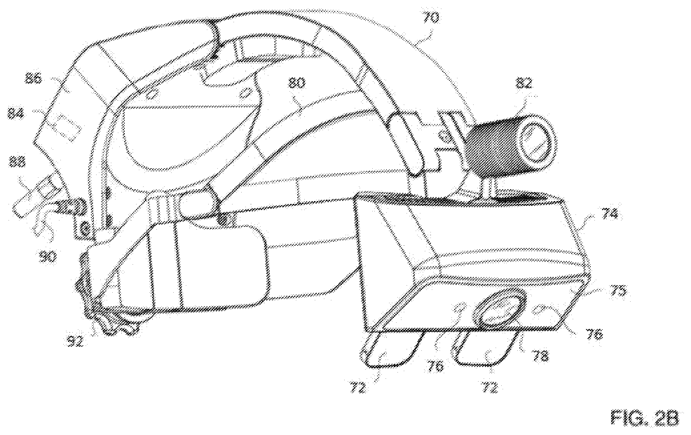

FIG. 1 is a schematic pictorial illustration of an AR system 20 for image-guided surgery or other medical intervention using AR-assisted navigation, in accordance with an embodiment of the disclosure. In the pictured scenario, a surgeon or other clinical professional 22 is preparing to operate on the spine of a patient 24, who is lying on an operating table 26. The surgeon 22 views the patient's back through a head-mounted AR display unit 28, examples of which are shown in greater detail in FIG. 2A or 2B. Before and/or possibly during the surgery, a fluoroscope 30 is used to acquire 2D images of at least a portion of the spine of patient 24 (as well as potentially other bones, vessels, and/or soft tissue or other internal body structures) from two or more different angles (e.g., anterior-posterior view and lateral view). Fluoroscope 30 comprises an X-ray source 32 and an X-ray detector 34, which are held on opposing sides of the patient's body by a C-arm 36.

For purposes of providing image guidance during surgery or other medical intervention, and specifically for the purpose of AR image display and assisted navigation, it can be important that the frame of reference of the images captured by fluoroscope 30 be calibrated and registered relative to the physical frame of reference of the body of patient 24. In accordance with several embodiments, there are two aspects to this calibration: (1) correction of distortion in images captured by fluoroscope 30 itself and computing the intrinsic parameters of the fluoroscope 30 (e.g., focal length, principal point, and skew), and (2) registration of fluoroscope 30 with the patient's body. For these purposes, an X-ray calibration jig 38 (e.g., fluoroscopic ring adapter) may be fitted over, or otherwise mounted or attached to the X-ray detector 34. In the pictured embodiment, jig 38 comprises or consists essentially of an X-ray calibration pattern in the form of an array of X-ray opaque beads or other fiducial elements 40 in a predefined layout, as described further hereinbelow. The bead pattern appears in the fluoroscopic images captured by the X-ray detector 34. A processor 50, which may include one or more than one processing devices or units, receives and processes these images in order to correct X-ray image distortion, including the extrinsic and intrinsic parameters of the fluoroscope 30 (e.g., X-ray detector 34), and determine the location and orientation of the optical axis of fluoroscope 30.

In addition, in the embodiment shown in FIG. 1, X-ray calibration jig 38 comprises or consists essentially of a registration target in the form of an optical marker 42, which comprises an optical pattern and is fixed to jig 38 in a known position and orientation relative to the pattern of beads 40. Alternatively or additionally, the registration target 42 may comprise a radiopaque pattern and may be fixed to the body of patient 24 or fixed to a patient table or another location.

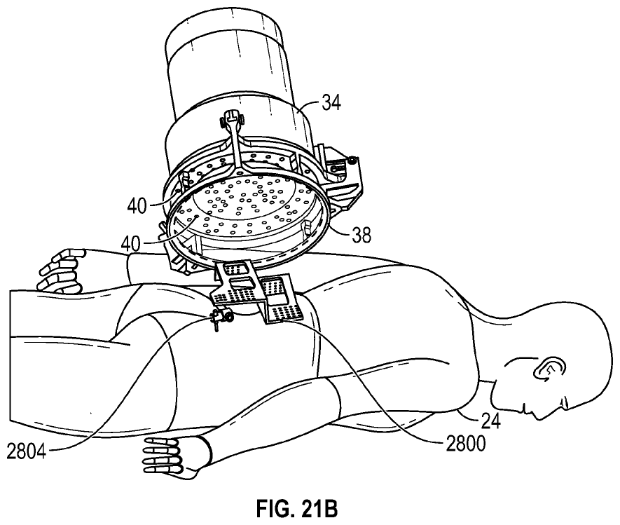

In several embodiments, processor 50 uses optical marker 42 in conjunction with an optical patient marker or other fiducial marker on the body of patient 24 in registering the optical axis of fluoroscope 30 with the body of patient 24. In some embodiments, surgeon 22 may attach a patient marker 44 to a bone in the body of patient 24, for example to the patient's spine, using a suitable clamp (e.g., spinous process clamp) or pin (e.g., iliac pin). A marker of this sort is described, for example, in U.S. Pat. No. 10,939,977, whose disclosure is incorporated herein by reference. Additionally or alternatively, surgeon 22 may fix a registration marker 46 to the patient's body surface, for example as shown in FIG. 1. A registration procedure utilizing a marker attached to the patient's back, is described, for example, in U.S. Patent Application Publication 2021/0161614, whose disclosure is likewise incorporated herein by reference. Alternatively, surgeon 22 may use a registration marker mounted on the patient's spine via a supporting or mounting structure such as a clamp or a pin, as shown in FIGS. 21A-23. A registration procedure utilizing a marker mounted on a patient's spine via such a supporting or mounting structure is disclosed, for example, in U.S. Patent Application Publication 2022/0142730, whose disclosure is likewise incorporated herein by reference. It should be noted that the use of a registration marker such as registration marker 46 may make the use of optical marker 42 unnecessary. In some implementations, a camera 48 (e.g., infrared camera or other optical camera) captures images including both optical marker 42 on calibration jig 38 and marker 44 and/or marker 46 attached to patient 24. Although camera 48 in FIGS. 1, 2A and 2B is mounted on head-mounted AR display unit 28, these images may alternatively be captured by one or more suitable optical cameras (e.g., infrared camera) mounted elsewhere on the head or body of surgeon 22 or mounted elsewhere in the operating room (e.g., in a stationary manner).

In accordance with several embodiments, processor 50 processes the images captured by X-ray detector 34 and, according to some embodiments, also by optical camera 48 (e.g., infrared camera) in order to calculate the location and orientation of fluoroscope 30 relative to the patient's body and thus to calibrate and register the fluoroscopic frame of reference relative to the frame of reference of the patient's body. Specifically, processor 50 computes a first transformation between the frame of reference of fluoroscope 30, as represented by jig 38, and the optical frame of reference of the registration target (e.g., optical marker 42), which is fixed to the jig 38 in this embodiment. In this case, the geometrical relationship between jig 38 and optical marker 42 is fixed and known in advance, thus simplifying the computation of the transformation. Alternatively, processor 50 may compute the first transformation between the X-ray calibration pattern of beads 40 on jig 38 and a radiopaque pattern on a registration marker (e.g., registration marker 46) in a calculated, determined, or predefined spatial relation to patient marker 44, as exemplified in some of the figures that follow. According to some embodiments, processor 50 computes a second transformation between the registration target (e.g., registration marker 46) and the body of the patient (e.g., using the optical pattern of registration marker 46 and the optical pattern of patient marker 44). Processor 50 may then combine these two transformations in order to register the frame of reference of fluoroscope 30 with the body of patient 24 (e.g., the portion of the patient anatomy relevant to a medical intervention to be performed, such as a spine, cranium, mouth, orthopedic joint, of the patient).

In addition to receiving 2D X-ray images from fluoroscope 30, processor 50 may also receive 3D tomographic or other medical images of patient 24 (e.g., CT or MRI images), and store these 3D images in a memory 52. For spinal interventional procedures, processor 50 may segment the 3D images and register the 3D segments with respective vertebrae in the 2D fluoroscopic images. The processor 50 may then present an image of the spine comprising the registered 3D segments on head-mounted AR display unit 28, such that the vertebrae in the 3D images are aligned with the actual vertebrae of the patient's spine. Such presentation may facilitate AR-assisted navigation during a surgical procedure or other medical intervention (e.g., therapeutic and/or diagnostic intervention). Details of this process are described herein. Similar processes may be performed for other joints, bones, or tissue.

Alternatively or additionally, processor 50 may present image information on a different sort of display, for example on an AR display that is mounted on patient 24 or on operating table 26 above the surgical site or at another location within the operating room, such as a stationary display (e.g., a workstation display) located in the operating room.

Processor 50 may comprise one or more general-purpose computer processors, which is or are programmed in software (via computer-readable program instructions) to carry out the functions of segmentation, calibration, registration, and/or display that are described herein. This software may be stored on tangible, non-transitory computer-readable media, such as optical, magnetic, or electronic memory media. Additionally or alternatively, at least some of the functions of processor 50 may be carried out using special-purpose computing hardware, such as a graphics processing unit (GPU), which may include, for example, multiple units.

FIG. 2A is a schematic pictorial illustration showing details of head-mounted AR display unit 28, in accordance with an embodiment of the disclosure. Head-mounted display unit 28 is in the form or substantially in the form of glasses, spectacles, goggles, or other eyewear. Head-mounted display unit 28 includes see-through displays 60, for example as described in the above-mentioned U.S. Pat. No. 9,928,629 or PCT International Publication WO 2022/053923. The see-through displays 60 may comprise optical see-through displays, video see-through displays, or a hybrid combination of both. The see-through displays 60 may comprise a stereoscopic display. The head-mounted display unit 28 may comprise eyewear (e.g., glasses or goggles) such as displayed in FIG. 2A. The head-mounted display unit 28 may alternatively comprise a headset configured to be mounted over the head of the surgeon 22 instead of just on the ears and nose (and/or the forehead) of the surgeon 22, such as shown in the unit of FIG. 2B. Displays 60 may be controlled by processor 50 (e.g., by a processor unit of processor 50 disposed on head-mounted AR display unit 28, not shown in the figures) to display an AR image to surgeon 22, who is wearing the head-mounted AR display unit 28. In some implementation, this AR image is projected onto an overlay area 62 of displays 60 in alignment with the anatomy of the body of patient 24, which is visible to surgeon 22 through displays 60. The AR image may include, for example, anatomical features, such as images or 3D models or representations of bones taken from tomographic or volumetric images and/or graphical representations of tools inside the patient's body, as well as surgical guidance and planning data or other information. The AR image may be overlaid on the actual locations of the anatomical features of patient 24 that are viewed by surgeon 22. In some implementations, the AR image is presented directly into or onto the retina of one or both of the patient's eyes.

To align the AR image with the patient's anatomy, one or more cameras 48 (e.g., infrared or other optical cameras) may be configured to capture respective images of a field of view (FOV), which includes marker 42 and for registration purposes, images which includes marker 46 and/or marker 44. In some embodiments, processor 50 processes the images of one or more of the markers to register the location and orientation of display unit 28 with the patient's body. Based on this registration, processor 50 is able to select the appropriate features to display in the AR image in overlay area 62 (which may be displayed directly on a wearer's retina) and to set the appropriate magnification, translation, and orientation to match the underlying structure of the patient's anatomy as seen from the point of view of surgeon 22 or other clinical professional.

FIG. 2B is a schematic pictorial illustration showing details of a head-mounted display (HMD) unit 70, according to another embodiment of the disclosure. HMD unit 70 may be worn by surgeon 22 and may be used in place of HMD unit 28 (FIG. 2A). HMD unit 70 comprises an optics housing 74 which incorporates a camera 78, and in the specific embodiment shown, an infra-red camera. Thus, housing 74 also comprises an infra-red transparent window 75, and within the housing (e.g., behind the window) are mounted one or more (e.g., two) infrared projectors 76. Mounted on housing 74 are a pair of augmented reality displays 72, which allow surgeon 22 to view entities, such as part or all of patient 24 through the displays 72, and which are also configured to present to surgeon 22 AR images or any other information.

HMD unit 70 includes a processor 84, mounted in a processor housing 86, which operates elements of the HMD unit. An antenna 88 may be used for communication with processor 50 (e.g., with a processor mounted on a workstation). The processor 84 may be a processing unit of processor 50 or may communicate with processor 50. HMD unit 28 of FIG. 2A may also include one or more processors, similar to HMD unit 70.

Optionally, a flashlight 82 may be mounted on the front of HMD unit 70. The flashlight 82 may project visible spectrum light onto objects so that surgeon 22 is able to clearly see the objects through displays 72. Elements of the HMD unit 70 are typically powered by a battery (not shown in the figure) which supplies power to the elements via a battery cable input 90. HMD unit 28 of FIG. 2A may also include a flashlight, similar to HMD unit 70.

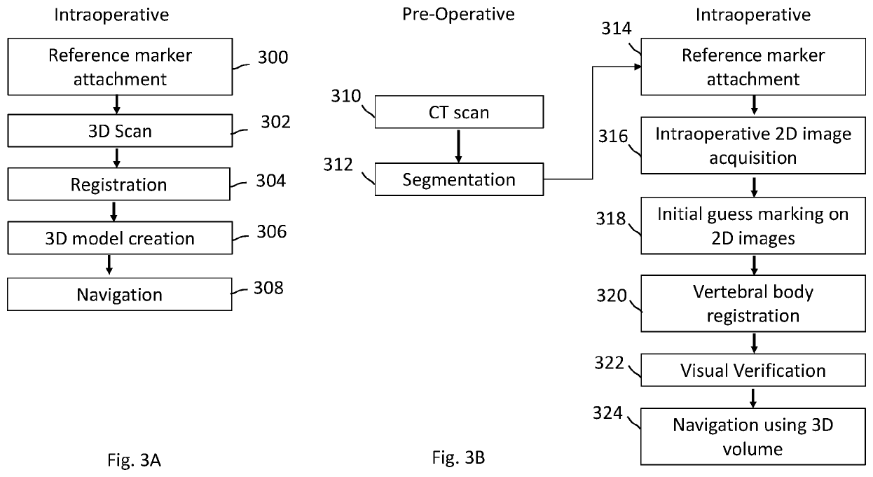

FIG. 3A is a flowchart of an augmented-reality assisted navigation workflow where each of the steps, including the acquisition of a 3D scan of a patient 24, takes place intraoperatively during an operation or other medical intervention. At intraoperative step 300, one or more reference markers are attached to the patient 24 and/or to a medical tool. At step 302, a 3D scan of the patient 24 is obtained and stored in memory (e.g., imported to memory of a head-mounted unit and/or a workstation). At step 304, registration occurs between the markers and the 3D scan, and at step 306, a 3D image model is created and displayed based on the registration to facilitate AR-assisted navigation. At step 308, the surgeon 22 navigates based on the AR display (e.g., inserts screws into the patient 24). In some instances, the intraoperative 3D scan results in a costly and time-consuming operation, taking approximately 30 to 35 minutes. This time may include transporting and positioning the intraoperative imaging apparatus (e.g., an O-arm machine), draping, imaging, breaking down the equipment, and re-scrubbing. In some instances, the intraoperative 3D scanning equipment may not be readily available at the location where the procedure is desired to be performed.

FIG. 3B is a flowchart of a CT-Fluoro navigation workflow and illustrates a process that can eliminate the need for costly and time-consuming intraoperative 3D scans (e.g., CT or MRI scans), as the process involves acquiring a pre-operative 3D scan (e.g., CT scan) and uses a more readily available and more common intraoperative imaging apparatus—a C-arm fluoroscope or other 2D X-ray machine. In this workflow, a patient 24 undergoes a pre-operative 3D imaging scan (e.g., CT scan) at step 310. At step 312, the 3D imaging scan (e.g., CT scan) is stored in memory (e.g., imported into memory of a workstation) and undergoes segmentation. The segmentation can be automatically performed by software or artificial intelligence (AI), such as by trained neural networks, and a user can make manual adjustments as needed during segmentation (e.g., using manual visualization and adjustment techniques). The process then enters the intraoperative phase. At step 314, a reference marker is attached to a calibration jig (e.g., fluoroscope ring adapter), which is coupled to the C-arm of a fluoroscope (e.g., an X-ray detector portion of the fluoroscope). At step 316, two or more intraoperative 2D images are acquired using the C-arm or other fluoroscope or 2D imaging device. A user (e.g., the surgeon 22 or other clinical professional) generates an initial guess marking on the 2D images at step 318 by selecting one of the segmented vertebra or other bony portion and marking its position on each of the fluoroscopic images. The initial guess marking could also be performed automatically by the processor 50. In some implementations, the initial guess is computed by taking the Z direction of the marker (e.g., registration marker coupled to the fluoroscope), which should correspond to the patient's chest-to-back direction and taking the Y direction of the X-ray emitter or source, which should correspond to the patient's legs-to-head direction. By combining these two directions, a coordinate system may be defined that is approximately parallel to the patient's direction in the pre-operative CT or other pre-operative image. At step 320, a processor, upon execution of stored program instructions, registers each vertebral body or other bony structure or portion. In some embodiments, a user can manually assist in this registration step. At step 322, the user (e.g., the surgeon 22 or other clinical professional) then visually verifies each vertebral body registration, and finally, the user (e.g., the surgeon 22 or other clinical professional) performs the procedure by navigation using a generated 3D volume of the vertebrae at step 324. Because the 3D scan is performed pre-operatively, the intraoperative imaging comprising the intraoperative fluoroscopic imaging can be expected to take less time (e.g., approximately 10 to 15 minutes) and does not require availability of the 3D intraoperative imaging machines, saving time and expenses.

### X-Ray Calibration Jigs

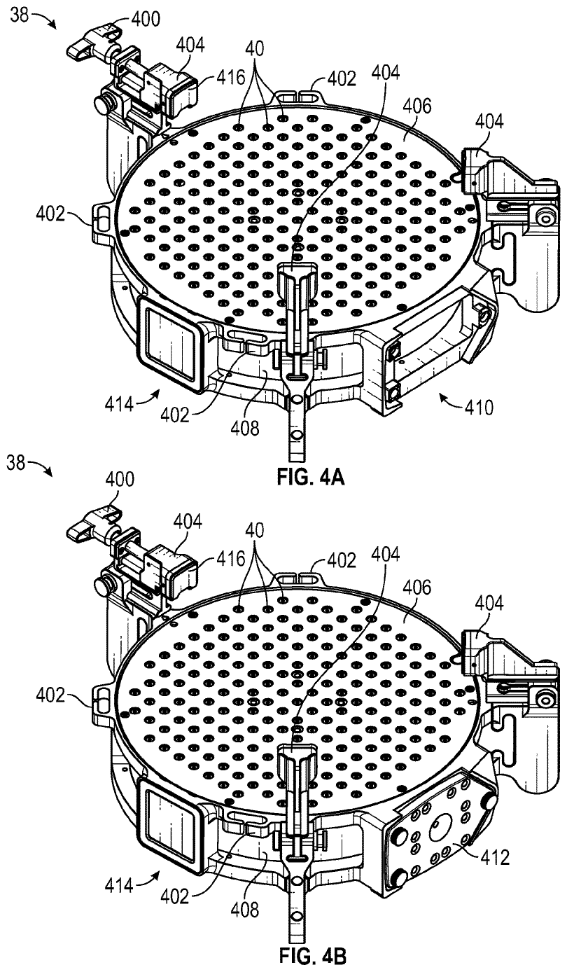

FIG. 4A is a perspective view of an embodiment of an X-ray calibration jig 38. X-ray calibration jig 38 comprises or consists essentially of two bead plates, upper bead plate 406 and lower bead plate 408. Each of the bead plates comprises or consists essentially of a pattern of radiopaque or X-ray opaque beads 40 (e.g., metal beads). Strap holders 402 may be disposed at the upper portion of the X-ray calibration jig's 38 chassis, external to the upper bead plate 406, and can accommodate straps to couple X-ray calibration jig 38 to the X-ray detector 34 on C-arm 36, providing support and/or stability. In some embodiments, the jig's chassis is manufactured as a single part to improve accuracy of the bead and marker 412 placement. The bead plates 406, 408 may be constructed and adapted to interface with the chassis of the jig 38 such that the bead plates 406, 408 are positioned according to a known or predetermined configuration.

Jig 38 further includes a ring tightening device 400 and static clamps 404, the static clamps 404 further comprising pads 416. The jig 38 may be adjustable to accommodate fluoroscopes of differing sizes (e.g., 9-inch versions or 12-inch versions). Pads 416 comprise a non-slipping material (e.g., silicone) to provide stable attachment of the jig 38 to the fluoroscope (e.g., detector portion of the C-arm or other fluoroscope). X-ray calibration jig 38 comprises or consists essentially of a marker holder 410. In some embodiments, the marker holder 410 can accommodate a marker 412 through a 3-pin (e.g., 3-screw) attachment mechanism to facilitate increased accuracy and precision, as well as facilitating ease of manufacturing reproducibility. In some configurations, marker holder 410 can accommodate markers through other various attachment mechanisms. For example, in one configuration, marker holder 410 can accommodate a marker through a 2-pin attachment mechanism, a 4-pin attachment mechanism, snap-fit mechanisms, latch mechanisms, and/or the like. In some embodiments, X-ray calibration jig 38 comprises a Quick Response (QR) code element 414 or other machine-readable element or information element. QR code element 414 can store information such as parameter information pertaining to marker location or fluoroscopic camera parameters or manufacturing parameters of the marker for a specific X-ray calibration jig 38 (for example, if manufacturing tolerances or reproducibility is not sufficiently precise or achievable). The QR code element 414 could include other information as desired and/or required. The QR code element 414 may be scanned or read by a suitable imaging device or camera of the head-mounted display unit 28, 70 or a separate imaging device or camera in communication with processor 50 and the information can be stored in memory of the head-mounted display unit 28, 70 and/or memory 52.

FIG. 4B shows the X-ray calibration jig 38 of FIG. 4A with the marker 412 attached to the marker holder 410 via the 3-pin attachment mechanism. In some implementations, the marker 412 is configured or adapted for use as an “over-the-drape” marker. In this implementation, a surgical drape (e.g., for sterile field maintenance purposes) can be placed over the jig 38 and the marker 412 is connected to the marker holder 410 through the drape, such that the pins or screws of marker 412 are pushed through the drape to couple the marker 412 to the marker holder 410.

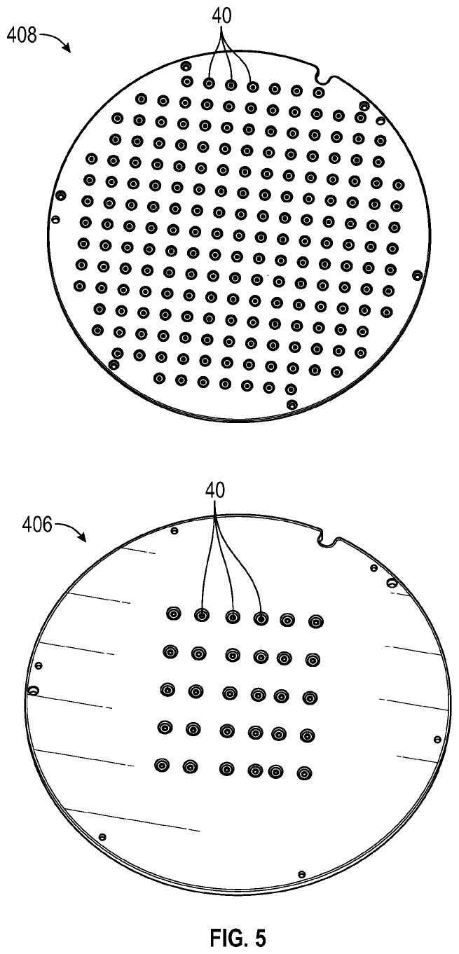

FIG. 5 illustrates examples of the lower bead plate 408 and the upper bead plate 406 of the jig 38 of FIGS. 4A and 4B. In some implementations, the upper bead plate 406 is used for distortion correction and the lower bead plate 408 is used for calculating the parameters (intrinsic and extrinsic parameters) of the camera or detector of the C-arm or other imaging device (e.g., fluoroscopic or other X-ray imaging device or other 2D imaging device). The grid patterns and sizes of beads 40 or other elements may vary as desired and/or as required, as long as they are different between the upper bead plate 406 and the lower bead plate 408. For example, the upper bead plate 406 may have more beads 40 than the lower bead plate 408 and the beads 40 of the upper bead plate 406 may be smaller than the beads 40 of the lower bead plate 408 for differentiation. In some embodiments, the grid of the upper bead plate 406 may form a perfect grid layout, with constant bead sizes and the same gap distance between vertical and horizontal lines. In some embodiments, beads 40 are formed using radiopaque materials (e.g., titanium, stainless steel, tungsten, etc.). Utilization of radiopaque beads facilitates detection of various bead patterns. In some embodiments, the plates on which the beads are disposed are formed using material that is radiolucent and durable under X-ray radiation. In some embodiments, the plate material is a plastic or polymer (e.g., polyethylene terephthalate (PET)) or glass or ceramic. The beads may be replaced with other sorts of radiopaque elements other than beads. The bead cutouts may also have shapes other than circles. Alternatively, the X-ray calibration jig 38 may comprise a single ring or three or more rings, as well as other suitable sorts of geometrical structures other than rings.

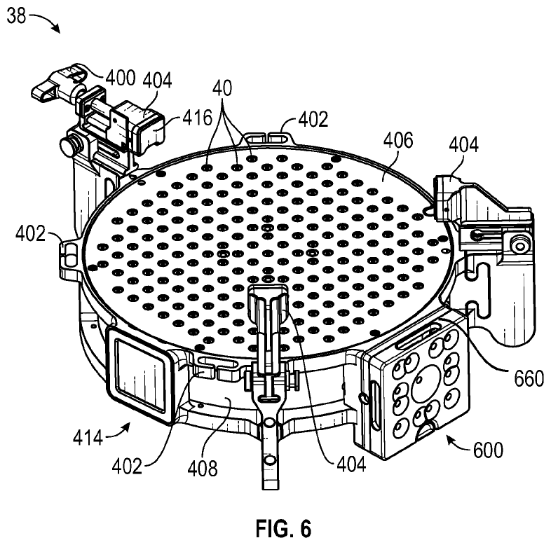

FIG. 6 illustrates another configuration of an X-ray calibration jig 38. Unless otherwise noted, the components of FIG. 6 are the same as or generally similar to the components of FIGS. 4A-4B. FIG. 6 illustrates an example of a marker 600 that is adapted and configured for use under a drape, meaning that the sterile surgical drape can be draped completely over the jig 38, including over at least a portion of the marker 600. The surgical drape may be transparent. In some implementations, a mechanism may be implemented to allow a portion of the drape to be stretched over the marker 600 such that the portion of the drape surrounding the marker 600 does not fold on itself. In some implementations, there is a groove 660 between an outer component of the marker 600 and an inner component of the marker 600. The groove 660 may facilitate use of an elastomer ring to stretch the drape tight over the marker 600 such that there are no folds or deviations that may impact image or operational quality. In addition, the attachment mechanism may be a snap-fit attachment mechanism, wherein the outer component of the marker 600 snaps onto the inner component. In some implementations, there is no outer and inner separable component but an integrated component that snaps on to an appropriate mounting structure on the calibration jig 38. In accordance with several implementations, the snap-fit attachment mechanism may allow for quick installation and removal of the marker 600 to the calibration jig 38. In some implementations, the snap-fit attachment mechanism includes a release button or latch that may provide an audible click when proper attachment is achieved and that may be actuated to cause simple detachment of the marker 600.

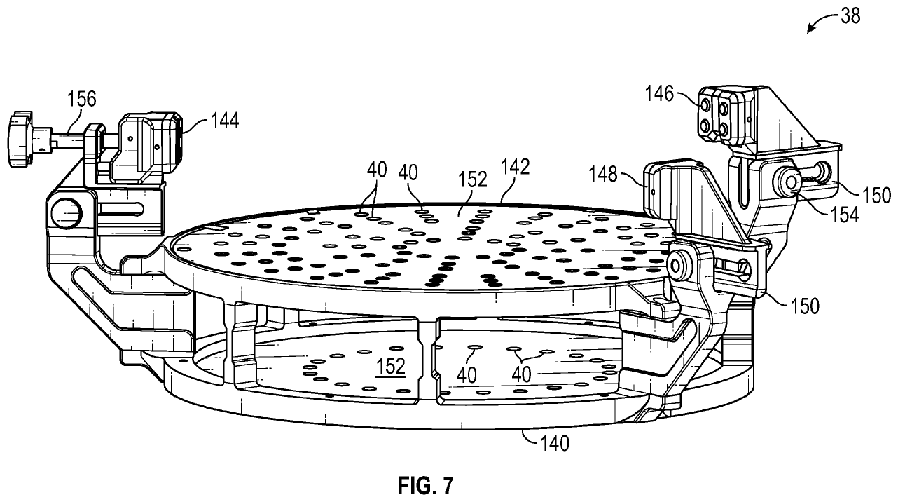

FIG. 7 is a schematic pictorial illustration showing details of X-ray calibration jig 38, in accordance with another embodiment of the disclosure. Any of the structural and operational features described in connection with FIG. 7 may also be incorporated into the calibration jigs 38 of the preceding figures. X-ray calibration jig 38 of FIG. 7 comprises or consists essentially of two rings 140, 142, which are fitted across X-ray detector 34 (as shown in FIG. 1) and contain different, respective X-ray calibration sub-patterns made up of radiopaque beads 40 or other sorts of radiopaque elements. Alternatively, the X-ray calibration jig 38 may comprise a single ring or three or more rings, as well as other suitable sorts of geometrical structures other than rings. In the pictured embodiment, beads 40 are contained in substrates 152 that are relatively transparent to X-rays, such as glass or polymer substrates. The bead patterns of the jig 38 of FIG. 7 are different than those of the jig 38 of FIGS. 4A and 4B. As shown, the lower plate has a circular pattern as opposed to a more square or rectangular grid pattern and the upper plate also has a radial pattern as opposed to a more rectangular or square grid pattern.

Rings 140 and 142 are mutually parallel and are spaced apart by a known distance along the optical axis of fluoroscope 30, so that the respective sub-patterns overlap in the X-ray images captured by detector 34. In accordance with several embodiments, the distortion of each of the sub-patterns and the relation between the projections of the two sub-patterns in the fluoroscopic X-ray images are advantageously indicative of aberrations and distortions of fluoroscope 30.

Processor 50 may be configured or programmed to compare the sub-patterns in the X-ray images to their ideal shapes and to one another in order to calibrate the frame of reference of the fluoroscope. This calibration procedure may involve both computing the location and orientation of the optical axis of fluoroscope 30 (e.g., computing extrinsic parameters of rotation and translation and intrinsic parameters of the fluoroscope 30) and computing and correcting for distortions in the fluoroscopic images of the patient's body. The distortion correction may be performed based on principles (e.g., spline interpolation methods) described, for example, in “Calibration and Gradient-Based Rigid Registration of Fluoroscopic X-ray to CT, for Intra Operative Navigation” by Harel Livyatan, available at https://www.cs.huji.ac.il/labs/casmip/wp-content/uploads/2015/08/msc-thesis-2003-harel-livyatan.pdf, which is incorporated by reference herein.

To secure X-ray calibration jig 38 to X-ray detector 34, the jig 38 may comprise multiple pads 144, 146, 148, which are disposed around the circumference of ring 142 and lock against the peripheral surface of the X-ray detector 34. Pads 144, 146, 148 in this example comprise elastomeric friction pads inserted in a polymer base to grip the X-ray detector 34 securely. The pads 144, 146, 148 are mounted on slides 150, which enable the pads 144, 146, 148 to shift in a radial direction so as to engage X-ray detectors of different diameters (e.g., 9 inches or 12 inches). Lock buttons 154 on slides 150 can be released to enable pads 144, 146, 148 to shift along slides 150 and then actuated to secure the pads in the selected position. An adjustment knob 156 advances pad 144 to lock jig 38 securely in place.

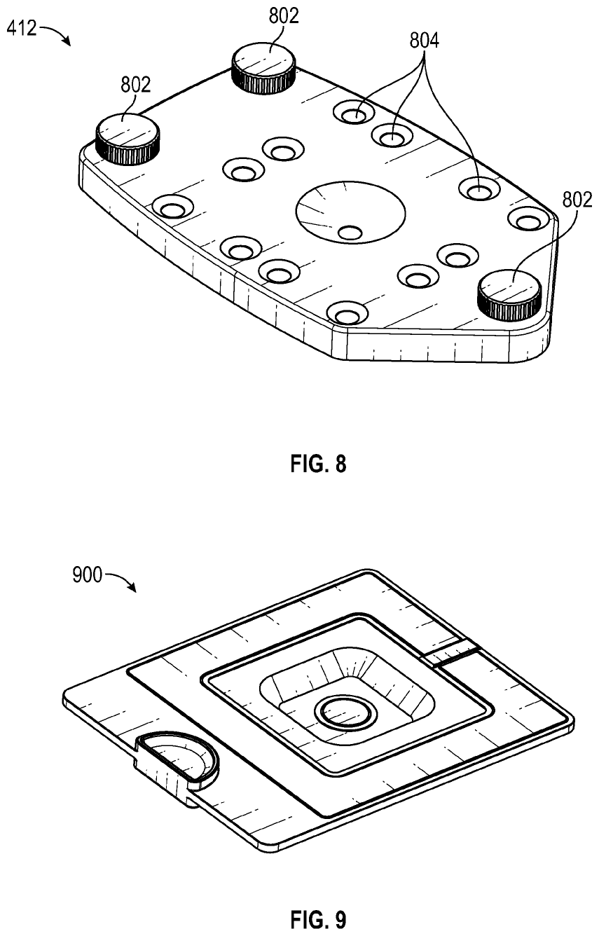

FIG. 8 is a perspective view of marker 412. Marker 412 comprises pins or screws 802 to facilitate accurate and stable placement of the marker 412 at the marker holder 410 on the X-ray calibration jig 38. In accordance with several implementations, location accuracy of the marker 412 is achieved in a manner such that the marker angular deviation does not exceed 0.2 degrees, 0.18 degrees, 0.16 degrees, 0.15 degrees, 0.14 degrees and/or less than 0.15 mm (root mean square value) omnidirectionally from its nominal or theoretical position. Marker 412 further includes reflective elements 804 arranged on a plane for reflecting infrared light. Marker 412 may also include a reflective element (e.g., the central reflective element) positioned on a different plane spaced apart from the plane on which the other reflective elements 804 are located. In some embodiments, marker 412 comprises three pins or screws 802. In some embodiments, marker 412 can comprise greater or fewer than three pins or screws 802.

FIG. 9 shows a perspective view of an inner, or lower, portion or component 900 of marker 600 shown in FIG. 6. The lower portion or component 900 of marker 600 includes reflective material that forms the reflective elements created by the pattern of openings in an upper portion or component of the marker, as shown in FIG. 6, so as to facilitate imaging and tracking by an infrared camera or sensor of the head-mounted units of FIGS. 1, 2A and 2B. The central well of the lower portion or component is on a different, second plane than the first plane of the outer perimeter. The central well also includes reflective material. In various implementations, marker 600 is a disposable marker or a reusable marker. As described above, the marker 600 may comprise a snap-fit attachment mechanism.

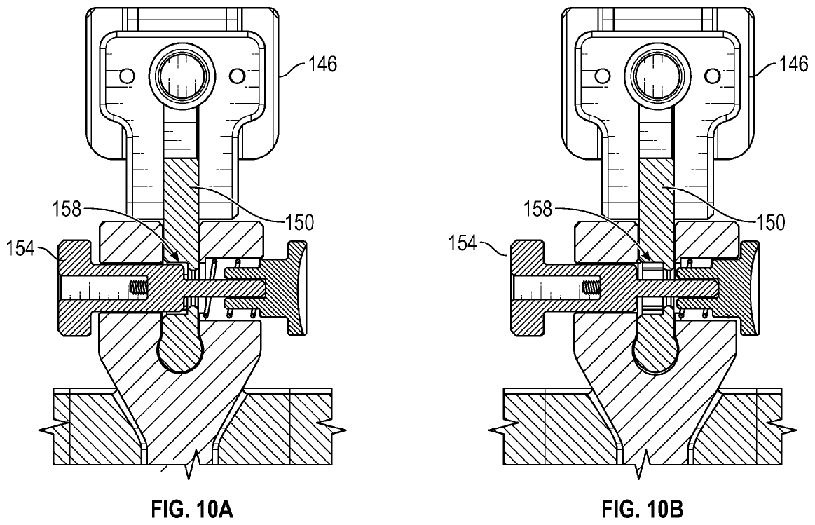

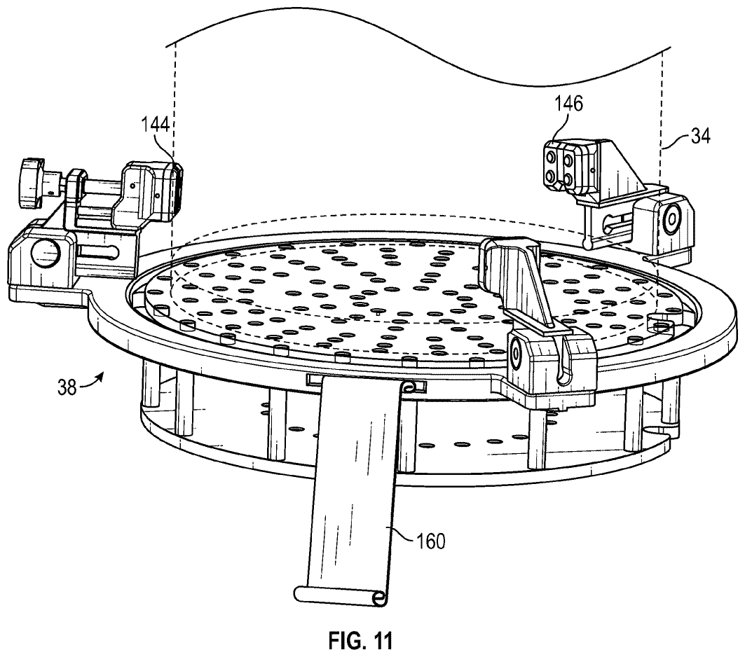

FIGS. 10A and 10B are schematic rear views of the sliding mechanism on which pad 146 is mounted, in accordance with an embodiment of the disclosure. In FIG. 10A, lock button 154 is actuated by inserting the lock button 154 into a detent 158 in slide 150, thus preventing movement of the pad 146. In FIG. 10B, lock button 154 is actuated so that pad 146 is able to shift radially. Similar or alternative sliding and locking mechanisms may be used in the other illustrated embodiments as well.

FIG. 11 is a schematic pictorial illustration showing details of X-ray calibration jig 38, in accordance with embodiments of the disclosure. Jig 38 is similar in design to the jig shown in FIGS. 10A and 10B, with the addition of a safety strap 160, which fastens around the back of X-ray detector 34 and secures the rings of the jig 38 to the X-ray detector 34 to prevent accidental release of the jig 38. Multiple safety straps may be used attached at various locations around a circumference of the jig 38. The jig 38 of FIG. 11 may incorporate any of the structural or operational features of the jigs shown and described in connection with FIGS. 10A and 10B or other previous figures.

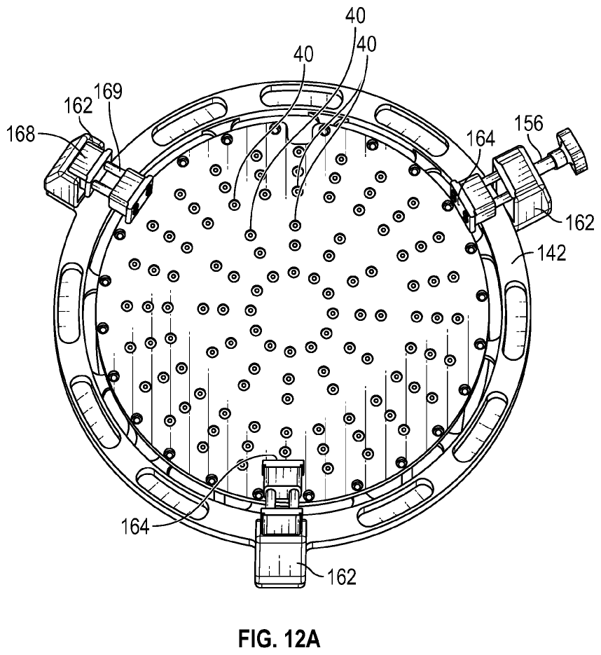

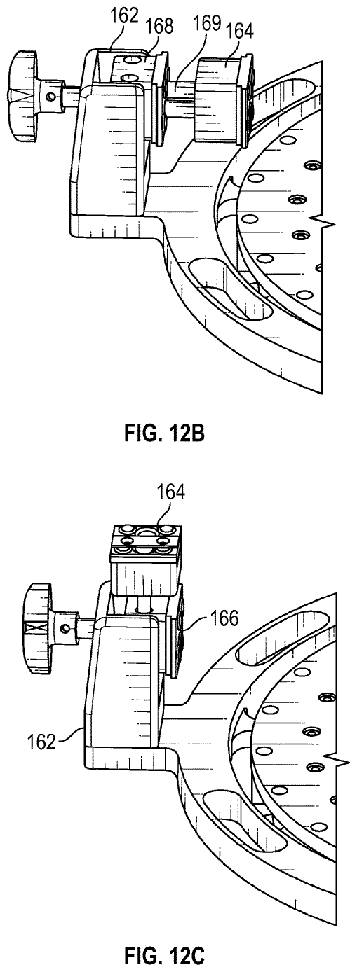

FIGS. 12A, 12B, and 12C are schematic pictorial illustrations of an X-ray calibration jig 38 with an alternative mechanism 162 for securing the jig 38 to X-ray detector 34, in accordance with an embodiment of the disclosure. FIG. 12A is a top view showing upper ring 142 of the jig with three mechanisms 162 of this sort distributed around the periphery of the upper ring 142; while FIGS. 12B and 12C show details of mechanism 162 in two different operating configurations, for use with X-ray detectors (e.g., of C-arms) of different sizes (e.g., diameters).

Each mechanism 162 comprises two pads 164 and 166, which are mounted on a base 168. In FIGS. 12A and 12B, pads 164 are rotated downward, so that pads 164 extend inward on extension arms 169 to engage a camera of small diameter. In FIG. 12C, pads 164 are rotated upward, moving pads 164 out of the way, so that pads 166 (without extension arms) will engage a camera of larger diameter. The jigs of FIGS. 12A-12C may incorporate any of the structural or operational features of the jigs shown and described in connection with FIG. 7, 11 or other previous figures.

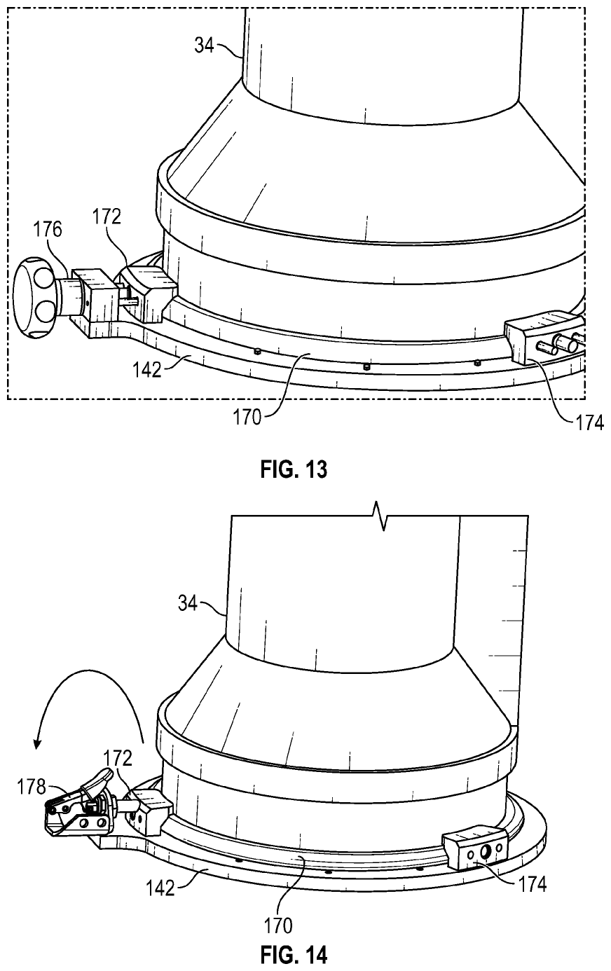

FIG. 13 is a schematic pictorial illustration showing a part of an X-ray calibration jig 38 that fits over a peripheral lip 170 of X-ray detector 34, in accordance with an embodiment of the disclosure. Not all fluoroscopes have such a lip, but when lip 170 is present it can be used advantageously to hold jig 38 in place. For this purpose, the X-ray calibration jig comprises multiple anchors 172, 174, which are disposed around the circumference of ring 142 and engage lip 170. An adjustment knob 176 can be turned in order to shift anchor 172 in the radial direction so as to engage and lock over lip 170 and thus hold the jig firmly in place.

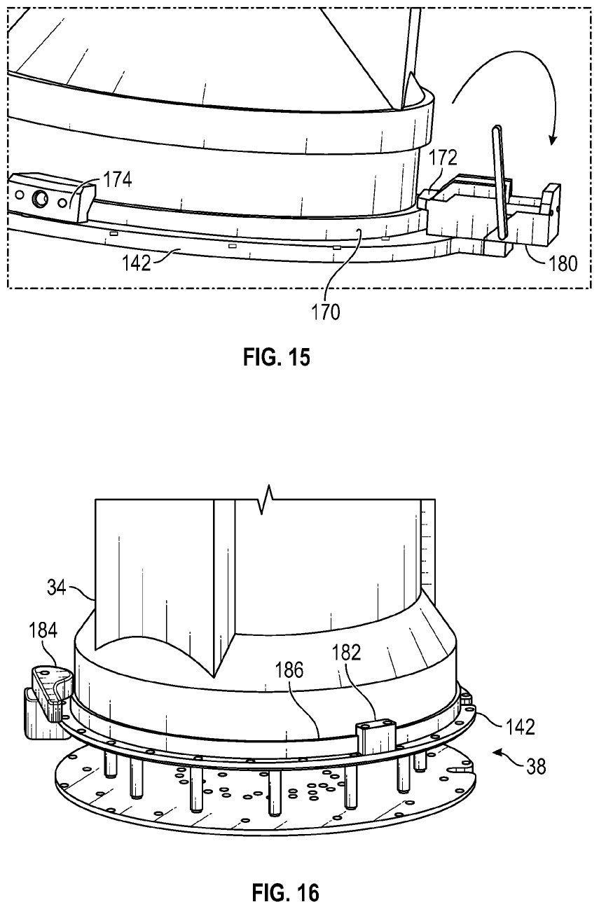

FIGS. 14 and 15 are schematic pictorial illustrations showing alternative mechanisms for shifting anchor 172 radially to lock over lip 170, in accordance with further embodiments of the disclosure. In FIG. 14, a linear toggle 178 is pressed inward to lock anchor 172 over lip 170 and pulled outward to release the anchor. In FIG. 15, a spring-based latch 180 locks and releases anchor 172.

FIG. 16 is a schematic pictorial illustration showing X-ray calibration jig 38 with a mounting arrangement that fits over a peripheral lip 186 of X-ray detector 34, in accordance with another embodiment of the disclosure. In this embodiment, X-ray calibration jig 38 comprises multiple anchors 182, which are disposed around the circumference of ring 142 and engage lip 186. An eccentric locking knob 184 is turned to press against the surface of X-ray detector 34 and thus hold the jig firmly in place.

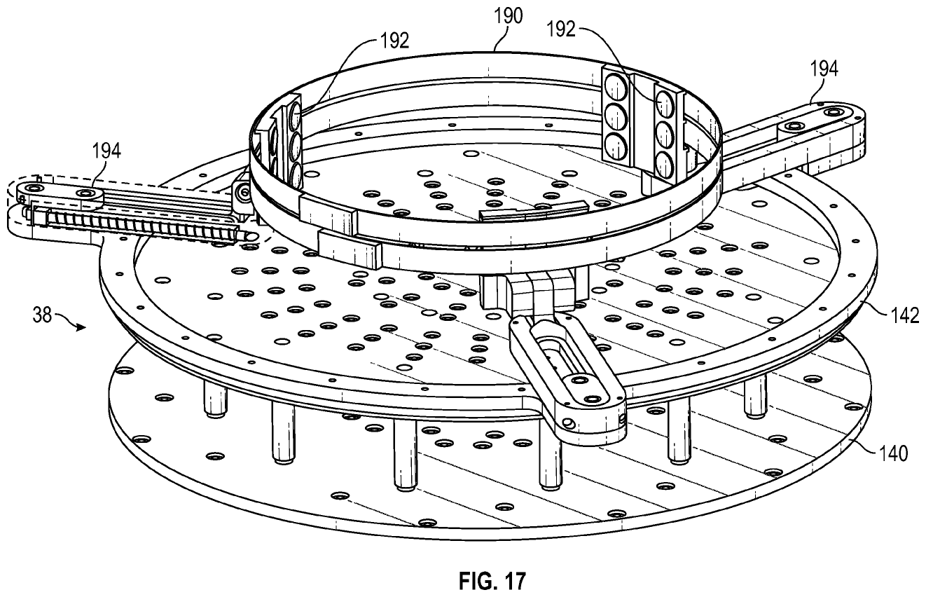

FIG. 17 is a schematic pictorial illustration showing X-ray calibration jig 38 with a mounting arrangement based on flexible bands 190, which clamp around a peripheral surface of an X-ray detector, in accordance with an embodiment of the disclosure. Elastomer pads 192 press against and grip the outer surface of the X-ray detector 34. Pads 192 are mounted on slides 194, which enable the pads to shift in a radial direction so as to engage X-ray detectors of different diameters. Bands 190 secure pads 192 in place to ensure that jig 38 remains firmly attached to the X-ray detector 34.

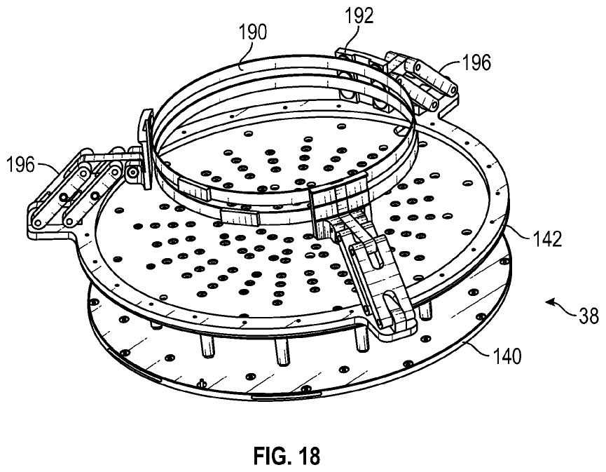

FIG. 18 is a schematic pictorial illustration showing X-ray calibration jig 38 with a mounting arrangement based on vertical parallelogram mechanisms 196, which are locked by flexible bands 190, in accordance with another embodiment of the disclosure. In this embodiment, vertical parallelogram mechanisms 196 press pads 192 inward against the surface of the X-ray detector 34.

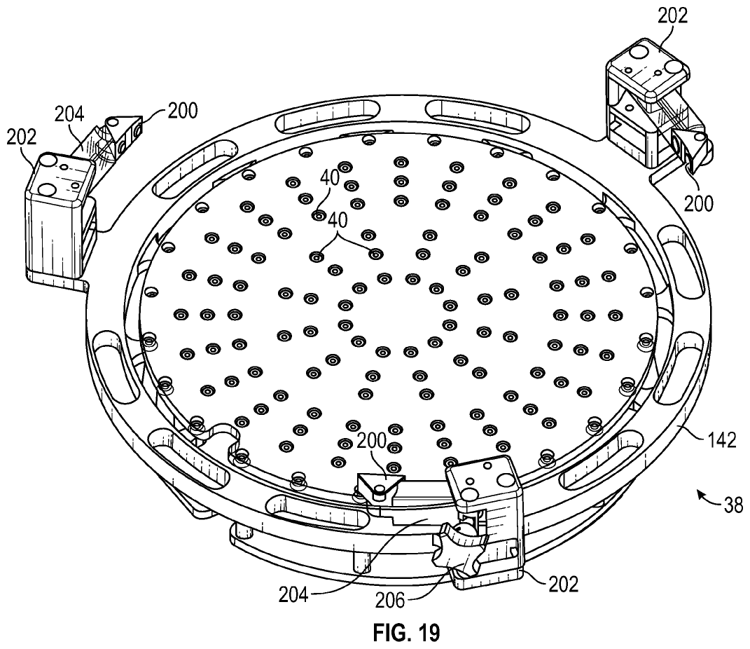

FIG. 19 is a schematic pictorial illustration showing X-ray calibration jig 38 with a mounting arrangement based on radial locking mechanisms 202, in accordance with an embodiment of the disclosure. Each locking mechanism 202 comprises an elastomer pad 200, which rotates on a respective arm 204 to engage the outer surface of an X-ray detector, so that jig 38 can be used with cameras of different sizes. A locking knob 206 can be turned to provide an additional adjustment range and secure the jig in place.

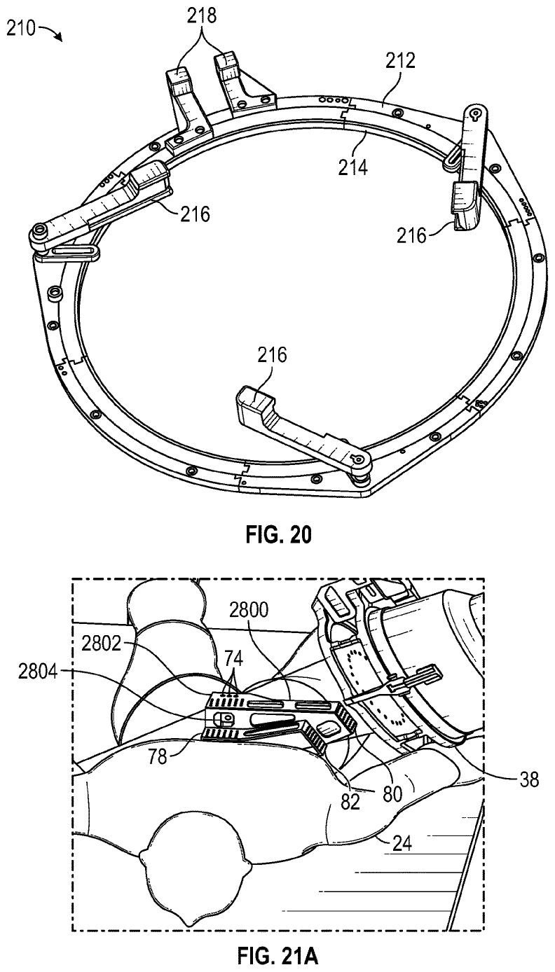

FIG. 20 is a schematic pictorial illustration showing a self-centering mechanism 210 for X-ray calibration jig 38, in accordance with an embodiment of the disclosure. Mechanism 210 comprises an outer ring 212 and an inner ring 214, which can be attached to or can take the place of upper ring 142 in jig 38. Two knobs 218, connected together by a lead screw (not shown), are attached respectively to outer ring 212 and inner ring 214. Moving knobs 218 together or apart causes inner ring 214 to rotate relative to outer ring 212. Pads 216 are mounted on outer ring 212 and rotate inward and outward in response to the relative rotation between inner ring 214 and outer ring 212. Thus, manipulation of knobs 218 shifts all of pads 216 together so as to center ring 212 relative to the peripheral surface of the X-ray detector 34, and thus to center the entire calibration jig 38.

This approach may be useful in ensuring that the jig 38 will be centered correctly regardless of the camera size. Flexible bands (for example as shown in FIG. 18), may be positioned around pads 216 to ensure that the connection of the jig 38 to the detector 34 is secure.

In some embodiments, the system comprises various features that are present as single features (as opposed to multiple features). For example, in one embodiment, the system includes a single camera, a single jig, a single marker, a single ring, a single anchor, a single pad etc. Multiple features or components are provided in alternate embodiments.

### Registration Targets or Markers

As explained above, although registration target 46 in the embodiment shown in FIG. 1 comprises a radiopaque pattern and an optical pattern, in other embodiments the registration target comprises only a radiopaque pattern in a predefined spatial relation to the patient marker. In some embodiments, an optical marker, such as optical marker 42 is positioned in a predefined spatial relation to the registration radiopaque pattern, such as beads 40. In some embodiments, the X-ray images captured by fluoroscope 30 contain both the X-ray calibration pattern on jig 38 and the radiopaque pattern of the registration target. In some embodiments, the registration target is fixed in the location of the patient marker during acquisition of the X-ray images for purposes of calibration and registration and the registration target is then removed so that the patient marker may be visible during the surgery. In other embodiments, the registration target is fixed to the patient marker at a selected distance from the optical pattern of the patient marker and thus may remain in place during the surgery. The radiopaque pattern of the registration target typically comprises radiopaque elements, such as beads, which are disposed in multiple different planes. Examples of registration targets with such features are shown in the figures that follow.

FIGS. 21A and 21B are schematic pictorial views showing a registration target 2800 attached by a pin 2804 to the back of patient 24, in accordance with an embodiment of the disclosure. Registration target 2800 comprises patterns of radiopaque elements, such as metal beads 74, which may be arranged in multiple planes: two parallel planes 2802 and 78, which are approximately horizontal and are offset axially relative to one another along a normal to the planes; and two parallel oblique planes 80 and 82, which are similarly offset axially relative to one another. Although the patterns of beads 74 in all of planes 2802, 78, 80 and 82 are identical in FIGS. 21A and 21B, in alternative embodiments the patterns in some or all of the planes may be different from one another. Furthermore, although registration target 2800 in FIGS. 21A and 21B includes two pairs of parallel planes, in alternative embodiments, the planes need not be parallel.

To compute the transformation between fluoroscope 30 and registration target 2800, X-ray detector 34 may capture fluoroscopic images from two different angles relative to the patient's body, for example one anteroposterior (AP) image and one lateral (LT) image. (FIGS. 21A and 21B show X-ray detector positioned to capture the LT image.) Alternatively a single fluoroscopic image may be sufficient to compute the transformation between fluoroscope 30 and registration target 2800. Each image includes both the patterns of beads 2400 in two different planes of registration target 2800 and beads 40 in the pattern on calibration jig 38. For example, the LT image includes the patterns in planes 80 and 82, while the AP image includes the patterns in planes 2802 and 78. After calibrating X-ray detector 34 to compensate for distortion, as described above, processor 50 may compare the locations of the patterns of beads 40 and 2400 in the fluoroscopic image to the known geometrical layouts of the patterns and thus compute a geometrical transformation between the frames of reference of fluoroscope 30 and of registration target 2800. The transformation comprises coefficients of 3D translation and rotation between the two frames of reference. The coefficient values are optimized to achieve the best fit to the relative positions of the patterns of beads 40 and 2400.

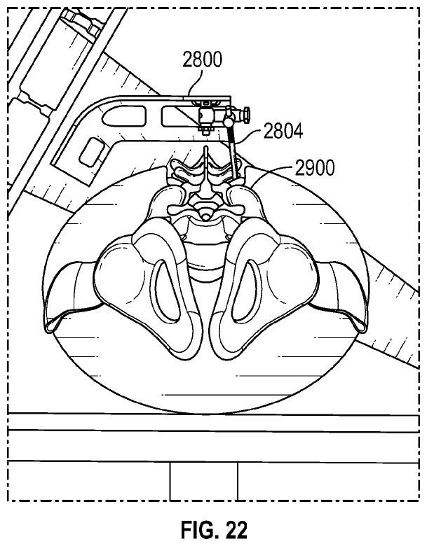

FIG. 22 is a schematic sectional illustration showing another configuration of registration target 2800 attached to pin 2804, in accordance with an embodiment of the disclosure. In this example, pin 2804 has been surgically inserted into an iliac crest 2900 of the patient 24 and thus provides a stable platform for registration target 2800, which is stationary relative to the patient's skeleton. As in the preceding embodiment, registration target 2800 comprises patterns of radiopaque elements, such as metal beads 2400, which may be arranged in multiple planes: two parallel horizontal planes which are offset axially relative to one another along a normal to the planes; and two parallel oblique planes, which are similarly offset axially relative to one another. In accordance with several embodiments, after the fluoroscopic calibration procedure is completed, registration target 2800 is removed from pin 2804, and an optical patient marker is attached in its place.

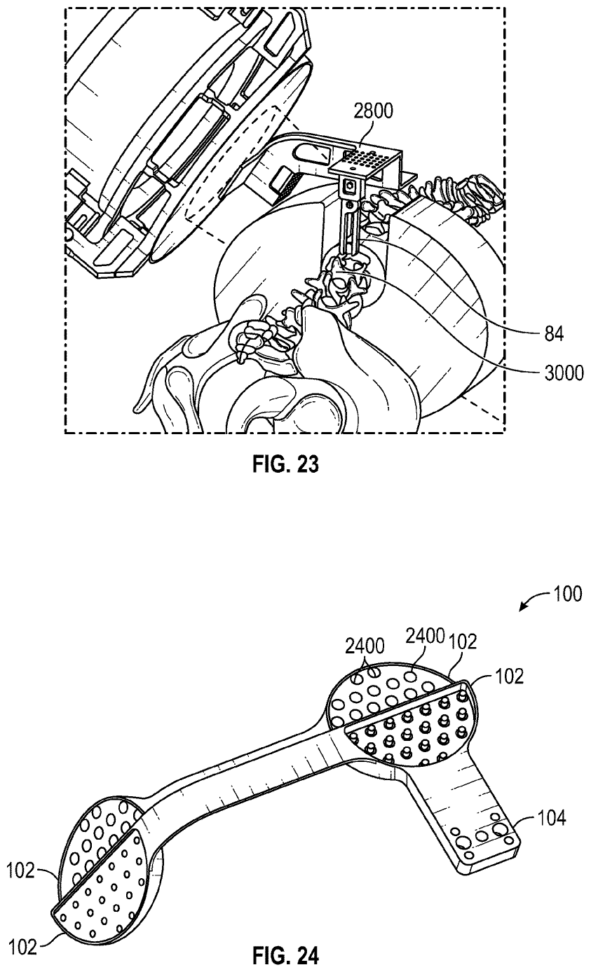

FIG. 23 is a schematic sectional illustration showing registration target 2800 attached to a clamp 84, in accordance with an alternative embodiment of the disclosure. Clamp 84 is fastened over a spinous process 3000, which similarly provides a stable platform. In this case, too, registration target 2800 may be removed from clamp 84 after the fluoroscopic calibration procedure is completed and replaced by an optical patient marker.

FIG. 24 is a schematic pictorial illustration of a registration target 100, in accordance with another embodiment of the disclosure. As in the preceding embodiment, target 100 comprise radiopaque beads 2400 arranged in predefined patterns in multiple different planes 102, while each two parallel planes may be imaged from a different angle relative to the patient's body (e.g., from AP and LT angles). Mounting holes 104 enable registration target 100 to be fixed stably, in a known orientation, to the pin or clamp that will subsequently hold the optical patient marker.

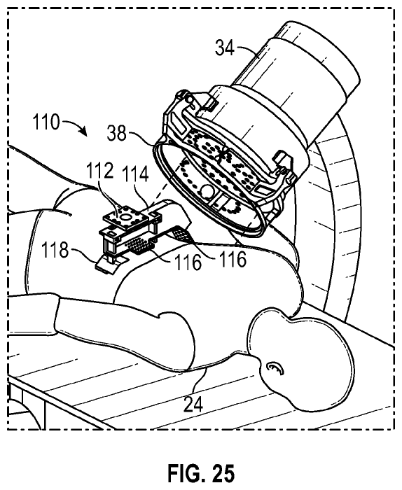

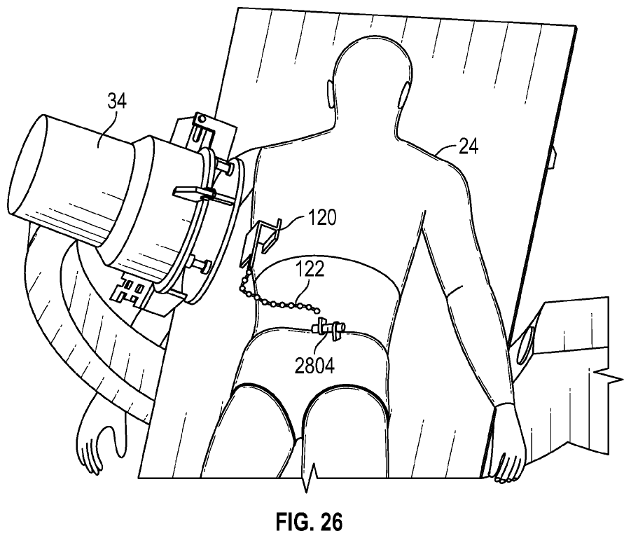

FIG. 25 is a schematic pictorial illustration showing a multimodal target 110 attached to the back of patient 24, in accordance with another embodiment of the disclosure. Target 110 comprises an optical pattern 112, which allows the registration of the X-ray frame of reference with the optical frame of reference, and X-ray patterns 116 of radiopaque elements, serving as the registration target. Both optical pattern 112 and X-ray patterns 116 are fixed to a frame 114, in a predefined spatial relationship. Frame 114 includes a mount 118, which is fixed to the body surface of patient 24, for example using a suitable adhesive.

In operation, fluoroscope 30 captures images of patient 24 including target 110 from two different angles, as explained above. The images contain both X-ray patterns 116 and the calibration pattern on jig 38 and are thus used by processor 50 (FIG. 1) both in calibrating the fluoroscope and in registering the fluoroscope with target 110. An image of both the patient marker (not shown) and optical pattern 112 may be captured by camera 48. Because the spatial relationship between optical pattern 112 and X-ray patterns 116 is known and fixed, the transformation between the X-ray and optical frames of references may be computed based on the geometry of target 110 and the image of optical pattern 112 and the patient marker. Thus, processor is able to use the X-ray and optical images in registering the fluoroscope with the body of the patient 24. Target 110 may be then removed from patient 24.

FIG. 26 is a schematic pictorial illustration showing a multimodal target 120 attached to the back of patient 24, in accordance with another embodiment of the disclosure. Target 120 is connected via a flexible extension arm 122 to the skeleton of patient 24, for example by pin or clamp 2804. Alternatively, extension arm 122 may be attached to the operating table or to another stable anchoring point in the vicinity of patient 24. Extension arm 122 has a geometrical configuration that can be adjusted and then locked in place. This feature allows multiple degrees of freedom in placing target 120. Arm 122 may be shifted out of the surgical field or removed after the registration procedure has been completed. The flexible extension arm feature may be incorporated into any of the other registration target embodiments described herein.

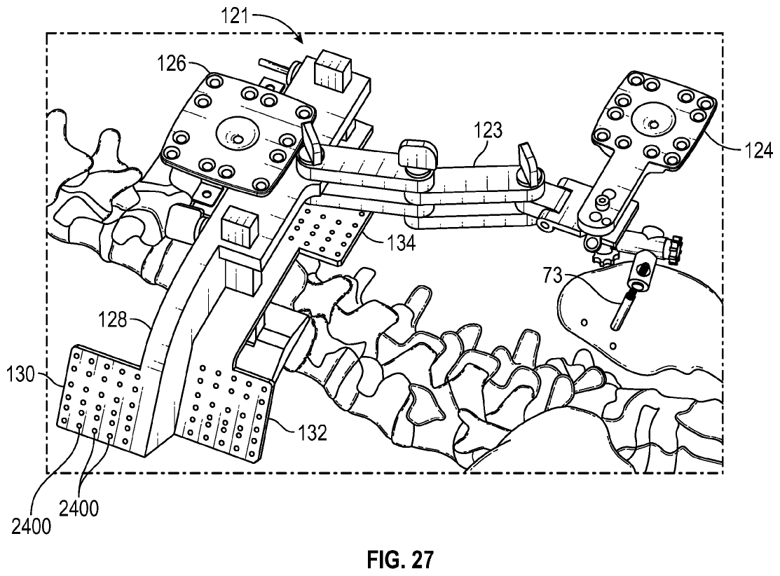

FIG. 27 is schematic pictorial illustration showing a multimodal target 121, in accordance with an embodiment of the disclosure. Multimodal target 121 includes an optical pattern 126. A patient marker 124 comprising an optical pattern is mounted to the patient via pin 73. Camera 48 may capture images of both optical markers 124 and 126 to determine the location of multimodal target 121 with respect to patient marker 124. The optical pattern of patient marker 124 indicates the location of pin 73, while pattern 126 indicates the location of a registration target 128, which is displaced from pin 73 (or by another stable anchoring point) by a rigid extension arm 123. Extension arm 123 has a geometrical configuration that can be adjusted and then locked in place. Registration target 128 comprises multiple X-ray patterns 130, 132, 134 of radiopaque beads 2400, which are located in different planes. In accordance with several embodiments, processor 50 processes fluoroscopic images containing registration target 128, along with optical images of the optical pattern of patient marker 124 and of optical pattern 126, in order to register the location and orientation of fluoroscope 30 relative to the body of patient 24. More than two optical patterns may be used in other embodiments. The multiple X-ray patterns may include two, three, four, or more than four patterns.

### Registration

In image-guided surgery, it is important that the anatomical images displayed to the surgeon or other clinical professional to provide guidance and/or facilitate navigation (e.g., of medical tools and instruments) within the patient body, correspond to the current anatomy of the patient (e.g., pose and/or structure). In addition, for this sort of AR to be clinically useful, it can be important that the overlaid 3D images be properly registered with the actual anatomical structures in the body. When the 3D images are acquired during the operation, for example using an intraoperative 3D medical imaging scanner, such as a CT or MRI scanner, proper registration will be maintained as long as the patient is stationary. In most surgeries, however, the 3D images are acquired before the surgery, in a different room, and, for example, the patient's pose on the operating table is often different from that in the tomographic image. Three-dimensional images acquired preoperatively typically will not have a reference (e.g., a fiducial marker) which will allow the registration of the preoperative 3D images with the patient anatomy at the time of the operation. In addition, there may be a change in the relative location of vertebrae in the patient's spine between the time the 3D images were acquired and the time of the operation. Such a change may be due to a change in the patient's pose, an insertion of an implant, or any other reason. In the case of spinal surgery, the surgeon typically uses a fluoroscope in the operating room to acquire 2D images during surgery and uses these 2D images for guidance during the surgery, while viewing pre-acquired medical images (e.g., tomographic images or volumetric images) of the spine offline.

Embodiments of the disclosure that are described herein provide methods, systems and computer software products that can be used to register a pre-acquired 3D medical image (e.g., tomographic image or MR image) with intraoperative 2D fluoroscopic images. In the disclosed methods, systems and computer software products, the 3D image is segmented into multiple 3D segments, each containing a respective one of the vertebrae or other bony portions. Each of these 3D segments is registered with a respective vertebra or other bony portion in the fluoroscopic images. Specifically, the respective location and orientation of each 3D segment may be adjusted to match the respective vertebra or other bony portion in the fluoroscopic images and thus to account for changes in the relative location of vertebrae or other bony portions (e.g., due to a change in the patient's pose on the operating table relative to the pose in the 3D image). In the disclosed embodiments, two fluoroscopic images, captured from different angles, are used together in this registration process; but alternatively, a larger number of fluoroscopic images (e.g., three, four or more than four images) may be used.

In the context of spine surgery, when the registration process is complete, an image of the spine comprising the registered 3D segments is presented on a display, for example by overlaying an AR image of the registered 3D segments on the back of the patient to generate an AR view (e.g., to facilitate AR-assisted navigation). In some embodiments, to ensure proper registration between the AR image and the patient's body, the frame of reference of the 2D fluoroscopic images is calibrated relative to the patient's body (e.g., a portion of a back of the patient corresponding to a target treatment area of the spine), for example using calibration markers as described hereinbelow. This calibrated frame of reference may then be applied to the 3D image so that the vertebrae or other bony portions in the registered 3D segments are aligned properly with the spine of the patient.

### Registration of Tomographic and Fluoroscopic Images

For purposes of AR image display, it may be important that the frame of reference of the images captured by fluoroscope 30 be calibrated relative to the physical frame of reference of the body of patient 24. There are two aspects to this calibration: (1) correction of distortion in images captured by fluoroscope 30 itself, and (2) registration of fluoroscope 30 with the patient's body. For these purposes, according to the example system illustrated in FIG. 1, a calibration jig (e.g., ring adapter) 38 is fitted over the X-ray detector, in this case X-ray detector 34. In the pictured embodiment, the calibration jig (e.g., ring) comprises an array of X-ray opaque beads 40 in a predefined pattern, along with an optical marker 42 in a known position and orientation relative to the pattern of beads 40. The bead pattern appears in the fluoroscopic images captured by X-ray detector 34. In some embodiments, a processor 50 receives and processes these images in order to correct X-ray image distortion and determines the location and orientation of the optical axis of fluoroscope 30.

In accordance with several embodiments, optical marker 42 is used in conjunction with an optical marker on the body of patient 24 in registering the optical axis of fluoroscope 30 with the body. For example, surgeon 22 may attach a marker 44 to the patient's spine, using a suitable bone clamp or percutaneous pin. Additionally, or alternatively, surgeon 22 may fix a marker 46 to the patient's body surface (e.g. skin). The marker 46 may be fixed via a self-adhesive backing on the marker 46 or via a separate adhesive (e.g., adhesive tape or glue). In some embodiments, a camera 48 (e.g., infrared camera or other optical camera) captures images including both optical marker 42 on calibration ring 38 and marker 44 or marker 46 attached to patient 24. Although camera 48 in FIGS. 1 and 2A and 2B is mounted on head-mounted AR display unit 28, these images may alternatively be captured by a suitable camera mounted elsewhere on the head or body of surgeon 22 or mounted elsewhere in the operating room or diagnostic imaging room (e.g., in a stationary manner). In some embodiments, processor 50 processes the images in order to calculate the location and orientation of fluoroscope 30 relative to the patient's body and thus to calibrate the fluoroscopic frame of reference relative to the frame of reference of the body.

This calibration process is described herein, as are descriptions of other configurations of the calibration ring and optical markers that can be used in place of the configuration that is shown, by way of example, in FIG. 1. Alternatively, other devices and methods may be used in calibrating the fluoroscopic frame of reference to the patient's body for purposes of AR display of 3D medical image data (e.g., tomographic image data) and are considered to be within the scope of the present disclosure.

In addition to receiving 2D X-ray images from fluoroscope 30, processor 50 is configured to receive 3D medical images of patient 24, for example CT or MRI images, typically acquired prior to the surgery or other medical intervention, and store these 3D images in a memory 52. Processor 50 is configured to segment the 3D images and register the 3D segments with respective vertebrae in the 2D fluoroscopic images. The processor 50 is then configured to present an image of the spine comprising the registered 3D segments on head-mounted AR display unit 28, such that the vertebrae in the 3D images are aligned with the actual vertebrae of the patient's spine. Details of this process are described with reference to the figures that follow. Alternatively, the registered 3D segments may be presented on a different sort of display, for example on an AR display that is mounted on patient 24 or on operating table 26 above the surgical site or another local display and/or on a remote display device. Additionally, or alternatively, the registered 3D segments may be presented on a non-AR display, such as a display of a workstation or of a hand-held computer.

In accordance with several embodiments, processor 50 comprises a general-purpose computer processor, which is programmed in software to carry out the functions of calibration, registration, and display that are described herein. This software (e.g., executable program instructions) may be stored on tangible, non-transitory computer-readable media, such as optical, magnetic, or electronic memory media. Additionally or alternatively, at least some of the functions of processor 50 may be carried out using special-purpose computing hardware, such as a graphics processing unit (GPU). Processor 50 may include one or more processors. Processor 50 may be located in a workstation and/or in head-mounted AR display unit 28.

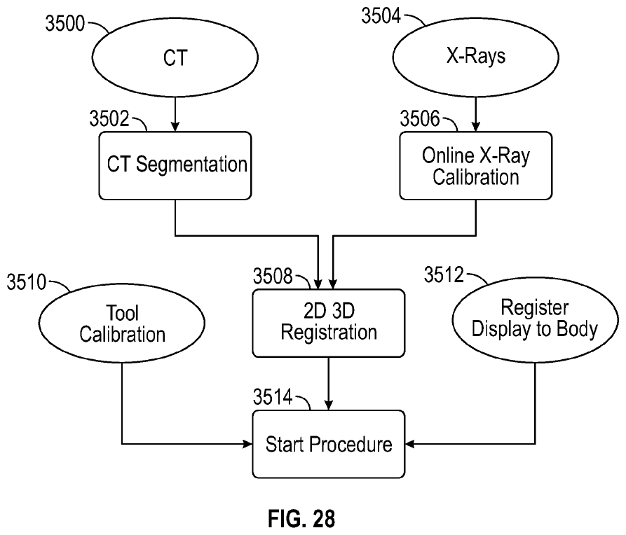

FIG. 28 is a flow chart that schematically illustrates a method for generating an AR display based on registration of 3D and 2D images, in accordance with an embodiment of the disclosure. The method is described here, for the sake of concreteness and clarity, with reference to system 20 (FIG. 1), assuming processor 50 has received a 3D CT image of the spine of patient 24 and receives two X-ray images from fluoroscope 30, captured with X-ray detector 34 at two different angles. Alternatively, the present method may be applied, mutatis mutandis, using other sorts of medical images, such as MRI images or other tomographic images that have been processed to segment bones from soft tissue, as well as using larger or smaller numbers of 2D X-ray images. The principles of this method may also be applied in generating images of other bones in the patient's skeleton (e.g., hip bones, pelvic bones, leg bones, arm bones, ankle bones, foot bones, shoulder bones, cranial bones, oral and maxillofacial bones, sacroiliac joints, etc.) With respect to the spine, the vertebrae may include lumbar vertebrae, sacral vertebrae, cervical vertebrae, and/or thoracic vertebrae, or other bony structures, portions, elements or components.

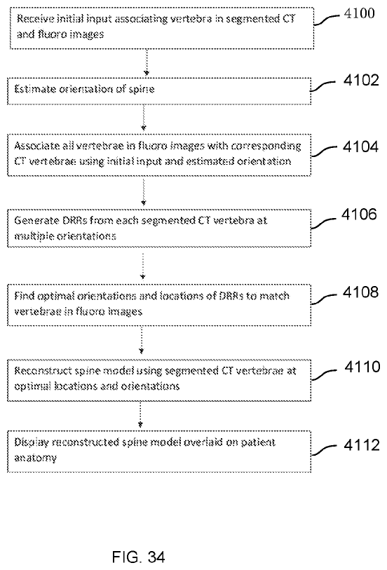

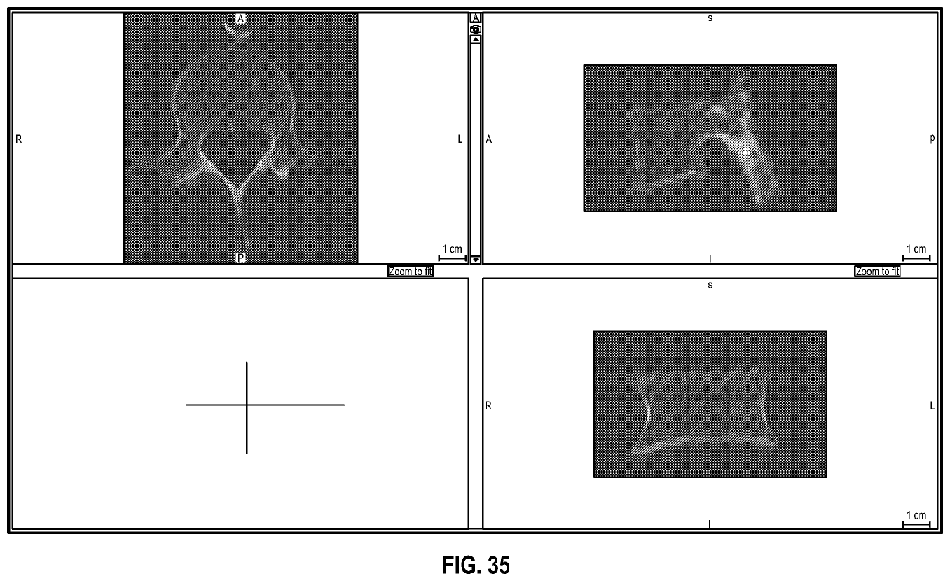

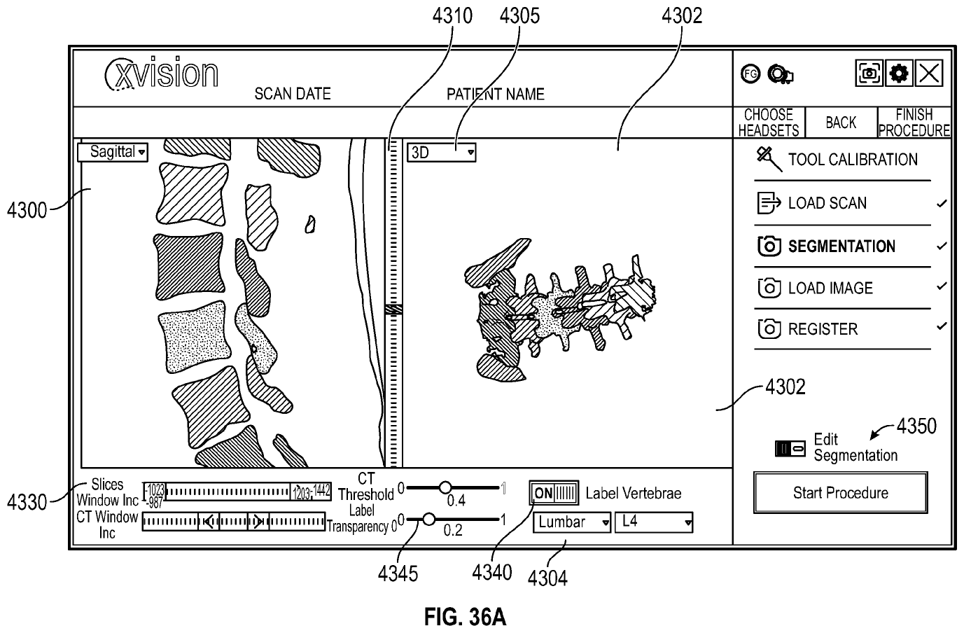

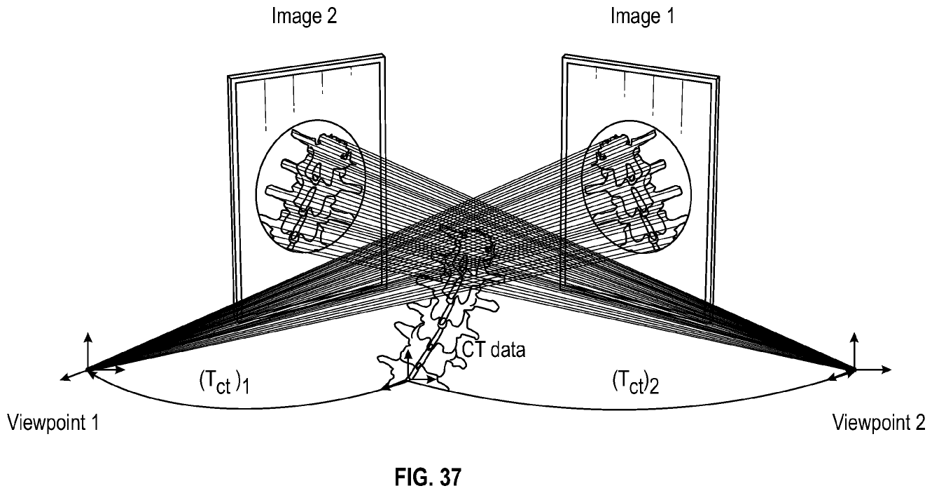

As explained above and illustrated in FIG. 28, both the 3D CT images and the 2D X-ray images are preprocessed (at Block 3500 and 3504) to enable registration between the images and display of the vertebrae or other bony structures from the CT image (e.g., on displays 60) in alignment with the patient's spine. As noted above, processor 50 is configured to, upon execution of computer-readable program instructions, calibrate the X-ray images online at block 3506 to correct distortion and register fluoroscope 30 with the body of patient 24, as described herein. The CT image is segmented into multiple 3D segments at step 3502, each containing a respective one of the vertebrae and/or sacrum and/or ilium and/or other bony structures, as described further hereinbelow with reference to FIG. 35. The 3D segments are then registered (Block 3508) with the calibrated 2D fluoroscopic images, as described hereinbelow with reference to FIGS. 34 and 37.

In accordance with several embodiments, to present the AR images on displays 60 in alignment with the patient's anatomy, processor 50 registers display unit 28 (or display unit 70, correspondingly) with the patient's body using images of marker 44 and/or marker 46 (Block 3512), as explained above. Surgical tools (not shown), such as drills, introducers, cannulas, curettes, stylets, screwdrivers, inserters, etc., may be provided with similar sorts of markers (directly or indirectly), to enable processor 50 to calibrate and register their positions (Block 3510), as well, and thus to incorporate virtual images of the tools in the AR displays, as well. Once all elements have been calibrated and registered in this manner, surgeon 22 can carry out the desired surgical or other medical procedure (Block 3514) with the assistance of AR images of the patient's vertebrae or other bony structures or portions presented on displays 60. The method of FIG. 28 will apply, mutatis mutandis, when a non-AR system is used. When a non-AR system is used, the step of registering the display to the body should be omitted.

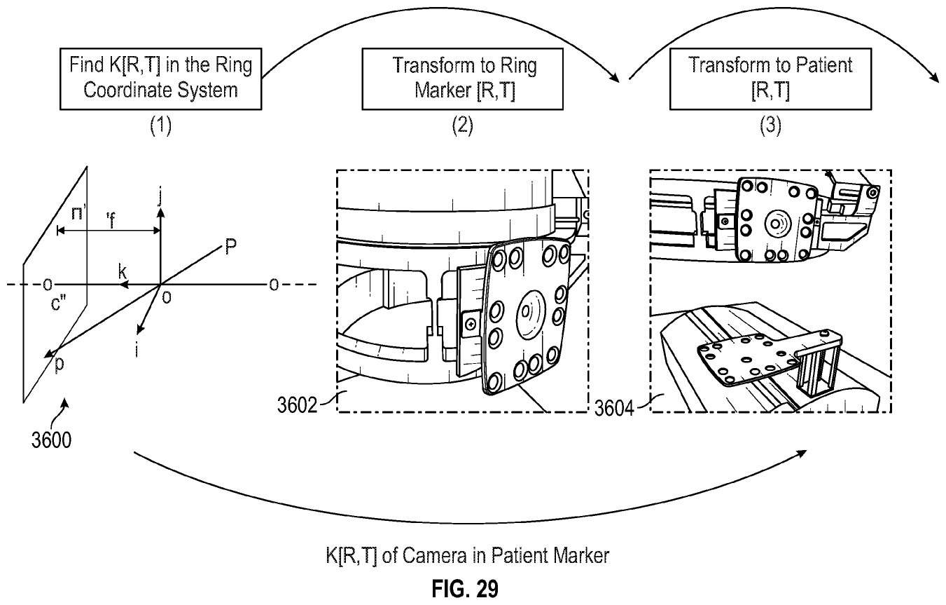

FIG. 29 is a schematic illustration of a calibration process, in accordance with an embodiment of the disclosure. In this calibration process, parameter information pertaining to the imaging system is calculated to determine a 3D to 2D mapping (e.g., mapping or finding the correspondence for each 3D voxel from a 3D scan acquired pre-operatively to a 2D pixel from a 2D fluoroscopic image acquired intraoperatively as illustrated by 3600 in FIG. 29). To map each 3D voxel to a 2D pixel, intrinsic and extrinsic parameters of a fluoroscope are estimated using a mathematical relationship that considers n points P₁, . . . , Pₙ with known coordinates and known positions in the image comprising points p₁, . . . , pₙ.

$$p = K [ \begin{matrix}R & T\end{matrix} ] {P}_{w}$$

K[R T] represents the transformation matrix that maps each 3D voxel to a 2D pixel. K represents the intrinsic parameters of the fluoroscope or other device while [R T] represents the extrinsic parameters, where R is a rotational matrix and T is a translational matrix.

$$K = [ \begin{matrix}α & s & {c}_{x} \\ 0 & β & {c}_{y} \\ 0 & 0 & 1\end{matrix} ]$$

$$R = [ \begin{matrix}- & {r}_{1} & - \\ - & {r}_{2} & - \\ - & {r}_{3} & -\end{matrix} ]$$

$$T = [ \begin{matrix}{t}_{x} \\ {t}_{y} \\ {t}_{z}\end{matrix} ]$$

The calibration process includes finding the transformation matrix K[R,T] in the X-ray calibration jig, or ring, coordinate system, transforming to the jig or ring marker's coordinate system 3602 (e.g., marker 412, marker 600 attached to the jig 38-C-marker [R,T]), and then transforming to the patient's coordinate system 3604 (e.g., Patient [R,T]). The calibration process results in obtaining the transformation matrix K[R, T] of the camera (e.g., fluoroscope) in the patient marker's coordinate system.

In accordance with several implementations, the goals of the X-ray calibration process are to find the intrinsic parameters of the fluoroscope or other imaging device (e.g., focal length and principal point) and the extrinsic parameters (translation and orientation) in relation to the patient marker coordinate system. In order to calculate the intrinsic parameters, a double-layer calibration jig having plates with different fiducial element or bead patterns in each layer is attached on the detector of the C-arm or other fluoroscope or imaging device. First, the beads on an upper layer which appear in the image are detected and then each bead is associated to its pattern. Now, the system includes a batch of correspondences of 3D-2D points (3D points in the calibration jig or C-arm coordinate system and 2D points in the image) and the intrinsic parameters can be calculated.

In accordance with several implementations, in order to calculate the extrinsic parameters, there are several different options. One option is to connect a registration marker on a clamp in a fixed offset to the patient marker. The registration marker may be captured and may appear in the X-ray image and with a similar process (e.g., detection and association), the translation and orientation of the X-ray detector can be calculated in the registration marker's coordinate system. Then, the system can transform from the registration marker's coordinate system to patient marker's coordinate system based on the mechanical known offset.

A second option is to connect an optical marker on the calibration jig 38 in a fixed offset to the jig's coordinate system, as shown in FIGS. 1-7. After capturing one or more X-ray, or fluoroscopic, images, the head-mounted unit (e.g., infrared camera or other imaging device) may capture images of the patient marker and the optical marker. In accordance with several implementations, marker position must be captured for each X-ray, or fluoroscopic, image before moving on to the next X-ray. Using those images, the transformation between the optical marker and the patient marker can be calculated. Since the position and orientation of the optical marker in the jig's coordinate system is known, the transformation between the jig to the patient marker is known. A third option would be to position the registration marker of the first option on the patient body not connected to a clamp. The workflow for calibration may be similar to the first option but the transformation between the registration marker's coordinate system and the patient marker's coordinate system may be computed by optical images, similar to the second option.

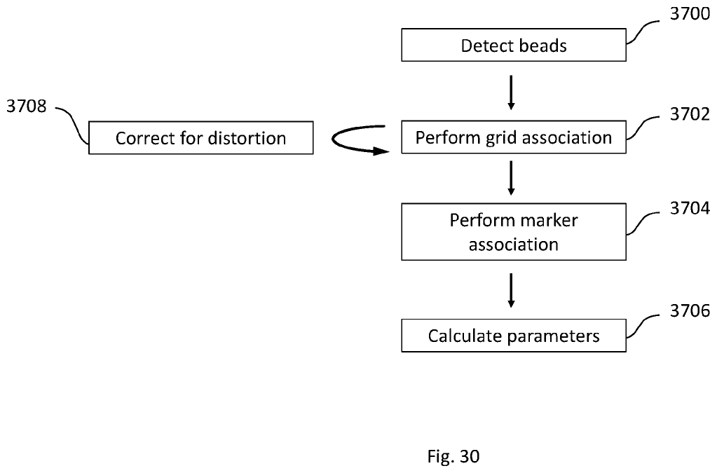

FIG. 30 is a flow chart that schematically illustrates a method for calibration, according to one embodiment. One or more images including the radiopaque beads 40 are first obtained. At step 3700, the beads 40 are detected in the image(s). At step 3702, grid association occurs where the image of the beads 40 disposed at the lower bead plate 408 undergoes a process that results in associating each bead on each line of the bead pattern to correct indices. At step 3704, marker association is performed, where the image of the beads 40 disposed at the upper bead plate 406 undergoes a process that results in associating each bead on each line of the bead pattern to the correct world point. The intrinsic and extrinsic parameters of the fluoroscope 30 (e.g., X-ray detector 34) are calculated at step 3706. Step 3708 illustrates that the upper bead plate (or marker beads) are utilized for distortion correction in the X-ray images as described herein.

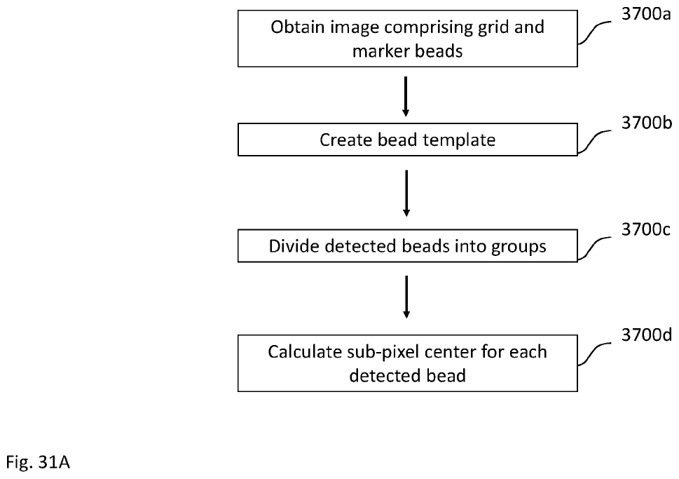

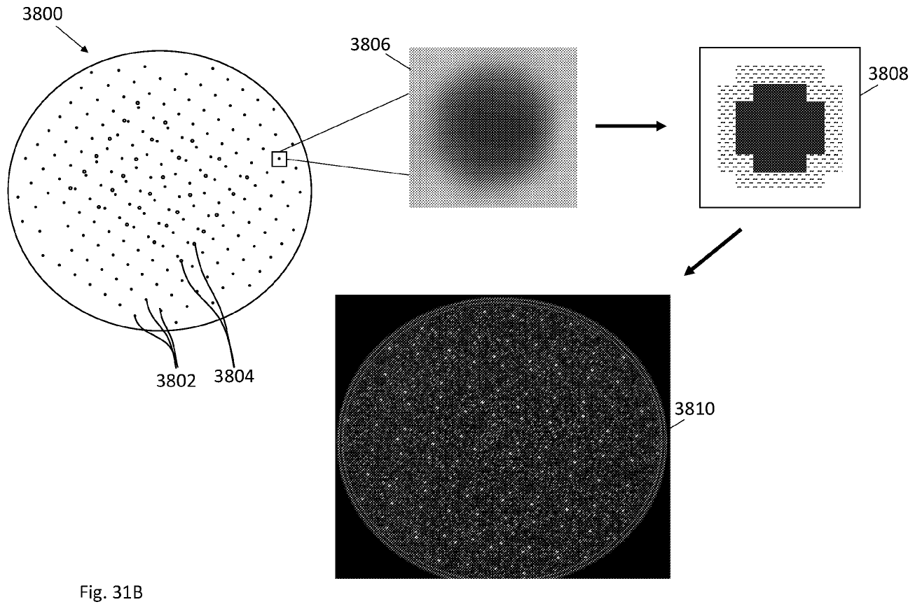

FIGS. 31A-31B are a flow chart and corresponding schematic pictorial illustrations of the steps of the flow chart, according to one embodiment of the disclosure. In FIG. 31A, a process (e.g., subprocess, method or algorithm which may be stored in memory 52 and executed by processor 50) for detecting the beads 40 is outlined. At step 3700a, an image 3800 comprising the grid and marker beads 40 is obtained as illustrated in FIG. 31B. Certain information regarding the bead plates 406, 408 is known prior to capturing images of these bead plates 406, 408. This information includes the expected radius of the beads 40, the spacing between the beads 40, and the number of expected beads 40. Using this information, at step 3700b, a bead template 3808 is created and used to create a correlation image 3810 by moving the bead template 3808 over all the pixels of the original image to find local maxima. For example, when the bead template 3808 is moved over a section of the original image that includes a bead (e.g., bead image 3806 in FIG. 31B), the correlation value will be higher than when the bead template is moved over a section of the original image that does not include a bead. Following this template matching, the detected beads 40 are divided into groups at step 3700c according to size to distinguish the beads on the lower bead plate 408 from the beads on the upper bead plate 406. The sub-pixel centers are then determined for each of the beads at step 3700d.

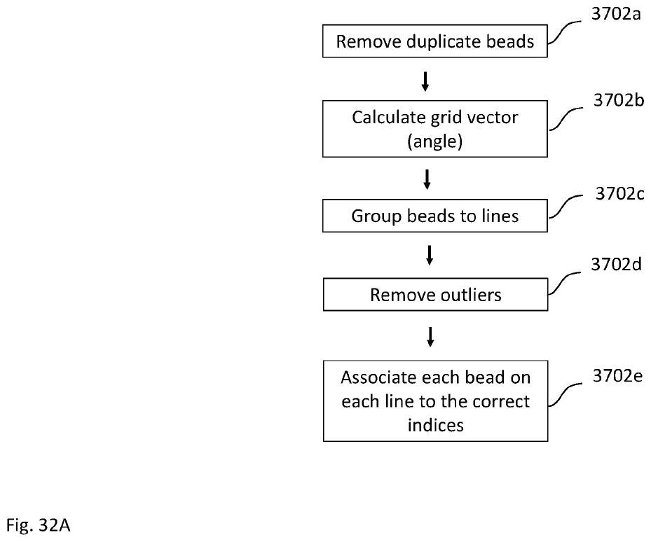

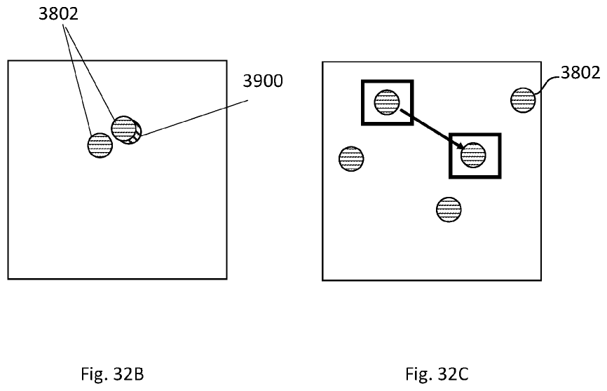

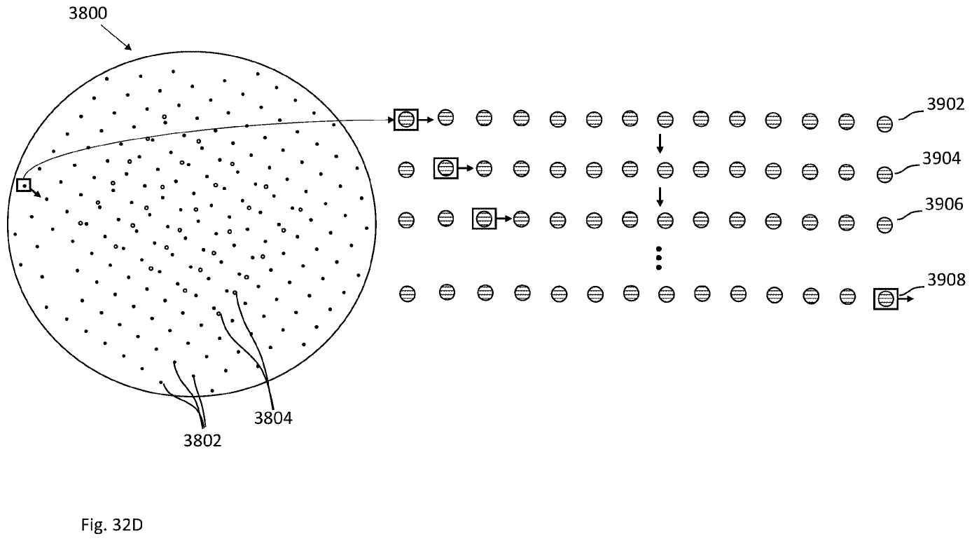

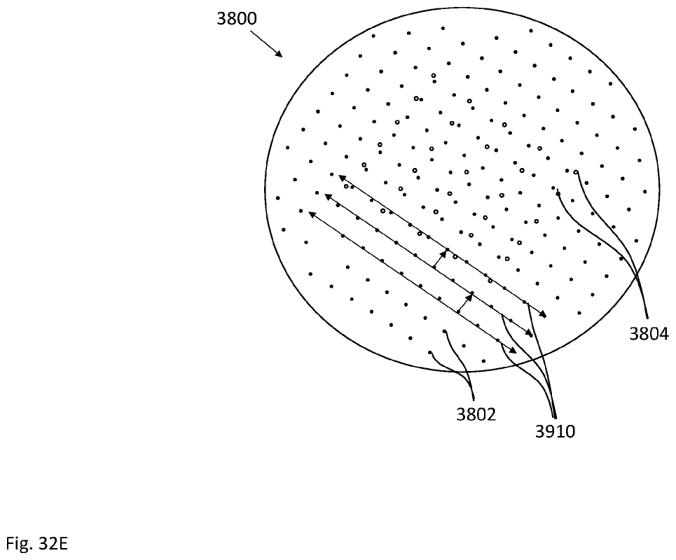

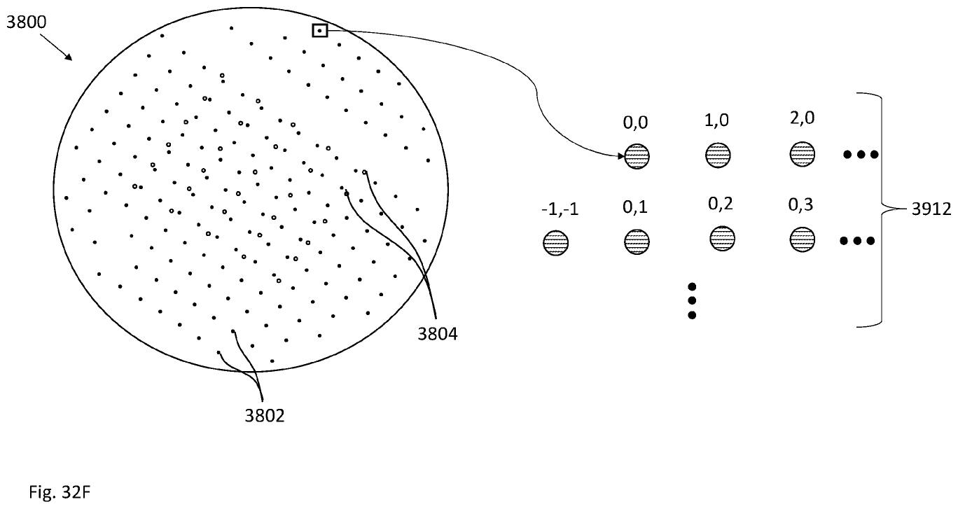

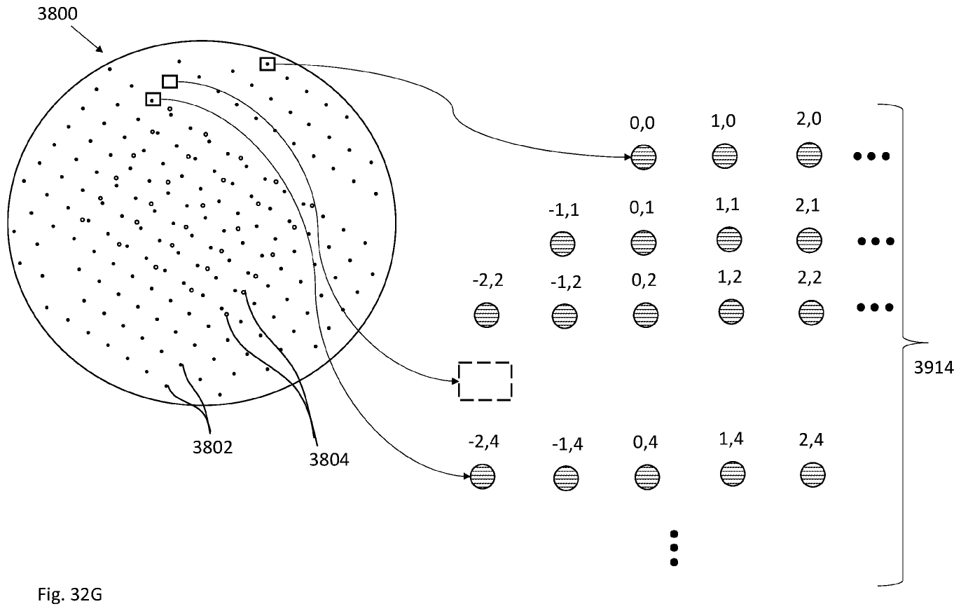

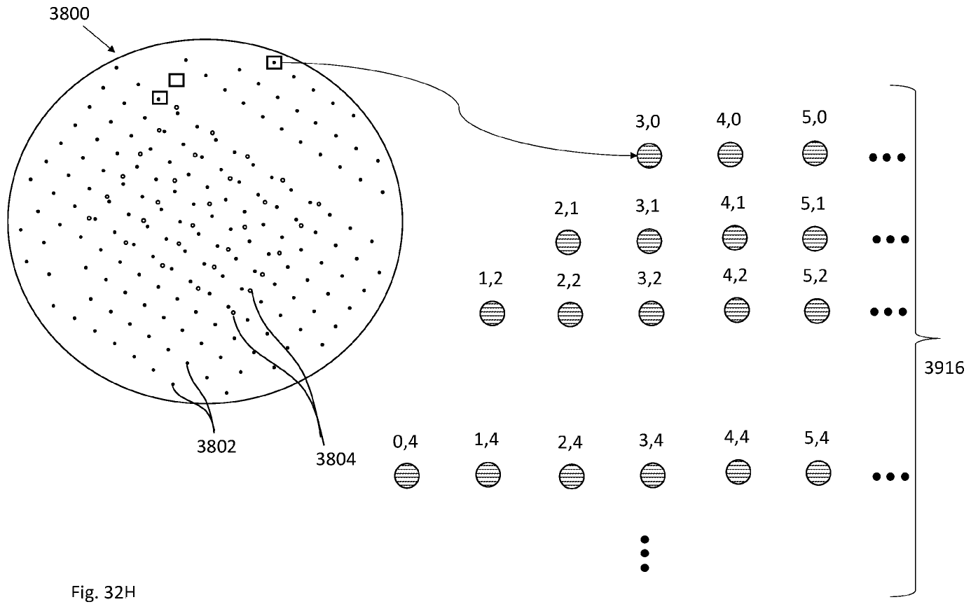

FIGS. 32A-32H are a flow chart and corresponding schematic pictorial illustrations of the steps of the flow chart illustrating grid association, according to one embodiment. Following bead detection, grid association is performed. At step 3702a, duplicate beads are removed. In one embodiment, the duplicate bead removal step of 3702a includes analyzing all the detected beads, and further inspecting beads located within a certain distance of other beads. This step can illuminate if proximate beads are of different types (and thus belong to different bead plates as illustrated in FIG. 32B showing beads 3802 and 3804) or of the same type (and thus possibly a duplicate bead). At step 3702b, a direction vector is calculated for each pair of beads (e.g., beads detected for the lower bead plate 408) separated approximately by the expected grid bead separation distance. This is illustrated in FIG. 32C. The grid vectors are then used at step 3702c to group beads to unique lines. In one embodiment, and as illustrated in FIG. 32D, the grouping can be done by sorting the beads through a process of starting with one grid bead and searching for another bead in the grid direction using the grid vectors (e.g., 3902 shows the identification of the first grid bead, 3904 shows that using the grid vector, the second grid bead is identified along the line, and so forth with the illustrations for 3906 and 3908). Outliers are removed at step 3702d. In one embodiment, and as illustrated in FIG. 32E, distances can be calculated between the determined lines 3910 of beads and used to remove lines of beads based on the expected distances. At step 3702e, each bead on each line is associated with correct indices. As illustrated in FIG. 32F, the indexing can occur through a sub-process 3912 where one bead is selected to have index (0,0) and the rest of the beads are indexed with respect to this initial bead. In some embodiments, if a row of beads appears to be missing where expected based on prior knowledge of the imaged bead plate, then as shown in sub-process 3914 in FIG. 32G, the beads can be indexed to take into account the missing beads. Bead indices may be shifted in sub-process 3916 in FIG. 32H to remove negative indices.

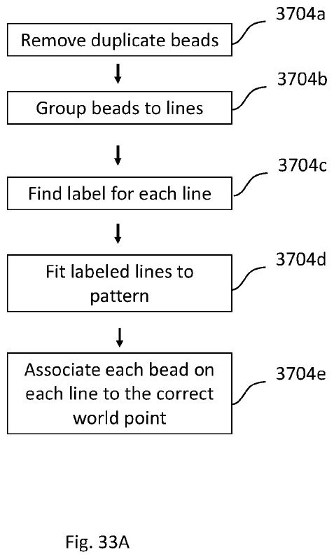

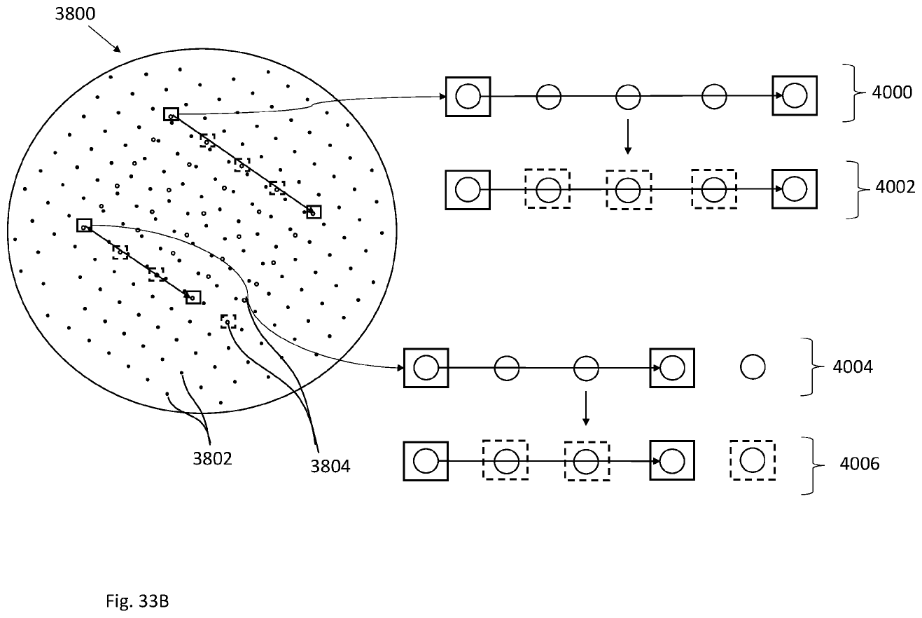

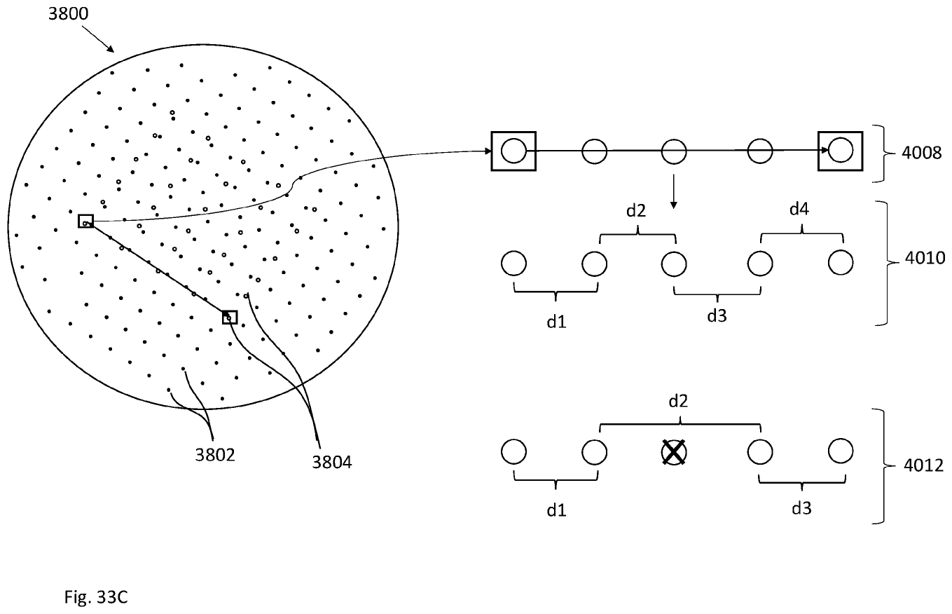

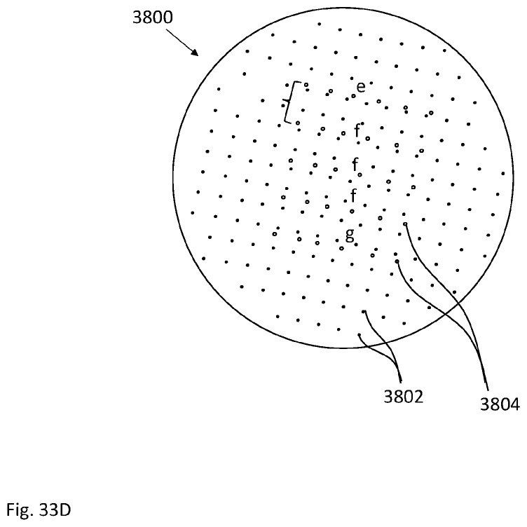

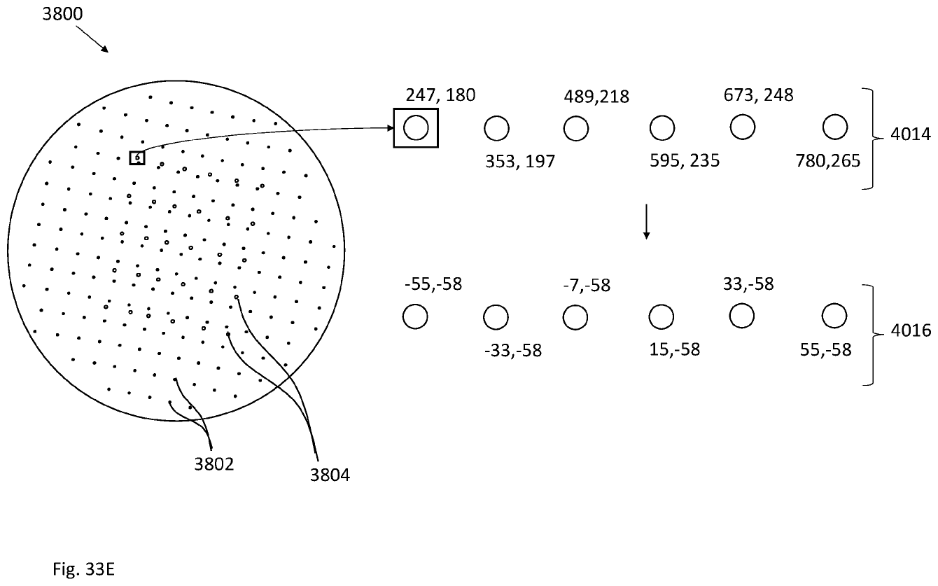

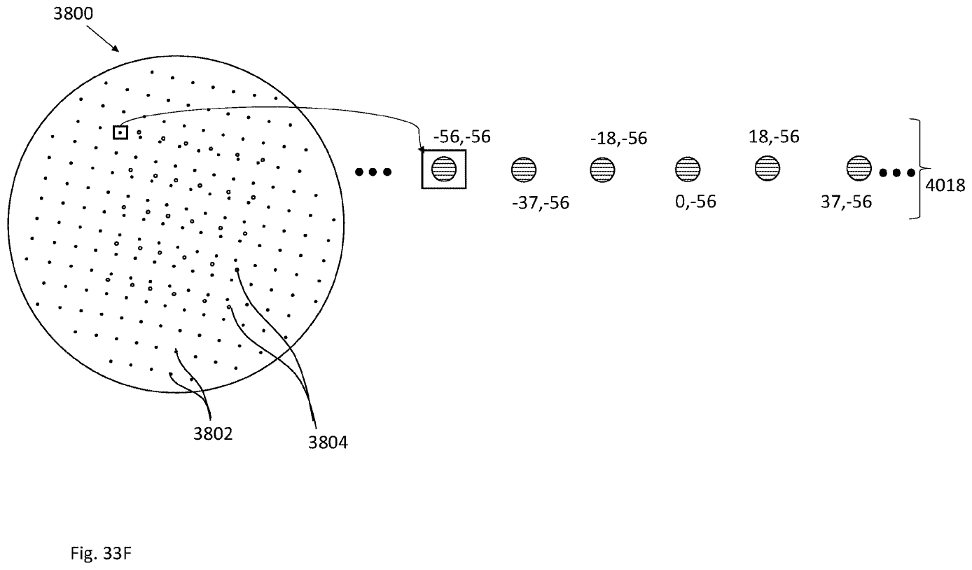

FIGS. 33A-33F illustrate a flow chart and corresponding schematic pictorial illustrations of the steps of the flow chart illustrating marker association, according to one embodiment of the disclosure. Following grid association, marker association is performed. At step 3704a, duplicate beads are removed in the same or similar manner as in step 3702a for grid association. At step 3704b, beads are grouped to unique lines according to grid angle (or the direction vector) as shown, for example, by zoomed-in sub-illustrations 4000, 4002, 4004, 4006 in FIG. 33B. In some embodiments, when a marker is not detected, the grouping of beads to unique lines can accommodate those undetected marker beads as indicated by sub-illustrations 4004 and 4006 in FIG. 33B. Labels are determined for each line at step 3704c based on the spacing of the unique lines and the distances between each pair of beads on each unique line as illustrated in FIG. 33C. An example for one line or grouping of beads is shown in sub-illustrations 4008, 4010, 4012. The labeled lines are fit to a known pattern at step 3704d (FIG. 33D), and each bead on each line is associated to the correct world point in step 3704e. As shown in FIG. 33E, each marker bead or upper plate bead on each line is associated to the correct world point through a transformation from pixels (e.g., bead locations as pixels 4014 in FIG. 33E) to a world point [x,y,0] (e.g., bead locations as world points 4016 in FIG. 33E). Similarly, the lower plate beads 3804 can also be associated to correct world points as shown schematically in FIG. 33F. An example for one line or grouping is shown in sub-illustration 4018.

FIG. 34 is a flow chart that schematically shows details of a method of registering 2D and 3D images, in accordance with an embodiment of the disclosure. While discussed in connection with vertebrae of the spine, the method may also be similarly used for other bones, joints or tissue. To begin the process of registering the 3D image segments with the 2D images of the spine, processor 50 receives an initial input associating one of the vertebrae among the 3D image segments with the locations of the same vertebra in the two 2D images (Block 4100). For example, a user of system 20 may use a cursor to mark the location of a selected 3D vertebra or other bony structure on the 2D images. In addition, in accordance with some implementations, processor 50 makes an initial estimate of the orientation of the spine of patient 24 using external cues (Block 4102). For example, the location of marker 44 or 46 relative to the patient's skeleton indicates the Z-direction (e.g., the sagittal axis), while locations of X-ray source 32 and detector 34 indicate the Y-direction (e.g., the longitudinal axis). Based on the initial input and the estimated orientation of the spine, processor 50 is able to associate each of the 3D image segments with a corresponding vertebra and/or other bony structure in each of the 2D images (Block 4104). The processor 50 may also estimate and make use of the known ranges of movement of the vertebrae and/or other bony structures relative to one another in estimating the registration parameters.

To register the vertebrae in the 3D image segments precisely with the associated vertebrae in the 2D images, processor 50 generates (e.g., calculates) digitally reconstructed radiographs (DRRs) or other simulated radiographic images based on the 3D images of the vertebrae over a range of vertebra movements and rotations around the estimated axes of the 2D images relative to the spine (Block 4106). In some embodiments, the intensity of each pixel in a given DRR is computed by calculating the cumulative radiodensity of the voxels along the path of a ray between the X-ray source and the pixel. In some implementations, the DRR(s) may be generated using Siddon's algorithm.

In the illustrated embodiment, processor 50 applies a process of optimization (Block 4108) to find the orientation of each 3D vertebra or other bony structure relative to the 2D images, by comparing the gradients of the pixel values in the DRR P₁(i, j) to the actual gradients of the pixel values in the 2D X-ray images P₂(i, j). The optimization uses mask functions M₁(i, j), M₂(i, j) for the DRR and X-ray images, respectively, to mitigate the effect of artifacts, such as foreign objects in the X-ray image. (For example, the mask value may be set to zero for pixels containing beads of the calibration jig 38.) For purposes of the optimization, processor 50 calculates a similarity measure, or metric, Sim(P₁, P₂) between each DRR and the corresponding X-ray image, using the following formulas, for example:

$$α ( i , j ) = {cos}^{- 1} ( max ( \frac{∇ {P}^{1} ( i , j ) * ∇ {P}^{2} ( i , j )}{\vert ∇ {P}^{1} ( i , j ) \vert * \vert ∇ {P}^{2} ( i , j ) \vert} , 0 ) )$$

$$w ( i , j ) = {M}_{1} ( i , j ) * {M}_{2} ( i , j ) * cos ( 2 * α ( i , j ) )$$

$$Sim ( {P}_{1} , {P}_{2} ) = {\sum }_{i , j} \{ \begin{matrix}w ( i , j ) * min ( \vert ∇ {P}^{1} ( i , j ) \vert , \vert ∇ {P}^{2} ( i , j ) \vert ) , & w ( i , j ) > 0 \\ w ( i , j ) * \vert ∇ {P}^{1} ( i , j ) \vert , & w ( i , j ) < 0\end{matrix}$$

The similarity is averaged over all pairs of (X-ray image, DRR). The optimal orientation and location belong to the pair with the highest average similarity measure, or metric.

Various search strategies can be used to find the optimal orientation and location, while avoiding the excessive computational burden of an exhaustive search. For example, the search space of orientations and locations may be divided into smaller regions, and the similarity measure may be computed for one sample in each region. Processor 50 may then perform a fine-grained search only within the regions that had the highest similarity measures. The search may be performed initially at coarse CT resolution (for example, 1 mm, 1.5 mm. 0.5 mm, 2 mm, or other values) and then refined using a finer CT resolution (for example, 0.3 mm, 0.2 mm, 0.15 mm, 0.1 mm 0.05 mm, or other values). In some implementations, the search space may be sampled to avoid optimization getting stuck in local minima. After the first vertebra is registered, its neighbors may be registered using the registration of the first vertebra as the initial guess. Other bony structures may also be similarly registered.

Once the optimal locations and orientations of all the vertebrae or other bony structures in the 3D image segments have been found in this manner, processor 50 uses the results in reconstructing a complete 3D model of the spine from the individual 3D vertebrae and/or other bony structures (Block 4110). In accordance with several embodiments, the locations and orientations of the vertebrae and/or other bony structures in this 3D model will match the actual spine (e.g., the actual pose of the spine) of patient 24 on operating table 26. At Block 4112, processor 50 may then display the 3D model (e.g., generate an output for display to facilitate navigation of medical tools in the procedure). When AR is utilized, processor 50 may then use the relative location and orientation of head-mounted AR display unit 28 or head-mounted AR display unit 70 with respect to patient 24 to calculate the views of the vertebrae and/or other bony structures that will be projected onto displays 60 in the proper locations and orientations, overlaid on the actual anatomy of patient 24.

FIG. 35 is a schematic representation of a segmented 3D image of a vertebra, in accordance with an embodiment of the disclosure. To arrive at this image, the 3D image of the spine in a CT scan is segmented into individual 3D vertebrae. This segmentation operation can advantageously be carried out by deep learning techniques, using one or more trained convolutional neural networks (CNNs). The sacrum and ilium may be segmented in this manner, as well, using a separate neural network from the neural network used for the vertebrae or the same neural network.

In some embodiments, a combination of three networks is used for this purpose. The networks are fully convolutional networks (e.g., based on the U-Net architecture).

A first network may receive as input a CT image or other tomographic or volumetric of the spine or of a portion of the spine resampled to a coarse resolution with respect to the resolution of the CT image (e.g., the original, non-processed CT image). For example, the CT image may be resampled to a resolution of 8 mm per voxel (e.g., 8*8*8 mm³). According to some embodiments, the coarse resolution may be in the range of 5-15 mm. According to some embodiments, the coarse resolution may be in the range of 6-10 mm. In some instances, a CT image resolution may be down to 2 mm. Such coarse resolution may allow feeding the entire image to the network (e.g., as one block) and saving in computing resources. The output of the first network may be two values for each voxel: one a value indicating if a vertebra portion is included in the voxel; the second a value indicating if a portion of the sacrum or ilium is included in the voxel. The aim is to define an area of interest in the image. In accordance with several implementations, processing of an image of a smaller size advantageously allows the use of less computing resources and a faster processing. Furthermore, identifying the area of interest may prevent errors, such as identifying other bone structures adjacent to the spine (e.g., the shoulder) as the area of interest (e.g., as the spine).

A second network may receive as input the CT image area identified by the first network as the area of the sacrum and ilium resampled to a finer resolution with respect to the resolution used in the first network (e.g., of 1 mm). In some embodiments, the fine resolution may be between 0.3 and 1.5 mm. In some embodiments the fine resolution is finer with respect to the CT image resolution. In some embodiments, the fine resolution is substantially equal to the CT image resolution. In some embodiments, the fine resolution may be equal to or between 50% less than CT image resolution and 50% more than CT image resolution. The resampled relevant image portion may be then divided into patches of a predefined voxels size. The network is fed with one patch at a time. The output may include two values per voxel: a value indicating if a portion of the sacrum is included in the voxel and a value indicating if a portion of the ilium is included in the voxel. In accordance with some implementations, the aim is to segment the sacrum and ilium.

A third network may be then applied to the area of interest in the CT image identified as including vertebrae by the first network. The third network may receive two inputs: a patch of the portion of interest of the CT image resampled to a fine resolution (e.g., of 1 mm). This fine resolution may be equal or substantially equal to the fine resolution used in the application of the second network; the same patch including information with respect to the previously segmented vertebra. In some embodiments, the first such patch may include information with respect to the segmented sacrum and ilium. The output of the network may be a patch including a value for each voxel indicating if the voxel includes a portion of the vertebra following the previously segmented vertebra (or ilium and sacrum at the beginning) identified in the input patch (the second input above). That is to say, the output is the segmentation of the next, adjacent (in a predefined direction) vertebra. In some implementations, the network is trained to identify the next vertebra in the first patch based on the second patch which includes information identifying the previously segmented vertebra, and to change the location of the patch in the resampled CT image along a predefined spine direction (down-up or vice versa) until the entire next vertebra is identified and centralized in the patch. In the present example, down-up direction is used, beginning with the ilium and sacrum.

The direction of the CT scan (e.g., patient body orientation: legs-head) may be determined by the DICOM standard data provided with the scan. Alternatively, it may be determined by identification of the sacrum and/or ilium. The operation of the third network may end when at least one of the following occurs: the entire area of interest has been processed, or 28 vertebras are identified. If the sacrum or ilium are not identified in the CT image by the first network (e.g., when the CT scan does not include the sacrum and ilium), then the second network may not be applied. The outputs of the networks may be transformed to binary values (e.g., “0” and “1”), and a mask image of the CT image may be generated based on these values and correspondingly indicating the segmented vertebrae and ilium and sacrum, if included in the CT image. In accordance with several embodiments, the training of the networks is supervised. Thus, spine images are labeled for each network training, serving as the ground truth. In some embodiments, augmentation may be used (e.g., by application of transformations to the training images to increase the number of different training images). According to some embodiments, the training CT images used in training of the first network includes labeling of voxels in the spaces between the vertebrae as vertebrae (“smearing” of the vertebrae) to facilitate the vertebra area identification and segmentation.

In accordance with several embodiments, processor 50 divides the segmented spine into 3D image segments, each containing a single vertebra (e.g., according to the method described above). This segmentation step calculates and crops a 3D bounding box around each vertebra, as illustrated by the three views shown in FIG. 35. Any soft tissue and parts of neighboring vertebrae that remain in the bounding box are deleted. All voxels in the image segment that do not belong to the bone of the vertebra are assumed to belong to soft tissue and are set to 0 HU (Hounsfield Units). The resulting isolated 3D images of the vertebrae will be used subsequently in registration of the 3D and 2D images. The segmentation output may be a vector of small CT image portions (e.g., one for each vertebra, sacrum and ilium).

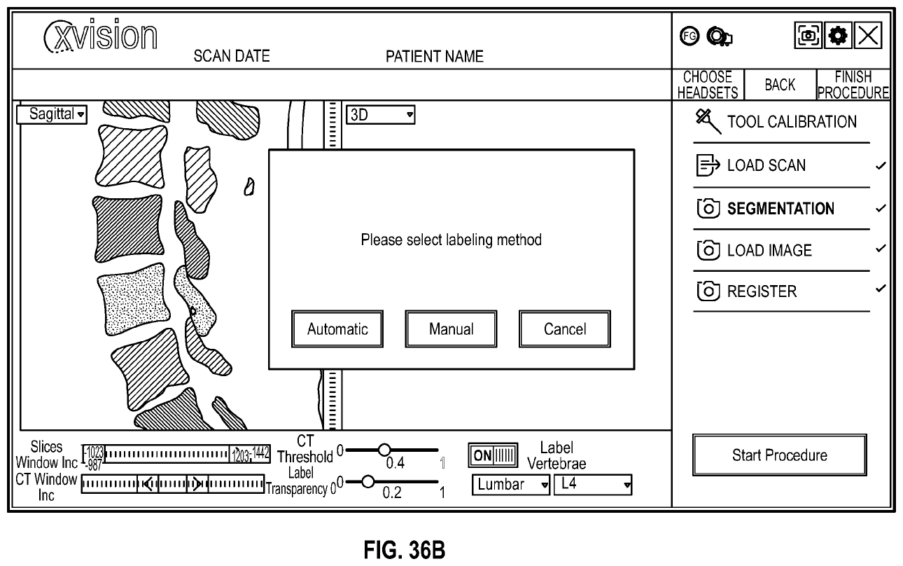

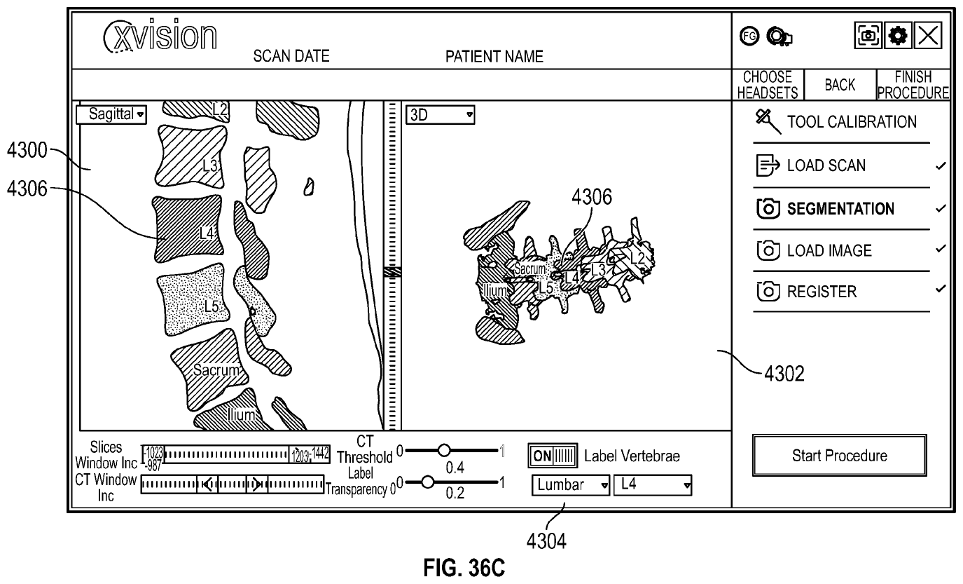

FIGS. 36A-36C and FIGS. 38A-40B show screen shots of an example implementation of a GUI and user workflow for registering a CT image of a patient's spine and two fluoroscopic images of the patient's spine captured from two different angles (e.g., anteroposterior (AP) and lateral or oblique-lateral). The concepts described can be implemented for images of other bones, joints, or other tissue (e.g., other non-spinal orthopedic locations, cranium, ear-nose-throat, mouth, shoulder, hip, knee, arms, feet, ankles).

Reference is now made to FIGS. 36A-36C, which show screen shots of a segmentation display of the GUI. As disclosed hereinabove, the CT image may be automatically segmented. The automatically segmented CT image may be then displayed to the user in a segmentation display (e.g., via views or images 4300 and 4302 of the segmented image). The automatically segmented CT image may be displayed in various views, including CT slice views, such as sagittal view 4300 or coronal view, 3D views such as view 4302 and/or x-ray or “X-ray-like” views. In some embodiments, the CT slice views relate to the different anatomical planes (e.g., coronal, sagittal and axial). Sagittal view 4300 shows the entire spine segmentation; however, in some embodiments, portions of the spine are segmented. Slides 4310 or other GUI input elements may allow the user to slide, toggle, or otherwise transition between the different slices or segments and the slices or segments may be activated once a slice view is displayed. A GUI input element 4304 may allow a user to toggle between various portions or segments of the vertebrae or other bony segments. In some embodiments, one or more GUI elements may be included to allow the user to adjust the visualization of the CT slices, such as slider 4330 (e.g., window center and window level). 3D view 4302 may display a 3D model or 3D rendering of the spine 4306 generated from the CT scan. In some embodiments, the 3D model or rendering of the 3D view may be manipulated by the user (e.g., rotated in various or all directions). The x-ray or “x-ray-like” views may be generated from the CT scan (e.g., via DRRs). In some embodiments, the x-ray views or virtual x-ray views may be generated to substantially match or resemble the point of view of the captured fluoroscopic images. In some embodiments, the virtual x-ray views are at a predefined fixed view (e.g., AP and lateral or oblique-lateral). In some embodiments, one or more GUI elements may be generated or provided to allow the user to adjust the threshold based on which the DRR image is generated and/or to allow the user to adjust the visualization of the DRR image display (e.g., via adjustment of pixel intensity or opacity values). In some embodiments, the user may select the view to be displayed (e.g., via a drop-down menu 4305). The segmentation GUI display may include one or more windows to display one or more views simultaneously, optionally, according to user selection and as shown in FIGS. 36A and 36C, which include two view windows. In some embodiments, the spine segmentation may be further visualized by coloring the different segmented vertebrae in different colors. The spine segmentation may additionally or alternatively be visualized by different shading or hatching patterns. In some embodiments, and as illustrated in FIGS. 36A-36B, the different segmented vertebrae may be automatically labeled (e.g., by activating GUI element 4340), which may be a toggle switch or other GUI element. In some embodiments, the segmented CT image may be manually segmented or both manual and automatic segmentation may be allowed or performed. In some embodiments, a GUI element may be generated (e.g., slider 4345) to allow the user to control the label transparency (e.g., to allow display of labels without concealing essential image information). In some embodiments, manual edit of the automated segmentation may be allowed (e.g., by activating GUI element 4350 (e.g., toggle switch)). At the end of the segmentation phase of the CT-Fluoro image registration, the registration procedure, phase or step may be initiated (e.g., by pressing the command button “Start Procedure” or other GUI element).

FIG. 37 is a schematic pictorial illustration of the 2D/3D registration process, in accordance with an embodiment of the disclosure. For the sake of illustration, this figure shows a portion of a spine, but the same principles are applied in registration of each of the vertebrae or other bones. “Image 1” and “image 2” represent 2D fluoroscopic views of the bone captured with X-ray detector 34 at two different angles (viewpoint 1 and viewpoint 2), so that each 2D image represents a projection of the 3D shape of the bone onto a different plane. To find the actual location and orientation of the bone (be it a femur or a vertebra or other bone), processor 50 is configured to calculate coordinate transformations (TCT) 1 and (TCT) 2. The coordination transformations may be calculated prior to registration.

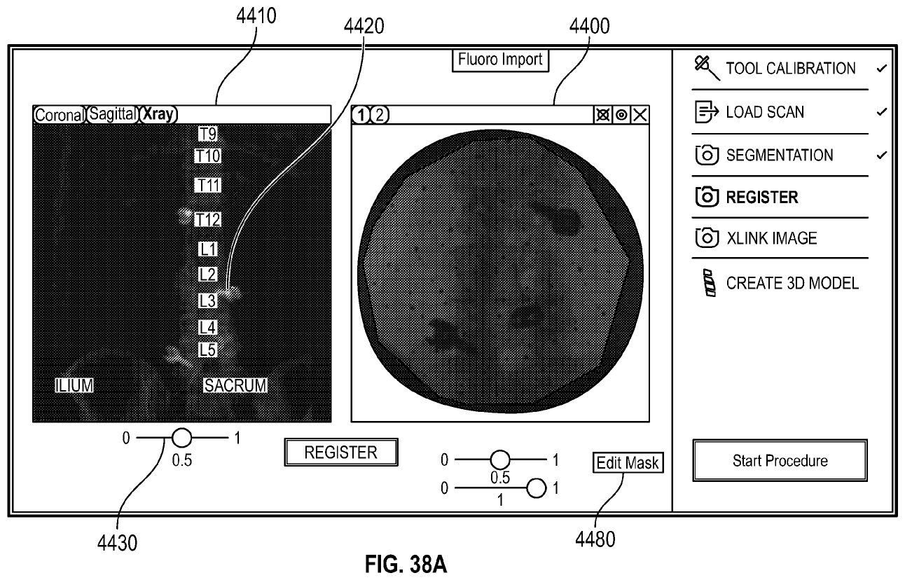

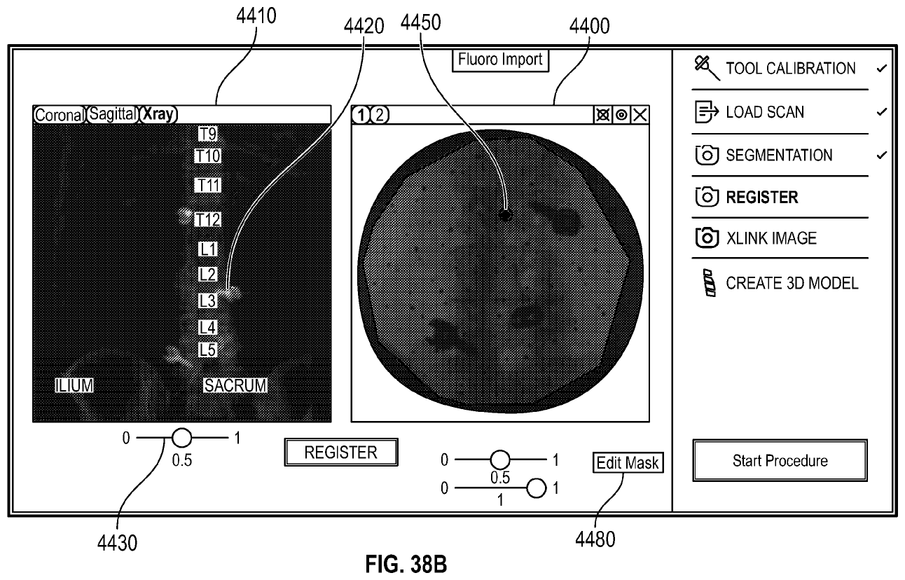

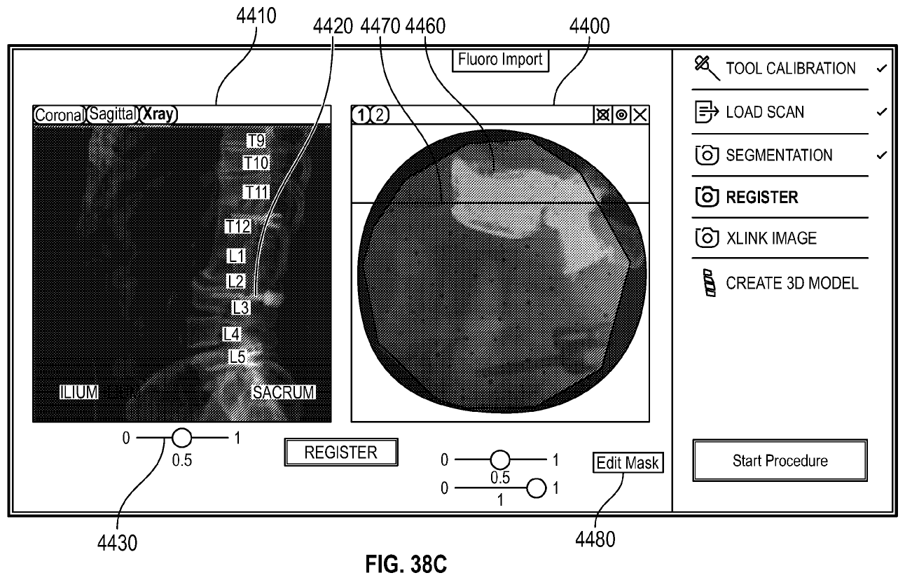

FIGS. 38A-38C are screen shots of an example implementation of a GUI display for registering the fluoroscopic images with the segmented CT image. The segmentation display includes Fluoro view window 4400 and CT view window 4410. In Fluoro view window 4400, the captured fluoroscopic images may be displayed, and specifically, the first and the second fluoroscopic images may be displayed in two views indicated “1” and “2”, respectively. In some embodiments, CT window 4410 displays a virtual x-ray image from a point of view or at a view angle corresponding to the point of view or view angle from which the currently displayed fluoroscopic image was captured. In FIGS. 38A-38B, the first fluoroscopic image is displayed in window 4400 and a first corresponding virtual x-ray image is displayed in window 4410. In FIG. 38C, the second fluoroscopic image is displayed in window 4400 and a second corresponding virtual x-ray image is displayed in window 4410. In some embodiments, window 4410 may include additional CT views, such as sagittal and coronal slice views, while the user may switch between the different views, as shown in FIGS. 38A-38C. In some embodiments, the virtual x-ray image segmented portions (e.g., vertebra) may be labeled. The user may then input an initial guess or indicate matching vertebrae to initiate the automatic segmentation. The user may indicate a segmented vertebra in the virtual x-ray image (or any other CT-based image) (e.g., via blue highlighting or other coloring 4420 of vertebra L3). The indication may be performed with a user input device (e.g., keyboard, mouse, control pad, joystick, touchscreen user interface, and/or the like). A mark, such as target 4450, may be then located by the user on the matching vertebra in one of the fluoroscopic images, as shown in FIG. 38B. The second fluoroscopic image may be then displayed with a mark (e.g., line 4470, which may be an epipolar line calculated from the mark on the first fluoroscopic image), which indicates where the selected vertebra (e.g., L3) is located in the second fluoroscopic image and as shown in FIG. 38C. The user may then locate another mark or indication, such as blue highlighting or coloring 4460 in the shape of the selected vertebra, along line 4470 (e.g., an epipolar line) to mark the corresponding vertebra (e.g., select vertebra position) in the fluoroscopic images, as shown in FIG. 38C. In some embodiments, the registration display may include a GUI element, such as slider 4430, to allow the user to adjust the threshold used in generating the virtual x-ray image. In some embodiments, GUI elements which allow the user to adjust the image display or visualization (e.g., window center and window level) may be included. The user may optionally interact with the user interface to rotate the vertebrae for a better match. In some embodiments, the user may be allowed to mask the fluoroscopic image (e.g., by activating GUI element 4480). For example, the user may mask portions of the image including noise or data which may interfere with the registration process. Thus, the user may mask metal elements, such as screws, as shown, for example, in the fluoroscopic images displayed in window 4400.

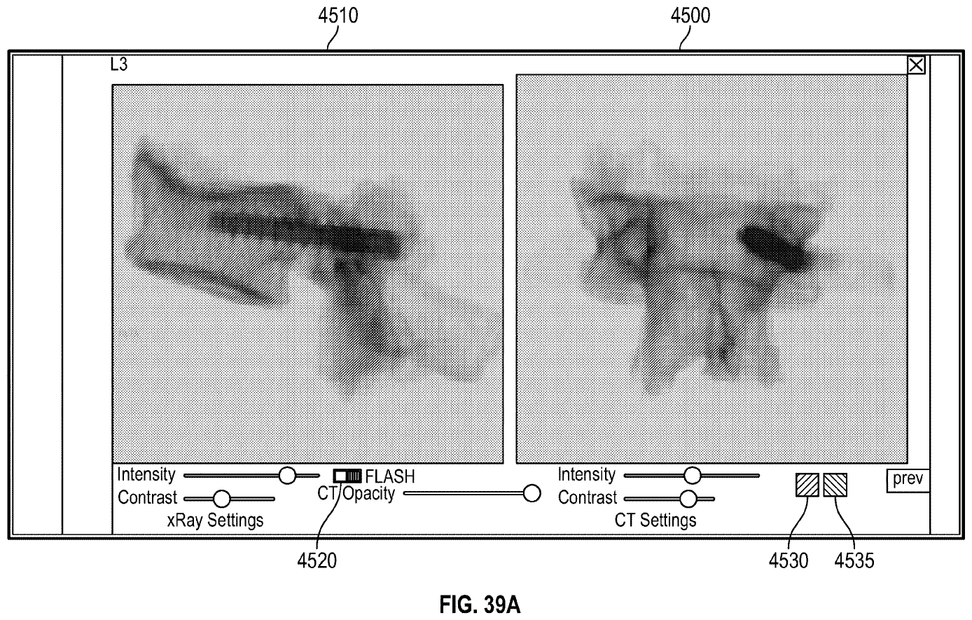

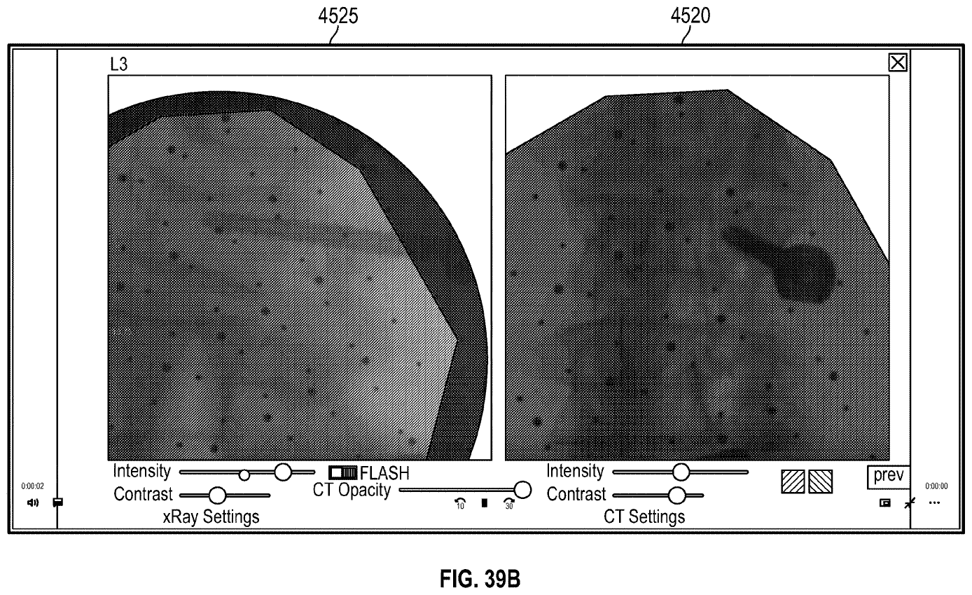

Reference is now made to FIGS. 39A-39B, which are screen shots of the GUI display showing different views of the registered vertebra (L3) in the segmented CT image overlaid on the Fluoro image (or vice versa). FIGS. 39A-39B display an augmented image or a combined image of the registered vertebra comprised of a fluoroscopic image of the registered vertebra and a corresponding virtual x-ray image, generated assuming the point of view of the fluoroscopic image. In accordance with several embodiments, such a combined image may visualize the registration and advantageously allow a relatively straight forward evaluation, or at least assist in the evaluation of the registration. The x-ray virtual image may be blended with the registered segmented vertebrae. In several implementations, each of the registered segmented vertebrae may be placed and rotated according to the registration output, meaning that the relations of the vertebrae positions and orientations are not necessary as on the CT image. In some embodiments, a GUI element (e.g., slide element) may be included which allows the user to adjust CT transparency or opacity, either continuously or in discrete increments. In some embodiments, when adjusting the CT transparency to the minimum value, the combined images will only or mostly display the x-ray virtual image, as shown in views 4500 and 4510. In some embodiments, when adjusting the CT transparency to the maximum value, the combined images will only or mostly display the fluoroscopic image, as shown in views 4520 and 4525. Intensity and contrast levels of the images may also be adjusted by interaction with GUI user input elements, such as slide bars or adjustment elements. In some embodiments, the user may select a display in flash mode (e.g., via GUI element 4520, which may be a toggle button or switch), in which the images of FIG. 39A and FIG. 39B (e.g., of the virtual x-ray and fluoroscopic image) are sequentially and repeatedly presented in a flashing manner (e.g., while each type of images is presented for a very short, predefined time interval). Such a display may facilitate review of the registration. In some embodiments, the user may be required to approve each vertebra registration. As shown in FIGS. 39A-39B, in some embodiments, the user may press a green button 4530 or other GUI user input element to confirm the vertebra registration and a red button 4535 or other different GUI user input element to reject a vertebra registration. Elements such as CT transparency adjustment and flash display mode may facilitate the registration confirmation process. In some embodiments, a landmark may be added to the combined or augmented image (e.g., visualized such that it only or mostly displays the fluoroscopic image or the virtual x-ray image) by the user. The user may then change the image visualization to receive a second different view of the combined or augmented image (e.g., by adjusting CT transparency level of the image to receive the virtual x-ray image or the fluoroscopic image, respectively) and review the landmark in the second view of the image or in flash mode. The landmark may indicate, for example, an anatomical structure which is discerned and/or of interest. The landmark may facilitate the registration confirmation process.

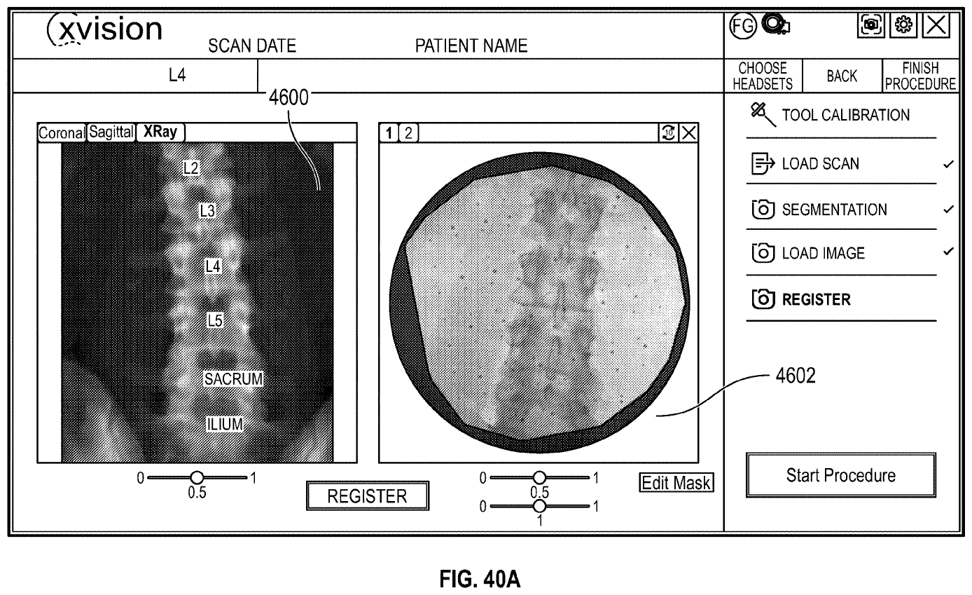

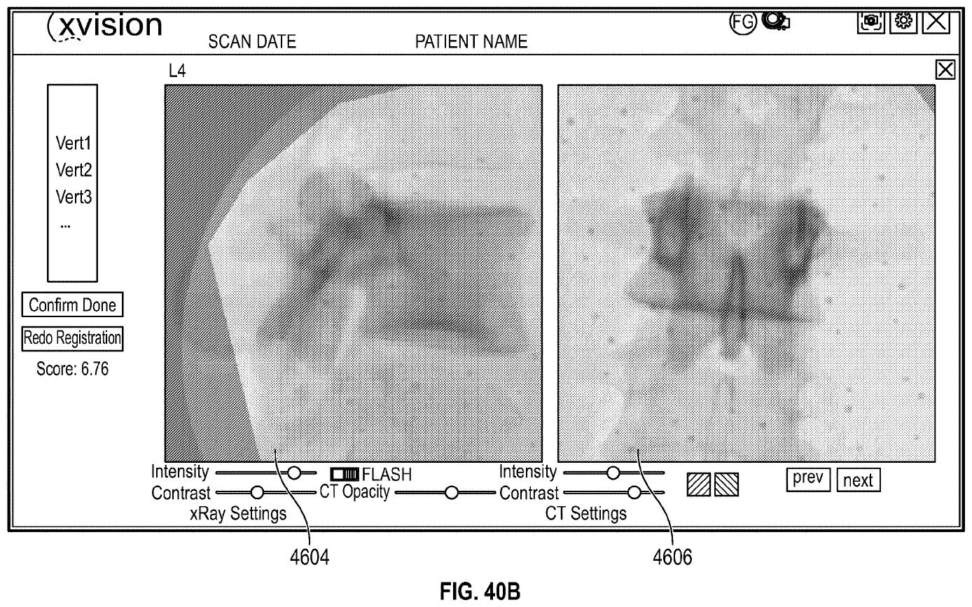

FIGS. 40A and 40B provide additional examples of a GUI display for registering fluoroscopic images with the segmented CT image, with FIG. 40B illustrating different views 4604, 4606 of registered vertebra L4 in the segmented CT image overlaid on the fluoroscopic image. The GUI display of FIGS. 40A and 40B may include similar operational features and elements as the GUI displays described previously.

### Registration of Magnetic Resonance and Fluoroscopic Images

MR images contain a wealth of data regarding soft tissue, such as locations of muscles and nerves, which can be valuable to the surgeon in performing image-guided surgery. Conventional MR images, however, do not show bones clearly (in contrast, for example, to X-ray based CT images). It can therefore be difficult to position the soft tissue in the MR images with sufficient precision in an AR display to enable the surgeon to visualize both the bones and soft tissue together.

In addition to pre-operative ore pre-acquired CT images, embodiments of the disclosure that are described herein provide methods, systems and computer software products that can be used to register a pre-acquired MR image with actual bones in a target region of the patient's body and to fuse the MR image data, including soft tissue segments, with 3D images of bone segments on a display. The calibration and registration techniques described herein in connection with CT may be similarly applied to MR image data (e.g., with the similarity measure, or metric, being different and the training of segmentation neural networks potentially being different). In these embodiments, a 3D MR image of a target region, which includes one or more bones on which surgery is to be performed, is processed to produce a segmented 3D image comprising both bone segments and soft tissue in proximity to the bone segments. This segmented 3D image is registered with the body of the patient by aligning the bone segments in the segmented 3D image with one or more bones in the target region of the body. The registered segmented 3D image is presented on a display, for example in an AR image containing the bone segments and soft tissue overlaid on the target region of the body.

The embodiments that are described below relate specifically to registration and presentation of images of the vertebrae for purposes of spinal surgery; but the principles of the present embodiments may be applied, mutatis mutandis, to other bones and target regions in the body, such as shoulders, hips, knees, arms, legs, head, brain, skull, jaw, ankles, feet, or target regions including organs. Furthermore, the principles of the present disclosure may be applied, mutatis mutandis, in soft tissue surgery.

In some embodiments, the MR image is processed to identify and segment both the bone segments and the soft tissue in the MR image. In accordance with several embodiments, this approach is advantageous since when the same MR image is segmented to identify both bone segments and soft tissue, the segments of bone and soft tissue are inherently registered with one another. Any suitable methods for MR image acquisition and processing may be used for this purpose. One method that may be used, for example, is described in U.S. Pat. No. 10,748,309, whose disclosure is incorporated herein by reference. (Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.) As another alternative, image processing methods, for example using artificial intelligence, such as methods based on neural networks, may be applied to segment the MR image.

Alternatively, or additionally, a CT image may be received and segmented to identify the bone segments, while the MR image is segmented to identify the soft tissue. The MR and CT images may then be registered with one another to produce a segmented 3D image containing both bone segments and soft tissue. The registration of MR and CT images may be performed, for example, by identifying and aligning landmarks in the two images, such as anatomical landmarks or artificial landmarks attached to the patient's body.

In some embodiments, 2D fluoroscopic images of the target region are used in registering the MR image data with the patient's body. For this purpose, two or more 2D fluoroscopic images are captured of the target region of the patient's body. The frame of reference of the 2D fluoroscopic images is calibrated (as described herein) relative to the body, and the bone segments in the segmented 3D image are registered with the bones appearing in the 2D fluoroscopic images, for example using digitally reconstructed radiograms (DRRs).

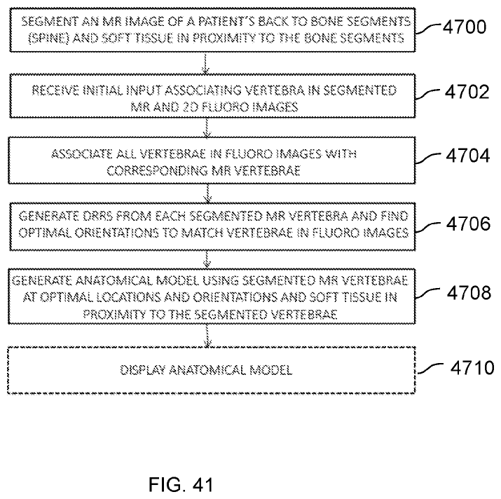

In addition to receiving 2D X-ray images from fluoroscope 30 in FIG. 1, processor 50 is configured to receive 3D medical images of patient 24, including MR images and possibly also CT images. These 3D images are typically acquired prior to the surgery and are stored in a memory 52. Alternatively or additionally, according to some embodiments, the CT image may be generated intraoperatively, in which case there is no need for a fluoroscope 30 and for the 2D X-ray images. Although pre-operative images are typically described herein as 3D images, 2D, 4D or other pre-operative images may also be used. In some embodiments, processor 50 is configured to segment the 3D images to identify bone segments and/or soft tissue, and to register the 3D bone segments with respective vertebrae in the 2D fluoroscopic images. Processor 50 may be further configured to present an image of the spine comprising the registered 3D bone segments and optionally soft tissue on head-mounted AR display unit 28, such that the vertebrae in the 3D images are aligned with the actual vertebrae of the patient's spine. Details of this process are described with reference to FIG. 41 below.

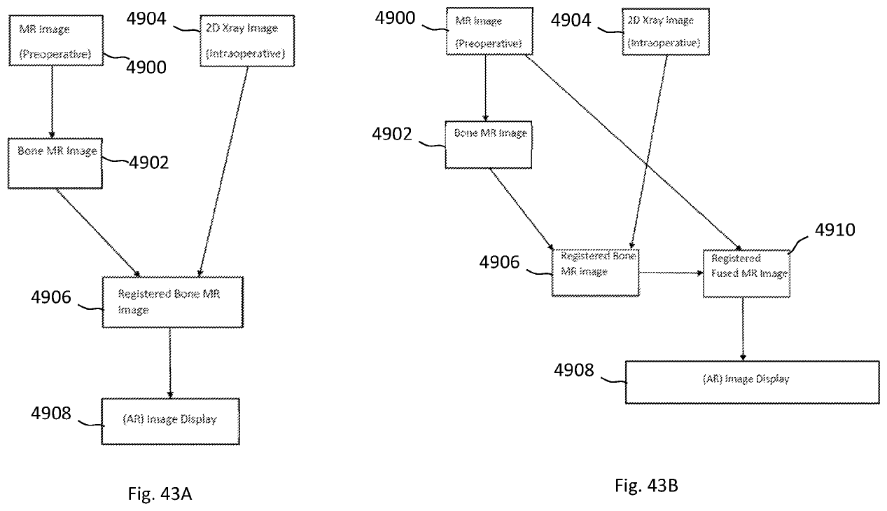

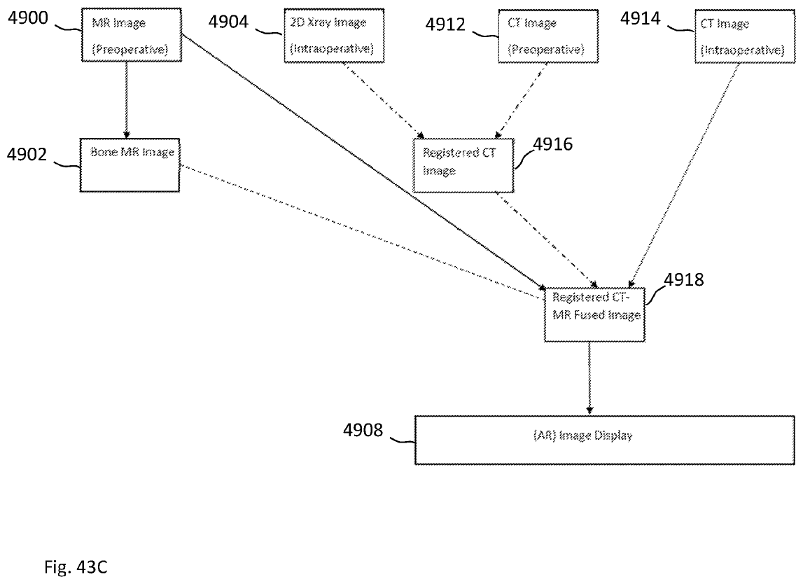

Alternatively, the registered 3D bone segments and soft tissue may be presented on a different sort of display, for example on an AR display that is mounted on patient 24 or on operating table 26 above the surgical site or another local display and/or on a remote display device. Additionally, or alternatively, the registered 3D segments may be presented on a non-AR display, such as a display of a workstation or of a hand-held computer. Other techniques for image registration and/or image fusion are described hereinbelow with reference to FIGS. 43A, 43B, and 43C.

In accordance with several embodiments, processor 50 comprises a general-purpose computer processor, which is programmed in software to carry out the functions of segmentation, calibration, registration, and display that are described herein. This software (e.g., executable program instructions) may be stored on tangible, non-transitory computer-readable media, such as optical, magnetic, or electronic memory media. The software may be stored in memory 52. Additionally or alternatively, at least some of the functions of processor 50 may be carried out using special-purpose computing hardware, such as a graphics processing unit (GPU). Processor 50 may include one or more processors. Processor 50 may be located in a workstation and/or in head-mounted AR display unit 28 (FIG. 2A) or may be located remotely (for example in a cloud platform on one or more remote servers).

In one embodiment, the AR image includes one or more vertebrae or other bony structures that are segmented from a 3D medical image (MR or CT, for example), as well as soft tissue in proximity to the vertebrae segmented from an MR image or a CT image. This AR image is projected onto an overlay area 62 of displays 60 (FIG. 2A) in alignment with the anatomy of the body of patient 24, which is visible to surgeon 22 through displays 60. Overlay area 62 may be transparent, semi-transparent or opaque. In other words, the images of the vertebrae are overlaid on the actual locations of the corresponding vertebrae in the spine of patient 24 (either on top of the skin for minimally invasive surgery or overlaid on the actual vertebrae for open surgery).

To align the AR image with the patient's anatomy, one or more cameras 48 (e.g., infrared cameras or other optical cameras) capture respective images of a field of view (FOV), which may include, for example, marker 44, marker 42 and marker 44, or marker 44 and registration marker 46. Processor 50 processes the images of one or more of markers 42, 44, 46, for example, to register marker 44 with the patient's body and to determine the location and orientation of display unit 28 with respect to the patient's body. Based on this registration and the determination of the current location of display unit 28, processor 50 is able to select the appropriate vertebra or portion of the spine to display in the AR image in overlay area 62 and to set the appropriate magnification, translation, and orientation of the vertebrae and soft tissue in the AR image to match the underlying structure of the patient's spine as seen from the point of view of surgeon 22. The one or more cameras 48 may be used to optically track the location of patient 24, for example via marker 44. The one or more cameras 48 may include two cameras as shown in FIG. 2A (e.g., a left camera and a right camera) or two additional cameras to provide a stereoscopic display of at least a portion of the field of view of the surgeon as captured by the two cameras. Accordingly, the one or more cameras 48 may consist of a single camera or may comprise more than two cameras. Head-mounted display unit 28 may be provided in the form of eyewear, such as glasses as shown in FIG. 2A or goggles. Alternatively, head-mounted display unit 28 may be provided in the form of an over-the-head or forehead-mounted headset 70, as shown in FIG. 2B.

FIG. 41 is a flow chart that schematically illustrates a method for generation and display of a 3D model including bone and soft tissue information based on registering preoperative 3D MR images and intraoperative 2D fluoroscopic images, in accordance with an embodiment of the disclosure. Although the method is described here in connection with vertebrae of the spine, the method may be similarly used for other bones, such as shoulder bones, hip bones, knee bones, leg bones, arm bones, foot bones, ankle bones, bones of the head, etc. Further details of this method are described herein.

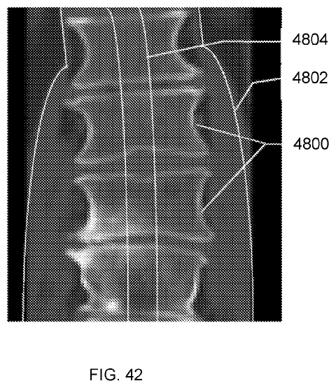

To begin the process of registering the preoperative 3D MR image segments with the 2D images of the spine, processor 50 segments an MR image of the patient's back into bone segments and soft tissue in proximity to the bone segments (Block 4700), for example as shown in FIG. 42. At Block 4702, processor 50 receives an initial input associating one of the vertebrae among the 3D image segments with the locations of the same vertebra in the two 2D images. For example, a user of system 20 may use a cursor to mark the location of a selected 3D vertebra on the 2D images. In addition, in some embodiments, processor 50 makes an initial estimate of the orientation of the spine of patient 24 using external cues. For example, the location of marker 44 or 46 relative to the patient's skeleton indicates the Z-direction (e.g., the sagittal axis), while locations of X-ray source 32 and detector 34 indicate the Y-direction (e.g., the longitudinal axis). Alternatively, the user changes the orientation of the 2D images to match the orientation of the 3D image segments. Based on the initial input and the estimated orientation of the spine, processor 50 is able to associate each of the 3D image segments with a corresponding vertebra in each of the 2D images (Block 4704). Processor 50 may also estimate and make use of the known ranges of movement of the vertebrae relative to one another in estimating the registration parameters.

To register the vertebrae in the 3D MR image segments precisely with the associated vertebrae in the 2D images, processor 50 generates (e.g., calculates) digitally reconstructed radiographs (DRRs) based on the 3D images of the vertebrae over a range of vertebral movements and rotations around the estimated axes of the 2D images relative to the spine (Block 4706). In some embodiments, the intensity of each pixel in a given DRR is computed by calculating the cumulative radiodensity of the voxels along the path of a ray between the X-ray source and the pixel. In one example, processor 50 applies a process of optimization to find the orientation of each 3D vertebra relative to the 2D images by comparing the gradients of the pixel values in the DRR to the actual gradients of the pixel values in the 2D X-ray images.

Once the optimal locations and orientations of all the vertebrae in the 3D MR image segments have been found in this manner, processor 50 uses the results in reconstructing a complete 3D model of the spine from the individual 3D vertebrae (Block 4708). In accordance with several embodiments, the locations and orientations of the vertebrae in this 3D model will match the actual spine (e.g., the actual pose of the spine) of patient 24 on operating table 26. The locations and orientations of the soft tissues in the segmented MR image in proximity to the vertebrae may be reconstructed using the same transformation parameters as were generated in reconstructing the 3D model of the spine, so that the entire patient anatomy is properly rendered and registered with the underlying tissues.

Processor 50 may then display the 3D model (or generate the 3D model as output for display), including both bones and soft tissues at Block 4710 (e.g., to facilitate navigation of medical tools in the procedure). Image registration and fusion modes that may be used for this purpose are shown by way of example in FIGS. 43A, 43B, and 43C. When AR is utilized, processor 50 may then use the relative location and orientation of head-mounted AR display unit 28 or head-mounted AR display unit 70 with respect to patient 24 to calculate the views of the vertebrae and soft tissues that will be projected onto displays 60, 72 in the proper locations and orientations, overlaid on the actual anatomy of patient 24.

FIG. 42 is a schematic representation of a segmented 3D image for display in image-guided surgery, in accordance with an embodiment of the disclosure. This image is generated by processing and segmentation of an MR image of a patient and includes vertebrae 4800 surrounded by muscle tissue 4802. The image may also be enhanced (e.g., with virtual graphics) to show, for example, the location of a spinal cord 4804 passing through and between vertebrae 4800, as well as peripheral nerves (not shown) branching from the spinal cord or tumors (also not shown).

The image shown in FIG. 42 may be presented, for example, on displays 60 (FIG. 2A) or displays 72 (FIG. 2B), in registration with the underlying anatomical structures. Alternatively or additionally, this image may be presented on a separate display (local and/or remote).

### Fusion of Multiple Image Modalities

FIGS. 43A-43C are flow charts that schematically illustrate modalities for image registration, fusion, and/or display, using the tools and techniques described above, in accordance with embodiments of the disclosure.

In the example of FIG. 43A, a preoperative MR image is captured (Block 4900) and converted by processor 50 or by another processor or processors to a 3D image showing bone structure, simulating and/or imitating a CT image, and indicated in FIGS. 43A-43C as “Bone MR image (Block 4902)” The Bone MR image may be used as a substitute or instead of a CT image. The MR image may be converted to Bone MR image by various techniques. Such techniques may include, for example, using BoneMRI™ Software, deep neural networks (e.g., U-Net or DenseNet), and/or other image processing techniques such as active contours or level-sets. Such conversion techniques may include enhancing the bone tissue and/or segmenting the bone tissue to distinguish bone from surrounding soft tissue. Intraoperative 2D X-ray images are captured, for example using fluoroscope 30 (FIG. 1). At Block 4906), the processor registers the bone segments or portions in the Bone MR image with the corresponding bone segments or portions in the one or more 2D X-ray images, as described above, and the resulting registered image data displaying up-to-date bone tissue data are presented on a display, for example, but not limited to, an augmented-reality (AR) display (Block 4908).

The embodiment of FIG. 43B uses a similar process to generate an up-to-date Bone MR image, by registering it with a preoperative 2D Xray image (Block 4904) and as described with respect to FIG. 43A. Processor 50 or another processor generates a registered fused MR image (including both bones and soft tissue) at Block 4906 by utilizing the registered Bone MR image for up-to-date bone tissue data and the original MR image for soft tissue data, while the Bone MR image and the original MR image are inherently registered one with the other. The processor may then present the fused image on a display, optionally, an AR display (Block 4908).

In the example of FIG. 43C, registered CT-MR fused image data are displayed. A CT image is used for providing bone tissue data while an MR image is used for providing soft tissue data. The CT bone tissue data and the MR soft tissue data are fused to generate a fused image. An MR image is typically generated preoperatively. A CT image may be generated intraoperatively (Block 4914) or preoperatively (Block 4912). When the CT image is generated preoperatively as well, an intraoperative 2D Xray image may be captured and registered with the CT preoperative image to provide up-to-date bone tissue data (Block 4916), for example as described herein above with respect to FIG. 41. The intraoperative CT image or the registered preoperative CT image may be then fused with the MR image (Block 4918). In some embodiments, a Bone MR image may be generated, for example as described with respect to FIG. 43A, to facilitate the fusion of the MR image with the CT image. In some embodiments, the CT image (preoperative or intraoperative) may be segmented to define the bone tissue and to facilitate the CT image fusion with the MR image. Processor 50 or another processor or processors may be utilized to perform the steps described hereinabove. In some embodiments, the processor(s) may register the preoperative MR image with the segmented CT image, so that the soft tissues in the MR image are properly aligned with the bones in the CT image. (If the MR image has been segmented to identify bone segments, these bone segments may be used to facilitate accurate registration of the MR image with the CT image.) Once the MR and CT images have been registered with one another, the processor fuses the data from the images and presents the resulting fused image, including both bones and soft tissue, on a display, optionally, an AR display. Persons skilled in the art may implement different methods for registering and fusing the CT image data with the MR image data.

The display of the fused image may include, for example, different colors for the different tissues, color for one type of tissue and black-white for another type of tissue, or the tissue colors may be gray scale corresponding to pixel intensity. Alternatively, or additionally, toggling between display modes displaying different types of tissue (for example bone tissue image vs. soft tissue image), while the images are registered and optionally displayed in alignment, may be provided.

The display of such a fused image may be advantageous in various types of medical procedures. For example, in bone-related procedures, soft tissue information may provide information with respect to critical structures, such as nerves in spine procedures. As another example, in soft-tissue-related procedures such as removal of tumors, bone information may facilitate access and navigation.

In some embodiments, the fused image may be presented as a 3D image, for example via a 3D model. In some embodiments, 2D images including 2D slices of the fused image may be displayed. The 3D and/or 2D images may be presented in various views, including axial, sagittal, lateral and/or anteroposterior (AP) views. The display may provide necessary information during a medical procedure and/or facilitate navigation. In head-mounted AR systems, such as the head-mounted displays shown in FIGS. 2A and 2B, the fused image may be displayed from the point of view of a professional wearing the head-up display. In some embodiments, the fused image may be displayed from a point of view of a tip of a medical tool inserted into and navigated within the patient body.

In some embodiments, a fused image generated only based on pre-operative MRI utilizing the generation of a Bone MR image or generated based on a registration between preoperative MR and CT images, as disclosed herein, may be used in a planning phase of a medical procedure or intervention.

### Distortion Correction

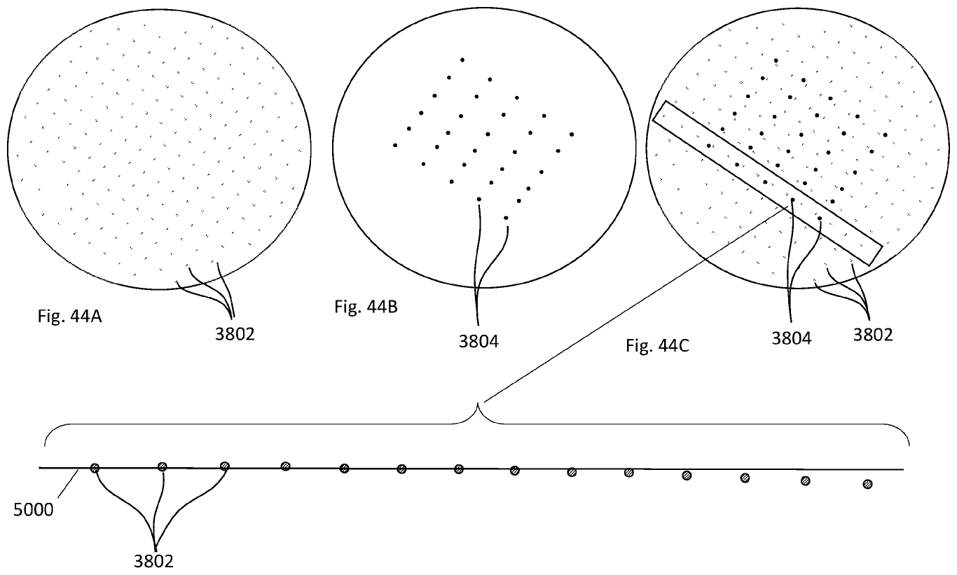

X-ray images possess distortion that most closely resembles a combination of s-distortion and pincushion distortion. This distortion-type is approximately illustrated through the image of the bead plates in FIGS. 44A-44C. FIG. 44C further demonstrates the presence of the distortion in a magnified view of one row of the image of beads 3802 fitted to an undistorted line 5000. Distortion correction algorithms and processes that normally work on regular camera images do not work to correct for X-ray image distortion. In some implementations, a two-step approach is utilized to correct the distortion in the X-ray images: (1) image data refinement and (2) spline interpolation.

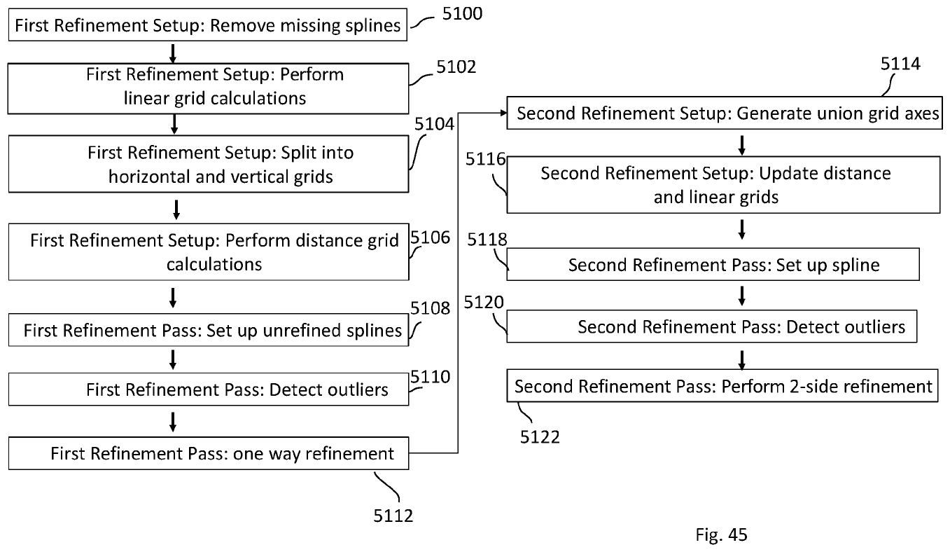

FIG. 45 is a flowchart that schematically illustrates an example method for refining image data as part of a distortion correction process or algorithm, which may be executed by one or more processors (e.g., processor 50). In some instances, the image data after a bead detection algorithm has been run can comprise outliers and/or missing beads, which can produce artifacts. In some implementations, a refinement algorithm is used to improve the image data. In one implementation, the refinement algorithm includes a first refinement setup, a first refinement pass, a second refinement setup, and a second refinement pass.

In some implementations, the first refinement setup can include steps 5100, 5102, 5104, and 5106. In some implementations, a bead detection algorithm, such as the one described herein, has been performed on the X-ray image of the beads (e.g., beads of the upper bead plate 406) and the resulting detected beads make up a grid of source points (e.g., a grid of observed bead points or control points (for the purposes of spline calculations or interpolation)). A grid of target points (e.g., a grid of ideal or expected bead points) is created using prior knowledge comprising information like the bead quantity, sizing, and spacing. Utilizing the grid of source points (e.g., source grid) and the grid of target points (e.g., target grid), at step 5100, missing splines are removed. For example, the two grids are compared and where the source grid is missing a row or column of source points, the corresponding target points in the target grid are removed. As previously mentioned, not all beads may be detected with the bead detection algorithm. At step 5102, straight lines can be calculated from the existing source or control points and where there appear to be gaps indicative of missing source points, new source points (e.g., generated source points) can be added to fill the source grid. At step 5104, the grids may be split into horizontal and vertical grids and the source point lines are filtered out if the total number of source points within an individual source point line falls below a threshold value. These lines are not included in subsequent spline calculations. In some implementations, splines can be built using two source points. In some implementations, the splines can be built using more than two source points. At step 5106, distance grids are calculated where grid points are given scores based on how far away they are from the original source points. For example, a generated source point located one space away from an original source point can be provided a distance value of one, and a generated source point located two spaces away from an original source point can be provided a distance value of two.

In some implementations, the first refinement pass can include steps 5108, 5110, and 5112. The first refinement pass may take place upon completion of the first refinement setup sub-process. At step 5108, unrefined splines are set up and using the splines that have been computed for the source grid, the intersections of the computed splines with the target lines are set as synthetic source grid points. At step 5110, outlier source points are detected and marked based at least on prior knowledge of the beads and bead pattern. At step 5112, one-way refinement may be performed. This refinement step uses the data of neighboring points to refine the source points. For example, in one embodiment the computed distance grid map determines which neighbor points are closest to each of the original source points, derivatives of the neighbor spline are computed, and for each control point, the algorithm determines which neighbor spline has a better score and matches the derivative accordingly. The result is a source grid comprising refined source or control points.

In some implementations, the second refinement setup can include steps 5114 and 5116. At step 5114, union grid axes are generated. The refined control points from the first refinement pass are used to determine all the vertical and horizontal splines. A new source grid is created, such that the new source grid contains the source points comprising the points at the intersections between splines and for target lines with missing splines. At step 5116, the distance and linear grids are updated as was done at steps 5106 and 5108 but using the new synthetic source grid.

In some embodiments, the second refinement pass can include steps 5118, 5120, and 5122. Step 5118 may be carried out at the conclusion of the second refinement setup sub-process. At step 5118, splines are calculated for the new source grid. At step 5120, outliers are detected, and at step 5122, the refinement step is performed. This refinement step is done by calculating a weighted average of the source point and its neighboring points (e.g., neighboring points not marked as outliers and possessing low distance grid values). Following this refinement step, the result is a refined source grid and a target grid to be used in a distortion correction algorithm.

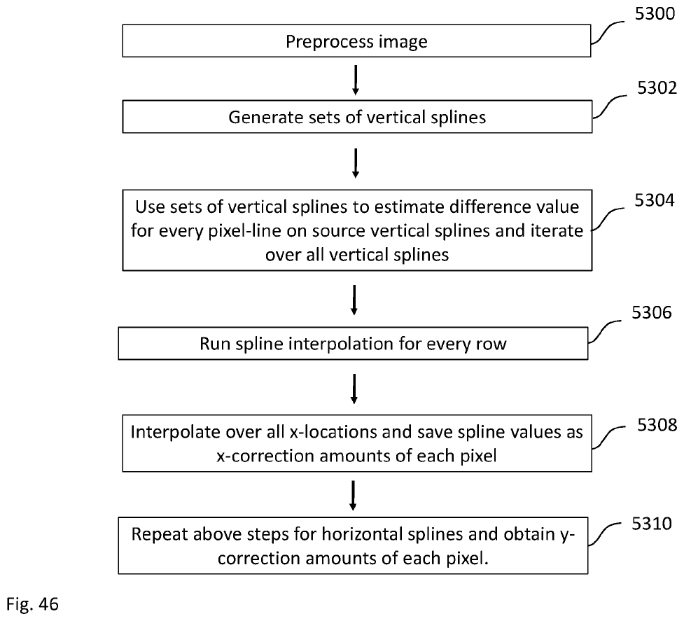

In FIG. 46, a flowchart schematically illustrates an example method for interpolating data as part of a distortion correction process in accordance with embodiments of the disclosure. At step 5300, the X-ray image is pre-processed. According to one embodiment, a schematic of an X-ray image of a superposition of the beads (e.g., beads of the upper bead plate 406 and lower bead plate 408) is acquired. The image of the source beads (e.g., source grid) may be rotated and aligned with (X,Y) axes, and the grid of target beads may be generated and aligned with the center location of the source grid. At step 5302, vertical splines are generated for the source and target grids. In some implementations, splines for the source points are computed using source points residing on the same columns of the source grid and splines for the target points are computed using the y-positions of the source grid points and the x-positions of the target grid points. At step 5304, the differences between the vertical spline generated from the source points and the vertical spline generated from the target and source points is estimated for each source point and repeated over all vertical splines. The differences are plotted as a function of the source point y-axis value, and fitted to a spline. Using this fitted spline, the difference value for every pixel-line on the source spline may be estimated. This process may be repeated over all vertical splines. This spline interpolation process may then be repeated for every row of the source image at step 5306, and the determined differences may be plotted as a function of the source point x-axis value and fitted to a spline, from which the difference value for every pixel-line on the source spline is estimated (e.g., interpolation occurs over all x-locations of the source image). The spline value may be saved or stored in memory as the x-correction amount (step 5308). At step 5310 of the distortion correction algorithm, steps 5302 through 5308 are repeated for the horizontal splines to obtain the y-correction amounts for each pixel. The interpolation process yields x-axis and y-axis corrections for every pixel in the source image, and an undistorted image can be created by resampling the target pixels from the source image values.

### Conclusion and Terminology

While examples of the disclosed technique are given for body portion containing spine vertebrae, the principles of the system, method, and/or disclosure may also be applied to other bones and/or body portions than spine, including hip bones, pelvic bones, leg bones, arm bones, ankle bones, foot bones, shoulder bones, cranial bones, oral and maxillofacial bones, sacroiliac joints, etc.

The disclosed technique is presented with relation to image-guided surgery systems or methods, in general, and accordingly, the disclosed technique of visualization of medical images should not be considered limited only to augmented reality systems and/or head-mounted systems. For example, the technique is applicable to the processing of images from different imaging modalities, as described above, for use in diagnostics.

The terms “top,” “bottom,” “first,” “second,” “upper,” “lower,” “height,” “width,” “length,” “end,” “side,” “horizontal,” “vertical,” and similar terms may be used herein; it should be understood that these terms have reference only to the structures shown in the figures and are utilized only to facilitate describing embodiments of the disclosure. Various embodiments of the disclosure have been presented in a range format. It should be understood that the description in range format is merely for convenience and brevity and should not be construed as an inflexible limitation on the scope of the disclosure. The ranges disclosed herein encompass any and all overlap, sub-ranges, and combinations thereof, as well as individual numerical values within that range. For example, description of a range such as from about 5 to about 30 degrees should be considered to have specifically disclosed subranges such as from 5 to 10 degrees, from 10 to 20 degrees, from 5 to 25 degrees, from 15 to 30 degrees etc., as well as individual numbers within that range (for example, 5, 10, 15, 20, 25, 12, 15.5 and any whole and partial increments therebetween). Language such as “up to,” “at least,” “greater than,” “less than,” “between,” and the like includes the number recited. Numbers preceded by a term such as “about” or “approximately” include the recited numbers. For example, “approximately 2 mm” includes “2 mm.” The terms “approximately”, “about”, and “substantially” as used herein represent an amount close to the stated amount that still performs a desired function or achieves a desired result.

In some embodiments, the system comprises various features that are present as single features (as opposed to multiple features). For example, in one embodiment, the system includes a single HMD, a single camera, a single processor, a single display, a single marker, a single calibration jig, a single image, a single bead plate, a single imaging device, a single fluoroscope, etc. Multiple features or components are provided in alternate embodiments.

In some embodiments, the system comprises one or more of the following: means for imaging (e.g., a camera or fluoroscope or MRI machine or CT machine), means for calibration (e.g., calibration jigs), means for registration (e.g., adapters, markers, objects, cameras), means for fastening (e.g., anchors, adhesives, clamps, pins), means for segmentation (e.g., one or more neural networks), means for distortion correction (e.g., ring markers and grids of beads), etc.

The processors described herein may include one or more central processing units (CPUs) or processors or microprocessors. The processors may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors (or memory units communicatively coupled thereto) may include modules comprising program instructions or algorithm steps configured for execution by the processors to perform any of all of the processes or algorithms discussed herein. The processors may be communicatively coupled to external devices (e.g., display devices, data storage devices, databases, servers, etc. over a network via a network communications interface.

In general, the algorithms or processes described herein can be implemented by logic embodied in hardware or firmware, or by a collection of software instructions, possibly having entry and exit points, written in a programming language, such as, for example, Python, Java, Lua, C, C#, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the computing system 50, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware. Generally, any modules or programs or flowcharts described herein may refer to logical modules that may be combined with other modules or divided into sub-modules despite their physical organization or storage.

The various features and processes described above may be used independently of one another or may be combined in various ways. All possible combinations and sub-combinations are intended to fall within the scope of this disclosure. In addition, certain method or process blocks or steps may be omitted in some implementations. The methods and processes described herein are also not limited to any particular sequence, and the blocks, steps, or states relating thereto can be performed in other sequences that are appropriate. For example, described blocks, steps, or states may be performed in an order other than that specifically disclosed, or multiple blocks or states may be combined in a single block or state. The example blocks, steps, or states may be performed in serial, in parallel, or in some other manner. Blocks, steps, or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process.

It will be appreciated that the systems and methods of the disclosure each have several innovative aspects, no single one of which is solely responsible or required for the desirable attributes disclosed herein. The various features and processes described above may be used independently of one another or may be combined in various ways. The section headings used herein are merely provided to enhance readability and are not intended to limit the scope of the embodiments disclosed in a particular section to the features or elements disclosed in that section.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

1. A method for facilitating augmented-reality assisted navigation based on pre-operative 3D imaging and intraoperative 2D imaging of at least a portion of a spine of a patient, the method comprising:
receiving a three-dimensional (3D) tomographic image of at least the portion of the spine of the patient, the portion of the spine comprising multiple vertebrae and/or other bony structures;
segmenting the 3D tomographic image into a plurality of 3D segments, each comprising a respective vertebra of the multiple vertebrae;
receiving two or more two-dimensional (2D) fluoroscopic images of at least the portion of the spine of the patient;
registering each respective 3D segment of the plurality of 3D segments with a respective vertebra of the multiple vertebrae in the two or more 2D fluoroscopic images;
generating a 3D image volume of at least the portion of the spine based on the registering; and
presenting the 3D image volume of at least the portion of the spine on an augmented-reality display.

2. The method of claim 1, wherein the 3D tomographic image is a computed tomography (CT) image.

3. The method of claim 1, wherein segmenting the 3D tomographic image into a plurality of 3D segments is performed automatically by one or more processors.

4. The method of claim 1, wherein segmenting the 3D tomographic image into a plurality of 3D segments is performed via application of one or more convolutional neural networks.

5. The method of claim 1, wherein segmenting the 3D tomographic image into a plurality of 3D segments comprises labeling of the multiple vertebrae.

6. The method of claim 1, wherein receiving the two or more 2D fluoroscopic images comprises receiving a first 2D fluoroscopic image captured from a first angle or viewpoint of a C-arm fluoroscope and receiving a second 2D fluoroscopic image captured from a second angle or viewpoint of the C-arm fluoroscope different from the first angle or viewpoint.

7. The method of claim 1, wherein presenting the 3D image volume comprises overlaying an augmented reality image of the registered plurality of 3D segments on a back of the patient.

8. The method of claim 7, further comprising calibrating a frame of reference of the 2D fluoroscopic images relative to the spine of the patient prior to the registering, wherein overlaying the augmented reality image comprises applying the calibrated frame of reference in overlaying the vertebrae in the registered plurality of 3D segments on the spine of the patient.

9. The method of claim 1, wherein receiving the two or more 2D fluoroscopic images comprises receiving two 2D fluoroscopic images captured from different, respective angles relative to the patient.

10. The method of claim 9, wherein registering each respective 3D segment of the plurality of 3D segments comprises aligning each of the plurality of 3D segments with both of the two 2D fluoroscopic images.

11. The method of claim 8, wherein calibrating the frame of reference of the 2D fluoroscopic images comprises performing distortion correction of the two or more 2D fluoroscopic images.

12. The method of claim 11, wherein performing distortion correction comprises spline interpolation.

13. The method of claim 1, wherein registering each respective 3D segment of the plurality of 3D segments comprises adjusting a respective location and orientation of each respective 3D segment of the plurality of 3D segments to match the respective vertebra of the multiple vertebrae in the two or more 2D fluoroscopic images.

14. The method of claim 13, wherein,
adjusting the respective location and orientation comprises processing each respective 3D segment of the plurality of 3D segments to generate digitally reconstructed radiographs (DRRs), and
the respective location and orientation are adjusted to maximize a similarity between the DRRs and the two or more 2D fluoroscopic images.

15. The method of claim 1, wherein the augmented-reality display is a see-through stereoscopic display of a wearable device.

16. The method of claim 15, wherein the wearable device comprises glasses.

17. The method of claim 15, wherein the wearable device is a head-mounted unit.

18. The method of claim 1, wherein registering each 3D segment of the plurality of 3D segments comprises performing initial guess estimation.

19. The method of claim 1, wherein the 3D image volume comprises a reconstructed 3D model of the multiple vertebrae.

20. The method of claim 1, wherein:
the 3D tomographic image is a computed tomography (CT) image;
the augmented-reality display is a see-through stereoscopic display of a wearable device;
receiving the two or more 2D fluoroscopic images comprises receiving a first 2D fluoroscopic image captured from a first angle or viewpoint of a C-arm fluoroscope and receiving a second 2D fluoroscopic image captured from a second angle or viewpoint of the C-arm fluoroscope different from the first angle or viewpoint;
presenting the 3D image volume comprises overlaying an augmented reality image of the registered plurality of 3D segments on a back of the patient;
registering each respective 3D segment of the plurality of 3D segments comprises adjusting a respective location and orientation of each respective 3D segment of the plurality of 3D segments to match the respective vertebra in the two or more 2D fluoroscopic images; and
adjusting the respective location and orientation comprises processing each 3D segment to generate digitally reconstructed radiographs (DRRs), and finding an optimal match between the DRRs and the respective vertebra in the one or more 2D fluoroscopic images.

21-104. (canceled)
